(19) United States
(12) Patent Application Publication
(10) Pub. No.: US 2026/0293515 A1
(43) Pub. Date: Sep. 24, 2026

(54) HOST MATERIAL FOR ORGANIC ELECTROLUMINESCENT ELEMENTS, PRELIMINARY MIXTURE, AND ORGANIC ELECTROLUMINESCENT ELEMENT

(71) Applicant: NIPPON STEEL Chemical & Material Co., Ltd., Tokyo (JP)

(72) Inventors: Junya OGAWA, Tokyo (JP); Mitsuru SAKAI, Tokyo (JP); Koyo TAKAKI, Tokyo (JP); Yuji IKENAGA, Tokyo (JP)

(73) Assignee: NIPPON STEEL Chemical & Material Co., Ltd., Tokyo (JP)

(21) Appl. No.: 19/100,620

(22) PCT Filed: Aug. 28, 2023
(86) PCT No.: PCT/JP2023/031041

§ 371 Date: Feb. 3, 2025

(30) Foreign Application Priority Data
Aug. 31, 2022 (JP) 2022-138388

(51) Int. Cl. H10K 85/60; C07B 59/00; C07D 487/04; C09K 11/02; C09K 11/06; H10K 50/11; H10K 85/30; H10K 101/00

(52) U.S. Cl. CPC H10K 85/654; C07B 59/002; C07D 487/04; C09K 11/02; C09K 11/06; H10K 85/342; H10K 85/6572; C09K 2211/1007; C09K 2211/1029; C09K 2211/185; H10K 50/11; H10K 2101/90

(57) ABSTRACT

An object is to provide a practically useful organic EL device having a low voltage, high efficiency, and extended lifetime characteristics. A host material for an organic electroluminescent device represented by the following general formula (1) is used:

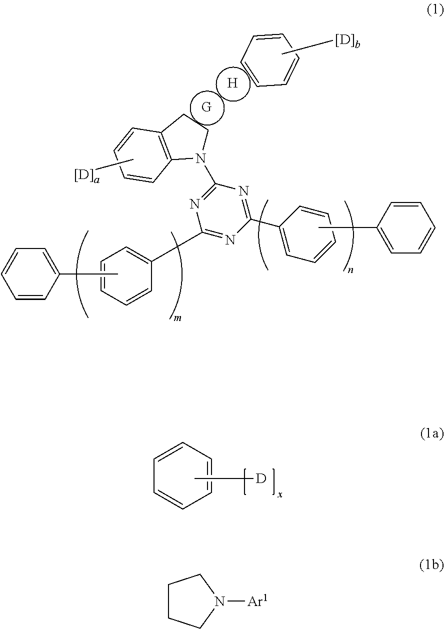

wherein, ring G represents an aromatic ring represented by a formula (1a), ring H represents a heterocyclic ring represented by a formula (1b), D represents deuterium, and Ar¹ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five aromatic rings of these aromatic groups are linked to each other, a, b, and x represent the number of substitutions, a and b represent an integer of 0 to 4, x represents an integer of 0 to 2, and a+b+x≥1, m and n represent the number of repetitions, m and n represent an integer of 0 or 1, and m+n is 1 or more.

### TECHNICAL FIELD

The present invention relates to an organic electroluminescent element or device (hereinafter, referred to as an organic EL device), and specifically relates to an organic EL device comprising a specific mixed host material.

### BACKGROUND ART

Application of a voltage to an organic EL device allows injection of holes and electrons from an anode and a cathode, respectively, into a light-emitting layer. Then, in the light-emitting layer, injected holes and electrons recombine to generate excitons. At this time, according to statistical rules of electron spins, singlet excitons and triplet excitons are generated at a ratio of 1:3. Regarding a fluorescence-emitting organic EL device using light emission from singlet excitons, it is said that the internal quantum efficiency thereof has a limit of 25%. Meanwhile, regarding a phosphorescent organic EL device using light emission from triplet excitons, it is known that intersystem crossing is efficiently performed from singlet excitons, the internal quantum efficiency is enhanced to 100%.

Highly efficient organic EL devices utilizing delayed fluorescence have been developed recently. For example, Patent Literature 1 discloses an organic EL device utilizing a TTF (Triplet-Triplet Fusion) mechanism, which is one of delayed fluorescence mechanisms. The TTF mechanism utilizes a phenomenon in which singlet excitons are generated due to collision of two triplet excitons, and it is thought that the internal quantum efficiency can be theoretically raised to 40%. However, since the efficiency is lower compared to phosphorescent organic EL devices, further improvement in efficiency and low voltage characteristics are required.

In addition, patent Literature 2 discloses an organic EL device utilizing a TADF (Thermally Activated Delayed Fluorescence) mechanism. The TADF mechanism utilizes a phenomenon in which reverse intersystem crossing from triplet excitons to singlet excitons is generated in a material having a small energy difference between a singlet level and a triplet level, and it is thought that the internal quantum efficiency can be theoretically raised to 100%.

However, all the mechanisms have room for advancement in terms of both efficiency and lifetime, and are additionally required to be improved also in terms of reduction in driving voltage.

On the other hand, Patent Literature 3 discloses use of an indolocarbazole compound as a host material of a light emitting layer.

Patent Literatures 4 and 5 disclose use of an indolocarbazole compound and a biscarbazole compound in a mixed host material of a light-emitting layer.

Patent Literatures 6 and 7 disclose use of a deuterium-substituted indolocarbazole compound in a host material of a light-emitting layer.

Patent Literatures 8 and 9 disclose use of a deuterated biscarbazole compound in a host material of a light-emitting layer.

Patent Literatures 7 and 10 disclose use of a deuterium-substituted indolocarbazole compound and a biscarbazole compound in a mixed host material of a light emitting layer.

However, none of these can be said to be sufficient, and further improvement is desired.

### CITATION LIST

### Patent Literature

Patent Literature 1: WO2010/134350 A
Patent Literature 2: WO2011/070963 A
Patent Literature 3: WO2008/056746 A
Patent Literature 4: WO2018/198844 A
Patent Literature 5: U.S. Ser. No. 10/333,077 A
Patent Literature 6: JP5784621 B1
Patent Literature 7: KR102054806 B1
Patent Literature 8: KR102283849 B1
Patent Literature 9: KR2022-0013910 A
Patent Literature 10: KR102193015 B1

### SUMMARY OF INVENTION

### Technical Problem

Organic EL displays, when compared with liquid crystal displays, are not only characterized by being thin-and-light, high in contrast, and capable of displaying a high-speed moving picture, but also highly valued in terms of designability such as curving and flexibility, and are widely applied in display apparatuses including mobiles and TV. However, organic EL displays are needed to be further reduced in voltage in order to suppress battery consumption in the case of use thereof for mobile terminals, and are inferior as light sources in terms of luminance and lifetime as compared with inorganic LEDs and thus are demanded to be improved in efficiency and enhanced in device lifetime. In view of the above circumstances, an object of the present invention is to provide a practically useful organic EL device having a low voltage, high efficiency and lifetime characteristics.

As a result of intensive studies, the present inventors have found that the above problems can be solved by an organic electroluminescent device using a specific host material, in particular, a mixed host material containing the specific host material in a light emitting layer, and have completed the present invention.

The present invention relates to a host material for an organic electroluminescent device represented by the following general formula (1):

### [C1]

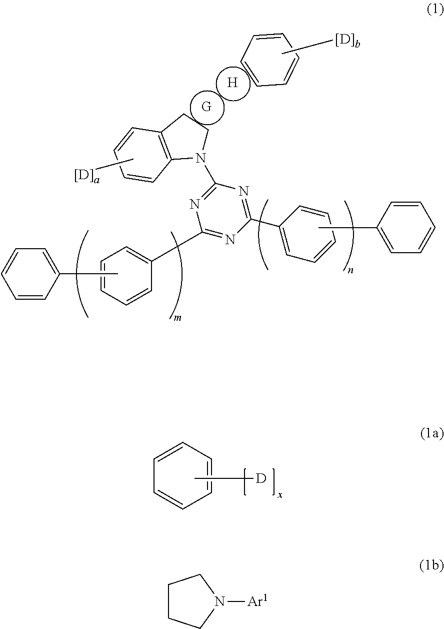

In the general formula (1), ring G represents an aromatic ring represented by a formula (1a), and ring H represents a heterocyclic ring represented by a formula (1b).

D represents deuterium, Ar¹ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five aromatic rings of these aromatic groups are linked to each other. a, b, and x represent the number of substitutions, a and b independently represent an integer of 0 to 4, x represents an integer of 0 to 2, and a+b+x≥1. m and n represent the number of repetitions, m and n each independently represent an integer of 0 or 1, and m+n is 1 or more.

In the general formula (1), the substituent substituted with triazine and Ar¹ contain no deuterium.

In a preferred aspect of the present invention, any of the following is satisfied: Ar¹ is a substituted or unsubstituted aromatic hydrocarbon group having 6 to 18 carbon atoms or a substituted or unsubstituted linked aromatic group in which two aromatic rings of such an aromatic group are linked to each other, Ar¹ is a substituted or unsubstituted phenyl group or a substituted or unsubstituted biphenyl group, and m represents 0, in the general formula (1).

A preferred aspect of the general formula (1) is any of the following (2) to (5):

### [C2]

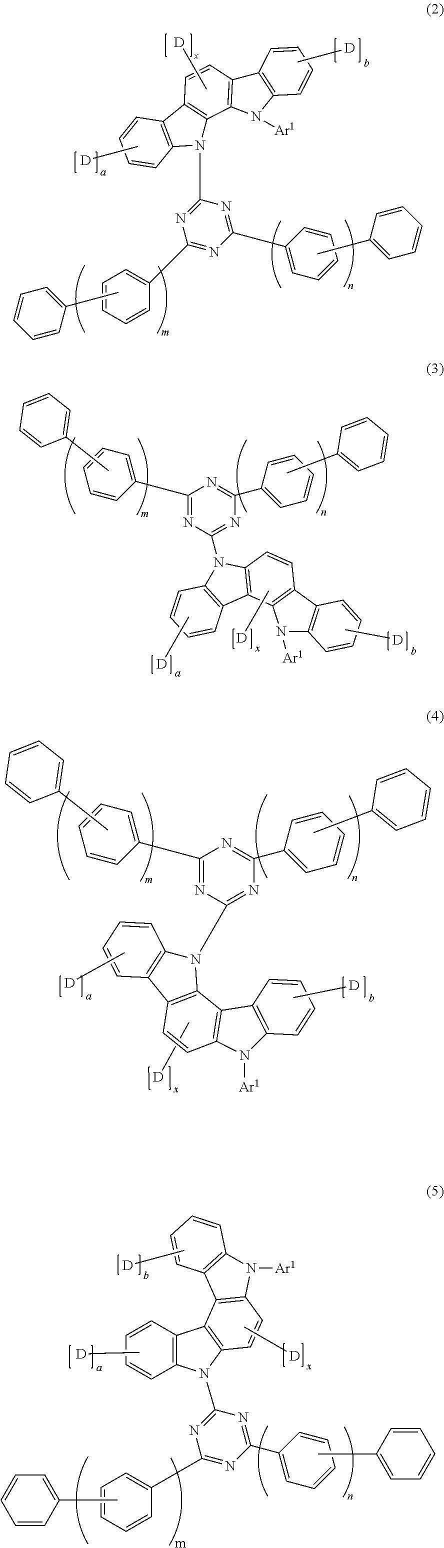

The present invention relates to an organic electroluminescent device comprising one or more light emitting layers between an anode and a cathode opposed to each other, wherein at least one of the light emitting layers contains a first host selected from the compound represented by the general formula (1), a second host selected from the compound represented by the following general formula (6), and a light emitting dopant material.

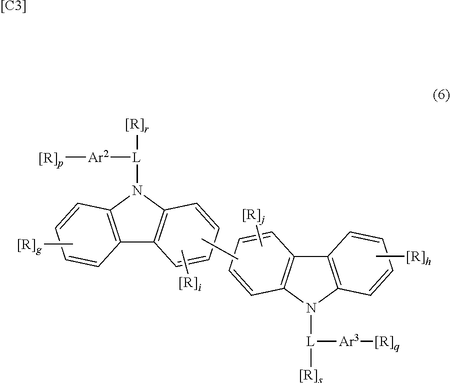

In the formula (6), Ar² and Ar³ each independently represent a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five aromatic rings of these aromatic groups are linked to each other.

L each independently represents a single bond, a substituted or unsubstituted aromatic hydrocarbon group having 6 to 18 carbon atoms, or a substituted or unsubstituted aromatic heterocyclic group having 3 to 17 carbon atoms, and each R independently represents deuterium or an aliphatic hydrocarbon group having 1 to 10 carbon atoms.

g to j and p to s represent the number of substitutions, g, h, r, and s each independently represent an integer of 0 to 4, i and j each independently represent an integer of 0 to 3, and p and q each independently represent an integer of 0 to 13. Here, when L is a single bond, r and s represent an integer of 0.

In an aspect, the compound of the general formula (6) is represented by the following formula (7).

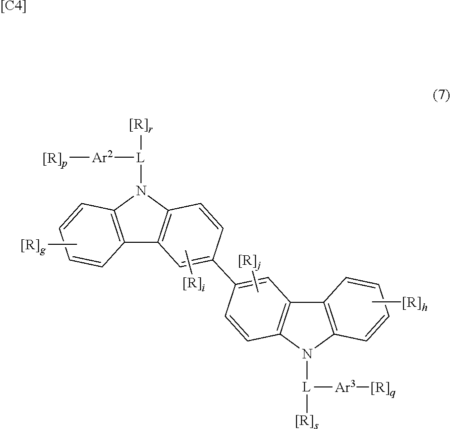

In the general formula (6), Ar² and Ar³ preferably each independently represent a substituted or unsubstituted phenyl group, a substituted or unsubstituted biphenyl group, or a substituted or unsubstituted terphenyl group, and the terphenyl group may be linked linearly or branched. A host material in which R is deuterium is preferred.

The organic electroluminescent device of the present invention comprises a light-emitting layer having a mixed host containing two compounds and having a dopant (light-emitting dopant material). In the organic electroluminescent device, the ratio of the compound represented by the general formula (1) to the total of the compound represented by the general formula (1) and the compound represented by the general formula (6) in the mixed host is preferably 10 mass % or more and less than 80 mass %, and more preferably 20 mass % or more and less than 70 mass %. It is more preferable that the light-emitting dopant be an organic metal complex containing at least one metal selected from the group consisting of ruthenium, rhodium, palladium, silver, rhenium, osmium, iridium, platinum and gold, or a thermally activated delayed fluorescence-emitting dopant.

The present invention relates to a premixture used for forming a light-emitting layer for an organic electroluminescent device comprising a light-emitting layer containing hosts and a light-emitting dopant material between an anode and a cathode opposed to each other, wherein the premixture contains a first host and a second host, and among these, the first host is selected from a compound represented by the general formula (1), and the second host is selected from a compound represented by the general formula (6).

Production of the organic electroluminescent device preferably comprises a step of mixing the first host represented by the general formula (1) and the second host represented by the general formula (6) to give a premixture and then vapor-depositing a host material containing the premixture to form a light-emitting layer.

In a method for producing the organic electroluminescent device, a difference in temperature at 50% weight loss of the first host and the second host is preferably within 20° C.

### Advantageous Effect of Invention

According to the present invention, a compound represented by the general formula (1) has indolocarbazole and a nitrogen-containing six-membered ring, the indolocarbazole is substituted with deuterium, and the nitrogen-containing six-membered ring bonded to the indolocarbazole has one or more phenylene groups. Then, the compound represented by the general formula (1) can be used as a host material, suitably, the compound represented by the general formula (1) as a first host and a biscarbazole compound represented by the general formula (6) as a second host can be mixed and used to thereby obtain an organic EL device having high efficiency and extended lifetime in spite of having a low voltage.

### BRIEF DESCRIPTION OF DRAWING

FIG. 1 is a schematic cross-sectional view showing one example of an organic EL device.

### DESCRIPTION OF EMBODIMENTS

The host material for an organic EL device of the present invention is represented by the general formula (1).

In the general formula (1), ring G represents an aromatic ring represented by the formula (1a), and is fused with two adjacent rings. Ring H represents a five-membered heterocyclic ring represented by the formula (1b), and is fused with two adjacent rings at any positions, but is not fused on a side containing N. Accordingly, the indolocarbazole ring has some isomeric structures, though the number is limited. The compound represented by the general formula (1) can be specifically represented as any of the structures represented by the formulas (2) to (5), and in a preferred aspect, the compound is represented as any of the structures represented by the formulas (2) to (4), and in a more preferred aspect represented as the structure represented by the formula (2).

In the general formula (1) and the formulas (2) to (5), the same symbols have the same meaning. D represents deuterium, and Ar¹ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five aromatic rings of these aromatic groups are linked to each other. Preferred is a substituted or unsubstituted aromatic hydrocarbon group having 6 to 18 carbon atoms or a substituted or unsubstituted linked aromatic group in which two aromatic rings of these aromatic groups are linked to each other, and more preferred is a substituted or unsubstituted phenyl group or a substituted or unsubstituted biphenyl group.

m and n represent the number of repetitions, m and n each independently represent an integer of 0 or 1, and m+n is 1 or more. It is preferable that m represent an integer of 0 and n represent an integer of 1.

a, b, and x represent the number of substitutions, a and b each independently represent an integer of 0 to 4, x represents an integer of 0 to 2, and a+b+x≥1, and it is preferable that a and b represent an integer of 4, x represent an integer of 2, and a+b+x=10.

Specific examples of the unsubstituted aromatic hydrocarbon group having 6 to 18 carbon atoms, the unsubstituted aromatic heterocyclic group having 3 to 17 carbon atoms, or the linked aromatic group in which two to five aromatic rings of these aromatic groups are linked to each other for Ar¹ include a group generated by removing one hydrogen from benzene, naphthalene, acenaphthene, acenaphthylene, azulene, anthracene, chrysene, pyrene, phenanthrene, fluorene, triphenylene, pyridine, pyrimidine, triazine, thiophene, isothiazole, thiazole, pyridazine, pyrrole, pyrazole, imidazole, triazole, pyrazine, furan, isoxazole, quinoline, isoquinoline, quinoxaline, quinazoline, thiadiazole, phthalazine, tetrazole, indole, benzofuran, benzothiophene, benzoxazole, benzothiazole, indazole, benzimidazole, benzotriazole, benzoisothiazole, benzothiadiazole, purine, pyranone, coumarin, isocoumarin, chromone, dibenzofuran, dibenzothiophene, dibenzoselenophene, carbazole, or compounds in which two to five of these are linked to each other. Preferred examples thereof include a group generated from benzene, naphthalene, acenaphthene, acenaphthylene, azulene, anthracene, chrysene, pyrene, phenanthrene, fluorene, triphenylene, or compounds in which two to five of these are linked to each other. More preferred is a phenyl group, a biphenyl group, or a terphenyl group. The terphenyl group may be linked linearly or branched. The mode of linking may be any of ortho, meta, or para position, and it is preferable to contain ortho-linkage.

In Ar¹, each of the aromatic hydrocarbon group, the aromatic heterocyclic group, or the linked aromatic group may have a substituent. In the case that a substituent is contained, the substituent is preferably a halogen, a cyano group, a triarylsilyl group, an aliphatic hydrocarbon group having 1 to 10 carbon atoms, an alkenyl group having 2 to 5 carbon atoms, an alkoxy group having 1 to 5 carbon atoms, or a diarylamino group having 12 to 44 carbon atoms. When the substituent is an aliphatic hydrocarbon group having 1 to 10 carbon atoms, the substituent may be linear, branched, or cyclic. When the triarylsilyl group or the diarylamino group substitutes the aromatic hydrocarbon group, the aromatic heterocyclic group, or the linked aromatic group, silicon and carbon or nitrogen and carbon are bonded together by a single bond.

The number of substituents is preferably 0 to 5, and more preferably 0 to 2. In calculating the number of carbon atoms when the aromatic hydrocarbon group and the aromatic heterocyclic group have a substituent, the number of carbon atoms of the substituent is not included. However, it is preferable that the total number of carbon atoms including the number of carbon atoms of the substituent satisfy the above range.

Specific examples of the substituent include cyano, methyl, ethyl, propyl, i-propyl, butyl, t-butyl, pentyl, neopentyl, cyclopentyl, hexyl, cyclohexyl, heptyl, octyl, nonyl, decyl, vinyl, propenyl, butenyl, pentenyl, methoxy, ethoxy, propoxy, butoxy, pentoxy, diphenylamino, naphthylphenylamino, dinaphthylamino, dianthranilamino, diphenanthrenylamino, and dipyrenylamino. Preferred examples thereof include cyano, methyl, ethyl, t-butyl, propyl, butyl, pentyl, neopentyl, hexyl, heptyl, or octyldiphenylamino, naphthylphenylamino, or dinaphthylamino.

Examples of the aliphatic hydrocarbon group having 1 to 10 carbon atoms include methyl, ethyl, propyl, butyl, pentyl, hexyl, heptyl, octyl, nonyl, and decyl. Preferred is an alkyl group having 1 to 4 carbon atoms.

The linked aromatic group refers to an aromatic group in which the aromatic rings in two or more aromatic groups are linked to each other by a single bond. The linked aromatic group may be linear or branched. The linkage position in linking of benzene rings may be any of the ortho-, meta-, and para-positions, and is preferably the para-position or the meta-position. The aromatic group may be an aromatic hydrocarbon group or an aromatic heterocyclic group, and the plurality of aromatic groups may be the same or different.

The above host material is used as a host material of the light emitting layer of an organic EL device. The host material may be one kind, and is preferably two or more kinds. When two or more kinds of host materials are used, it is preferable that the above host material be contained as a first host material and a material selected from the compound represented by the general formula (6) as a second host material.

In the general formula (6), the two carbazole rings can be bonded at the 2-position, the 3-position, or the 4-position, but preferred is bonding at the 3-position as shown in the formula (7).

In the general formula (6) and the formula (7), the same symbols have the same meaning.

In the general formula (6), Ar² and Ar³ each independently represent a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five of these aromatic groups are linked to each other. Preferred is a substituted or unsubstituted aromatic hydrocarbon group having 6 to 18 carbon atoms or a linked aromatic group in which two to three aromatic rings of the aromatic hydrocarbon groups are linked to each other, and more preferred is a substituted or unsubstituted phenyl group, a substituted or unsubstituted biphenyl group, or a substituted or unsubstituted terphenyl group. The terphenyl group may be linked linearly or branched.

L each independently represents a single bond, a substituted or unsubstituted aromatic hydrocarbon group having 6 to 18 carbon atoms, or a substituted or unsubstituted aromatic heterocyclic group having 3 to 17 carbon atoms. Preferably, L independently represents a single bond or a substituted or unsubstituted phenylene group. The mode of linking may be any of ortho-, meta-, or para-linkage.

R each independently represents deuterium or an aliphatic hydrocarbon group having 1 to 10 carbon atoms. Preferably, R each independently represents deuterium.

g to j and p to s represent the number of substitutions, g, h, r, and s each independently represent an integer of 0 to 4, i and j each independently represent an integer of 0 to 3, p and q each independently represent an integer of 0 to 13, and when L is a single bond, r and s represent an integer of 0. g+h+i+j is preferably an integer of 0 or 14.

In the general formula (6), specific examples of the aliphatic hydrocarbon group having 1 to 10 carbon atoms, the unsubstituted aromatic hydrocarbon group having 6 to 18 carbon atoms, the unsubstituted aromatic heterocyclic group having 3 to 17 carbon atoms, and the unsubstituted linked aromatic group in which two to five aromatic rings of the aromatic hydrocarbon group and the aromatic heterocyclic group are linked to each other are the same as those shown for the general formula (1). Specific examples of the substituent are the same as those shown for the general formula (1), except that deuterium is contained.

In the present invention, some or all hydrogen atoms of the unsubstituted aromatic hydrocarbon group, the unsubstituted aromatic heterocyclic group, the unsubstituted linked aromatic group, the substituents of these aromatic groups, or the aliphatic hydrocarbon group may be deuterated. However, as described above, the substituent substituted with triazine, and Ar¹ are not deuterated in the general formula (1), and some or all of hydrogen in the compounds represented by the general formulas (1) to (7) may be deuterium. A deuterated product encompasses both the case of a single compound included and the case of a mixture of two or more compounds included. In other words, the rate of deuteration is specifically described, and means that half the total hydrogen, on average, is substituted with deuterium in the case of a rate of deuteration of 50%, in which the deuterated product may be a single compound or a mixture of those different in rate of deuteration.

When some hydrogen in the compounds represented by the general formulas (1) to (5) are deuterium, it is preferable that 10% or more of hydrogen atoms be deuterium, and it is more preferable that 20% or more and 40% or less thereof be deuterium and the rate of deuteration be such that the total number of a+b+x is 2 or more and 10 or less.

When some hydrogen in the compounds represented by the general formulas (6), (7) are deuterium, preferably 30% or more of hydrogen atoms are deuterium, more preferably 40% or more thereof are deuterium, and further preferably 50% or more thereof are deuterium.

The rate of deuteration can be determined by mass analysis or proton nuclear magnetic resonance spectroscopy. For example, when the rate of deuteration is determined by proton nuclear magnetic resonance spectroscopy, first, a measurement sample is prepared by adding and dissolving a compound and an internal standard material to and in a deuterated solvent, and the proton concentration [mol/g] in the compound included in the measurement sample is calculated from the ratio between the respective integral intensities derived from the internal standard material and the compound. Next, the ratio between the proton concentration in a deuterated compound and the proton concentration in the corresponding non-deuterated compound is calculated, and subtracted from 1, thereby enabling calculation of the rate of deuteration of the deuterated compound. The rate of deuteration of a partial structure can be calculated from the integral intensity of the chemical shift derived from an objective partial structure, according to the same procedure.

Specific examples of the compound represented by the general formula (1) are shown below, but are not limited to these exemplified compounds.

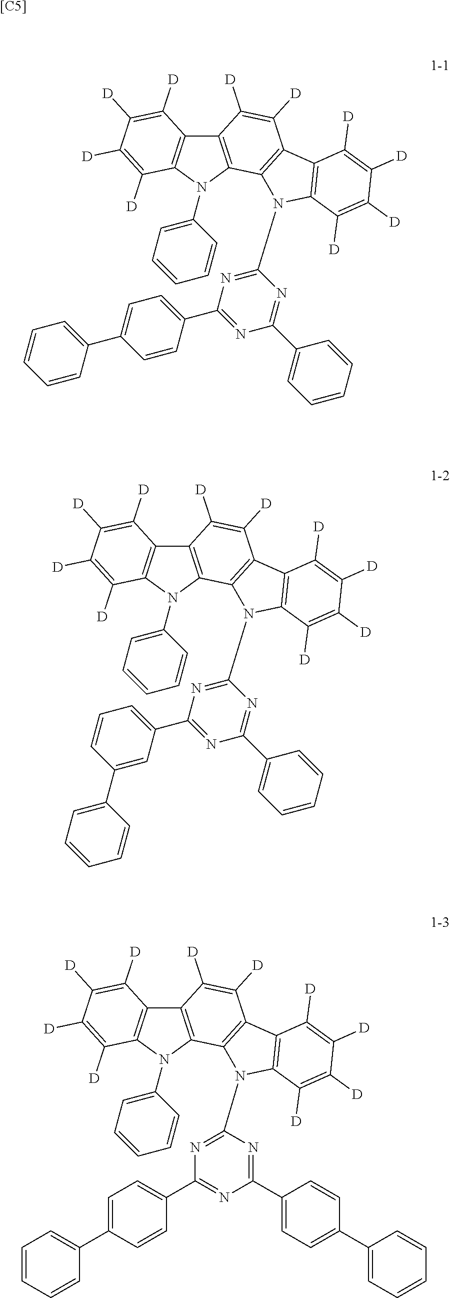

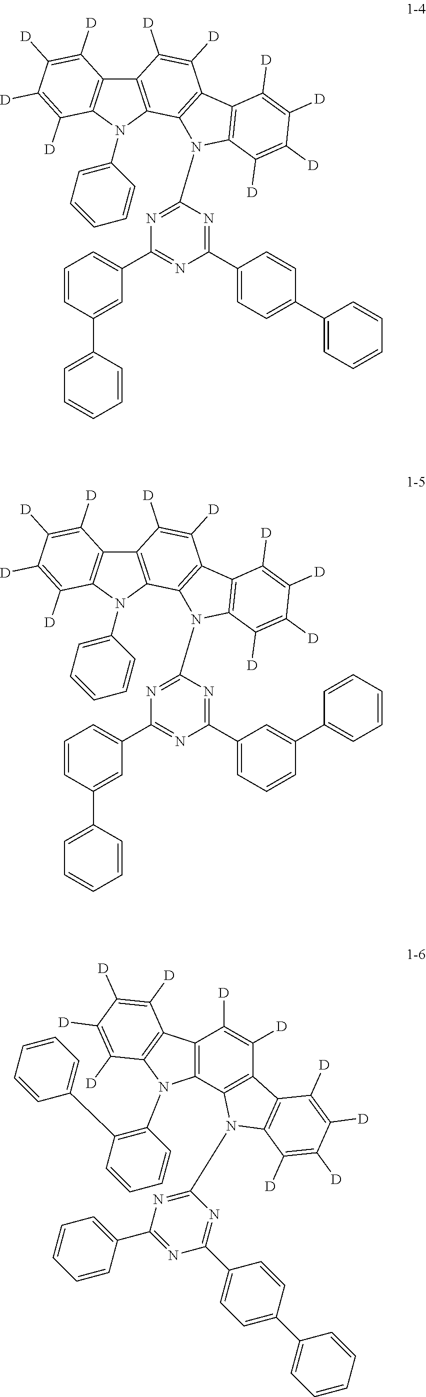

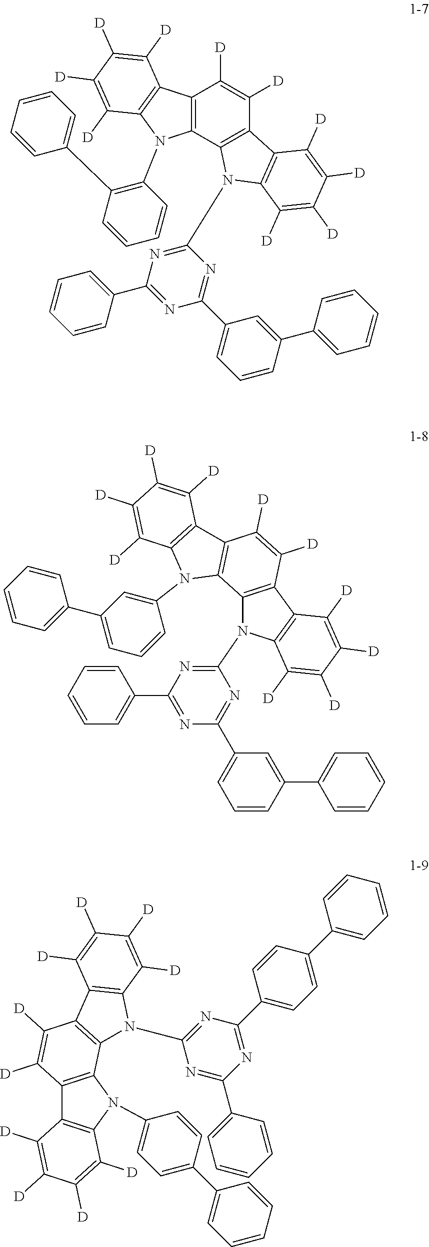

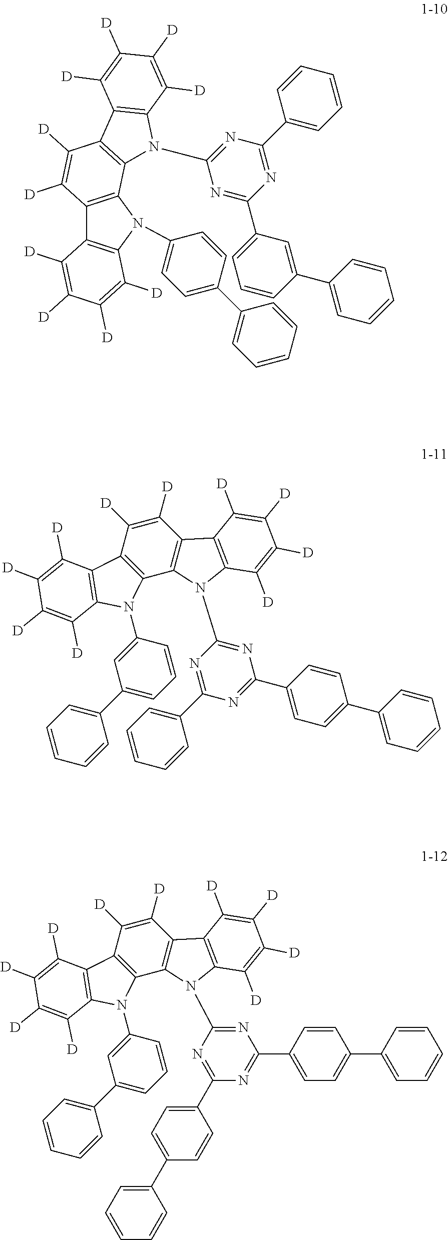

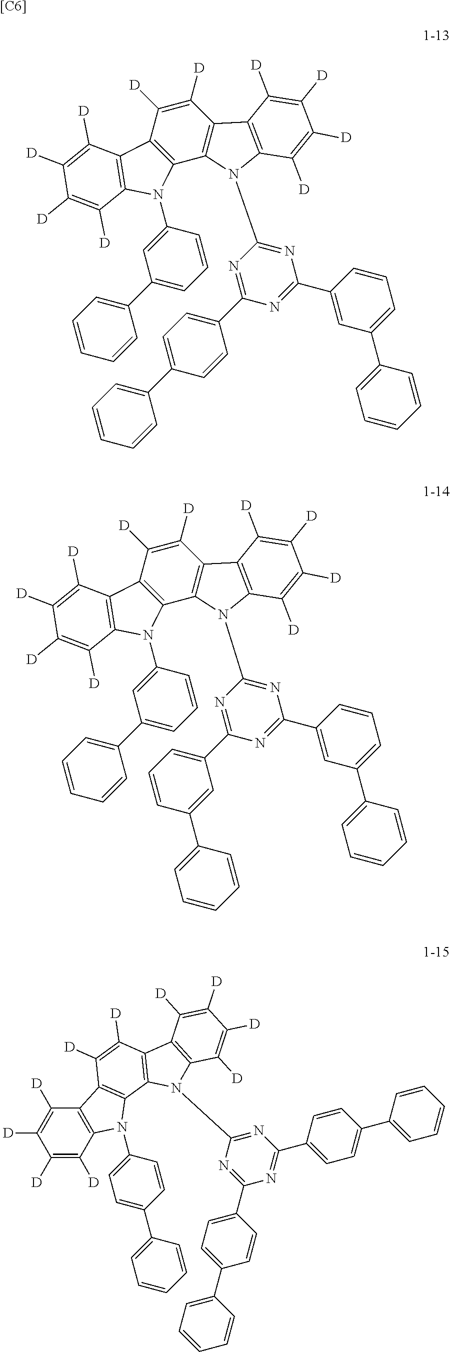

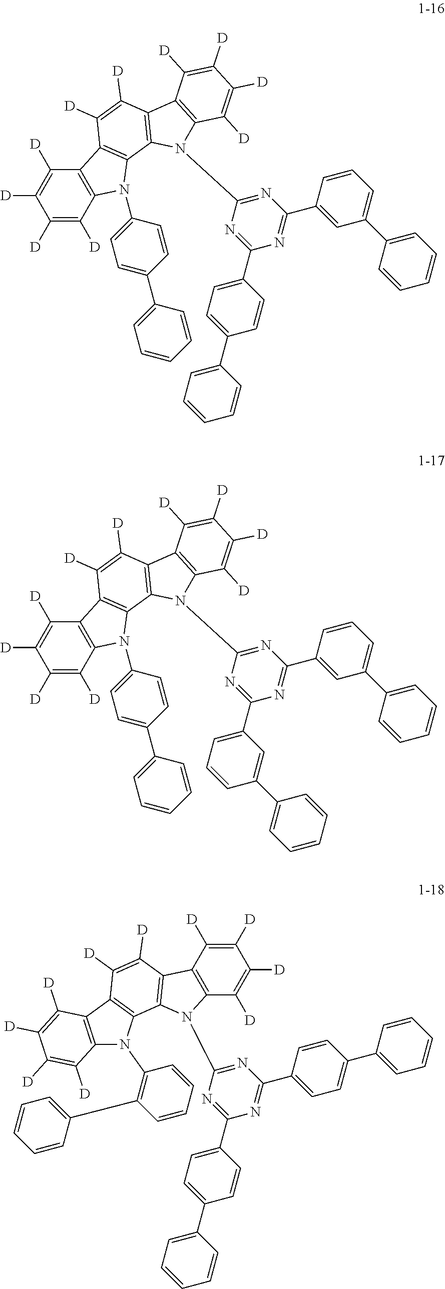

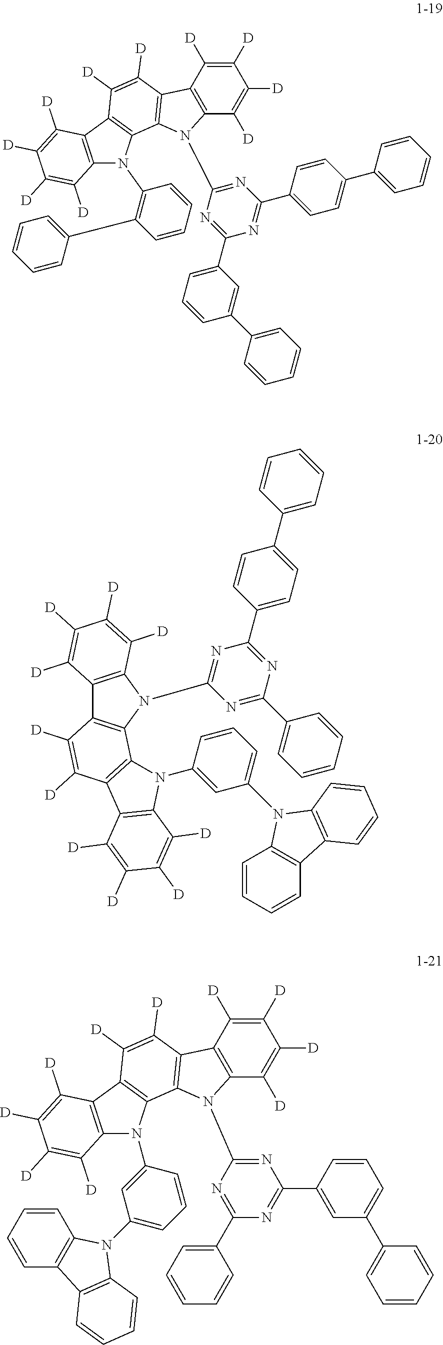

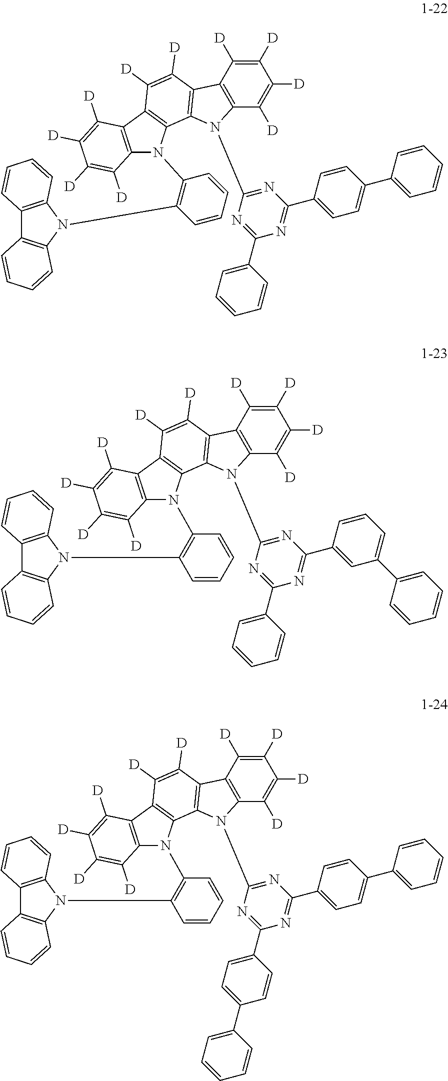

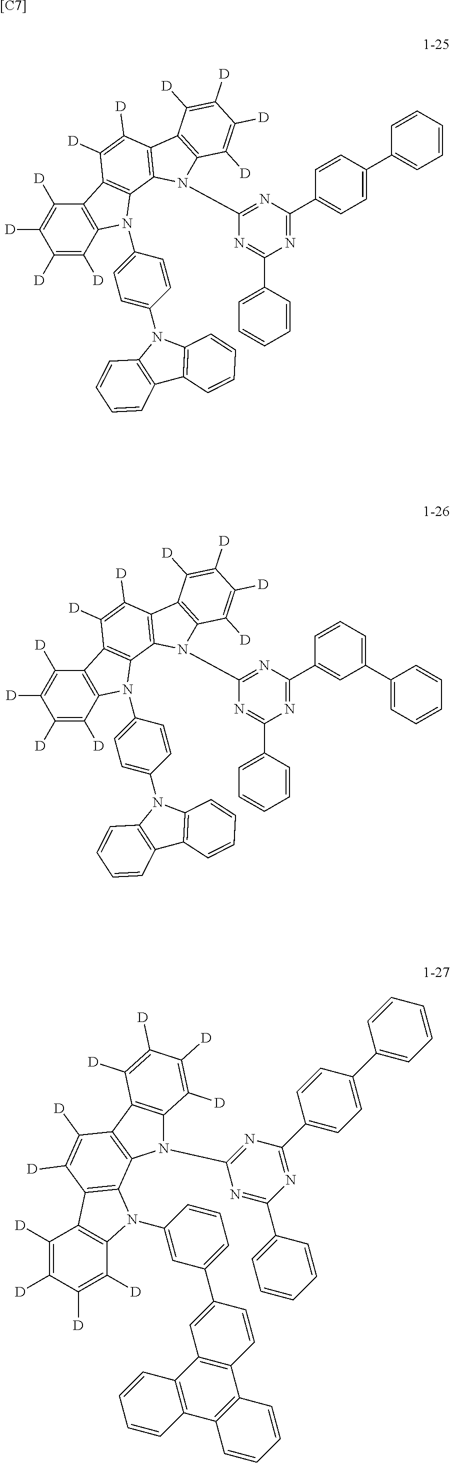

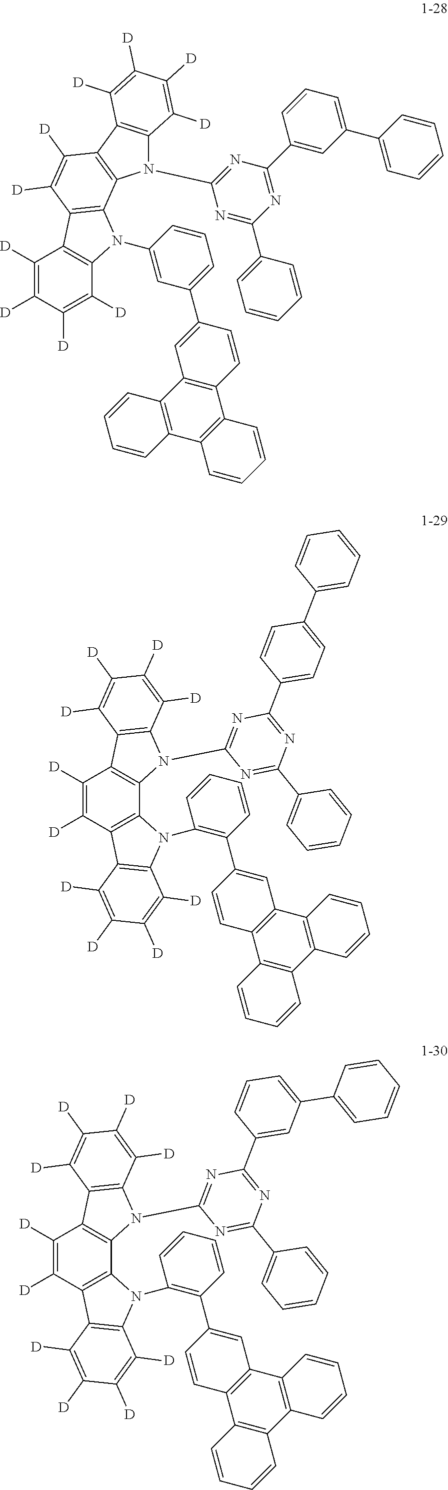

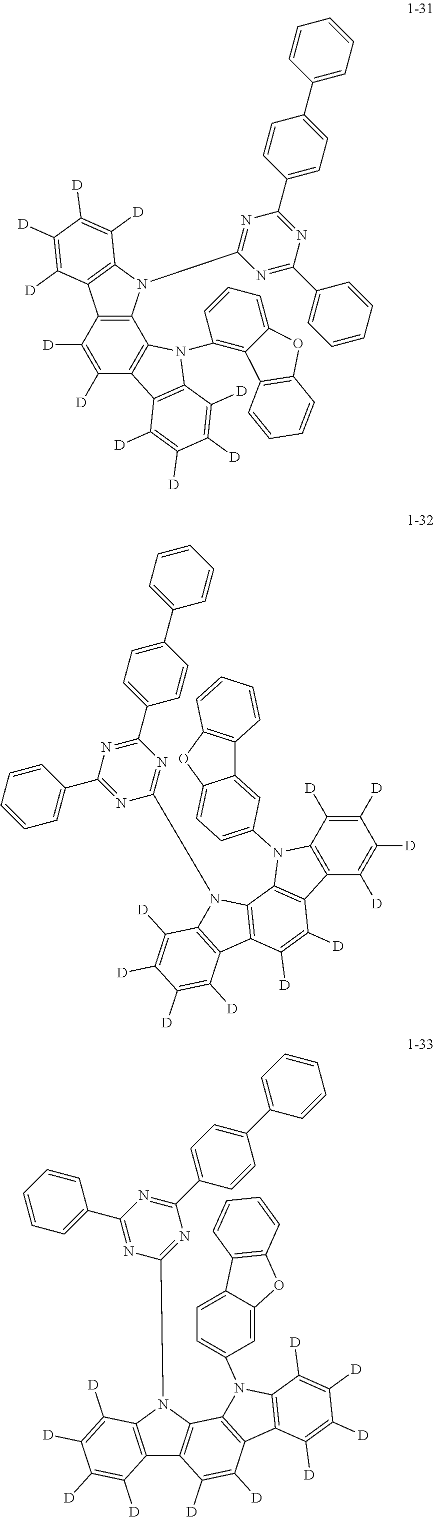

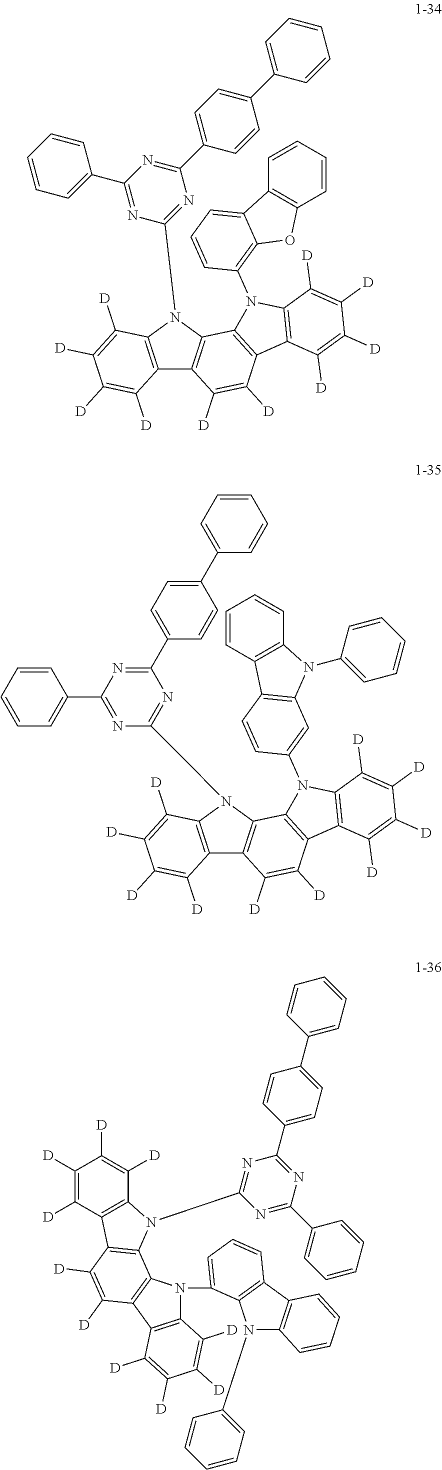

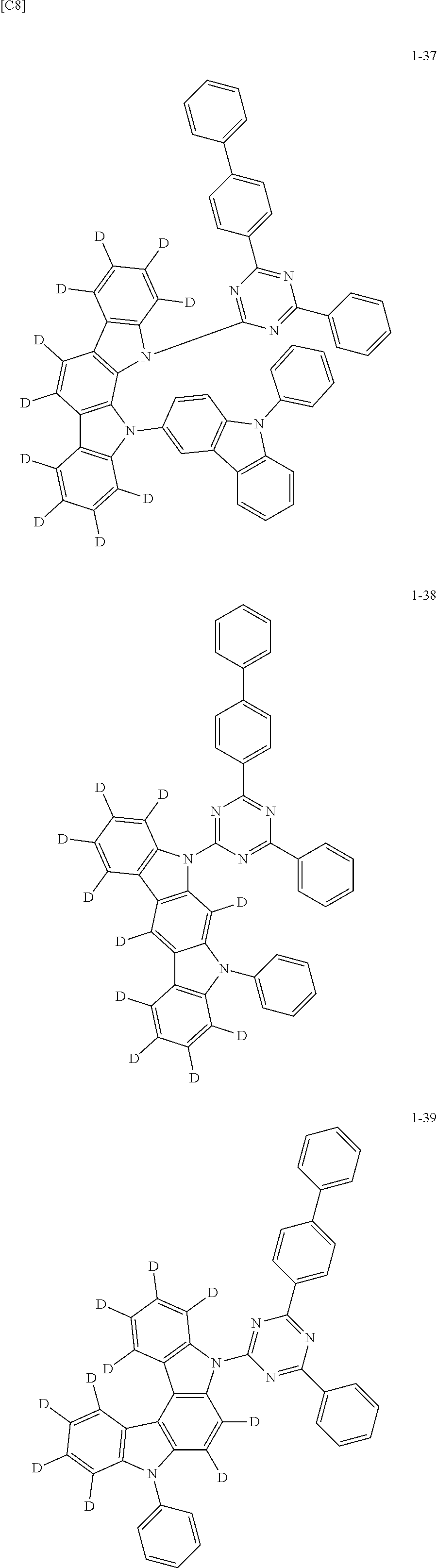

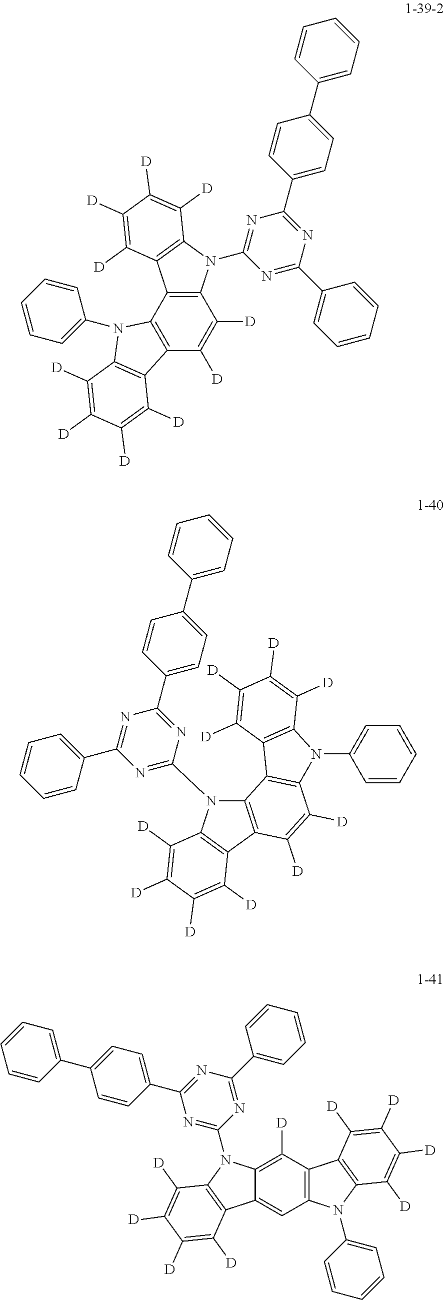

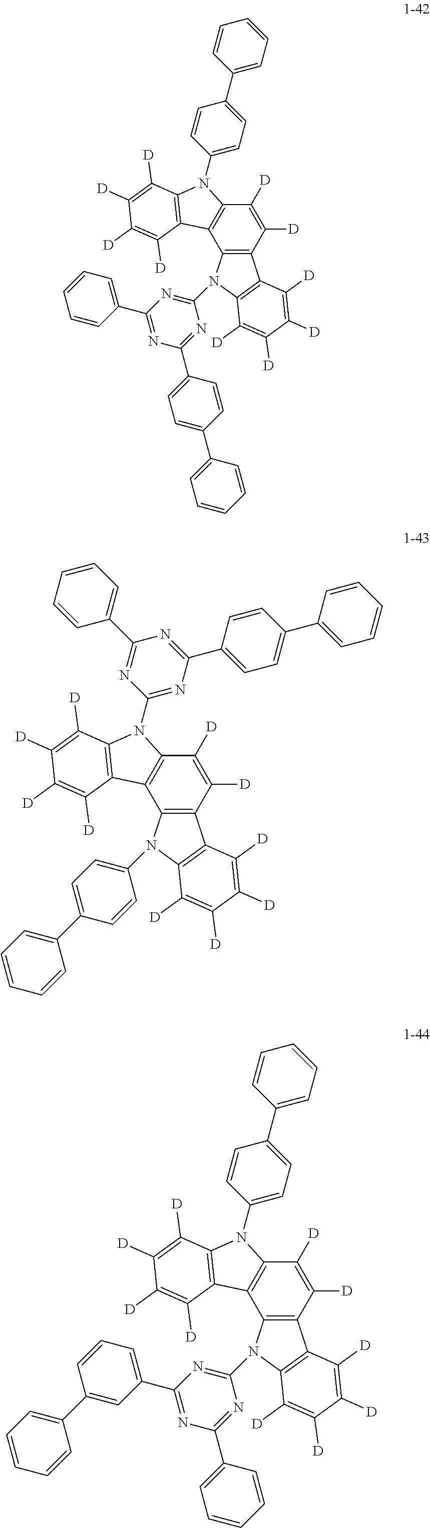

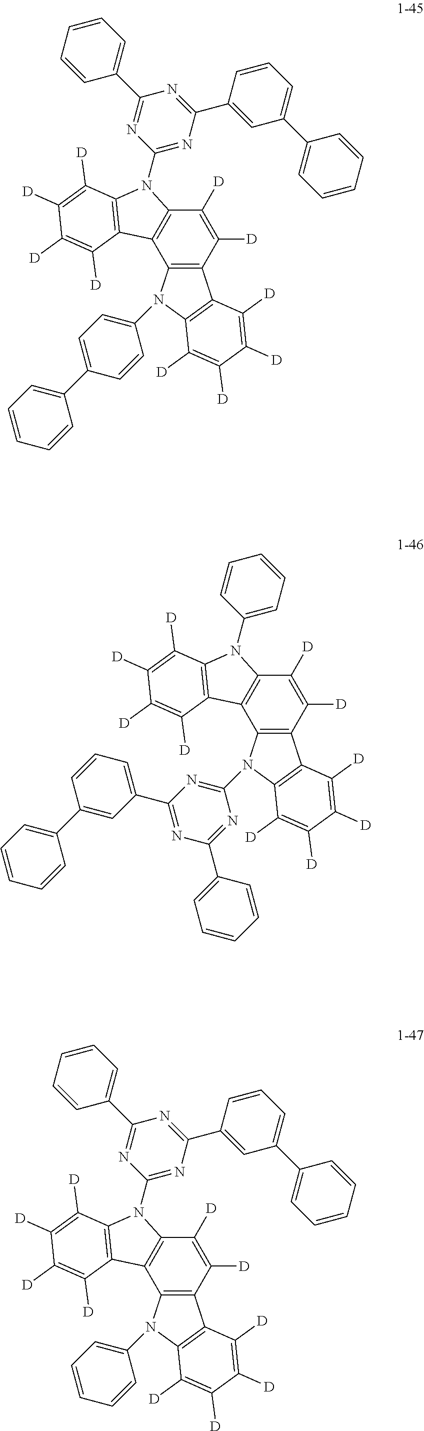

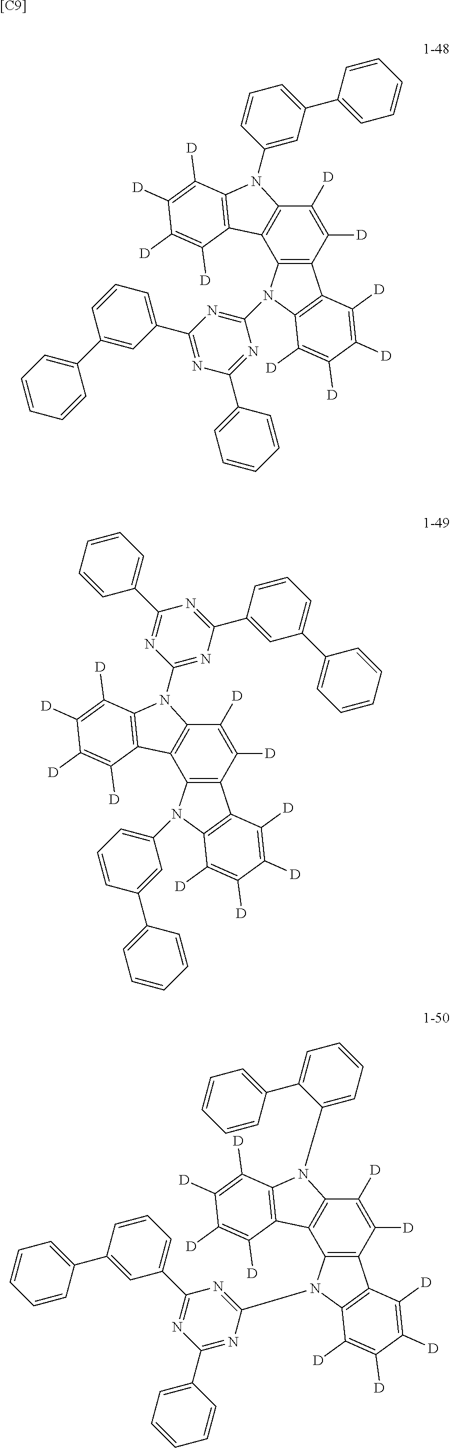

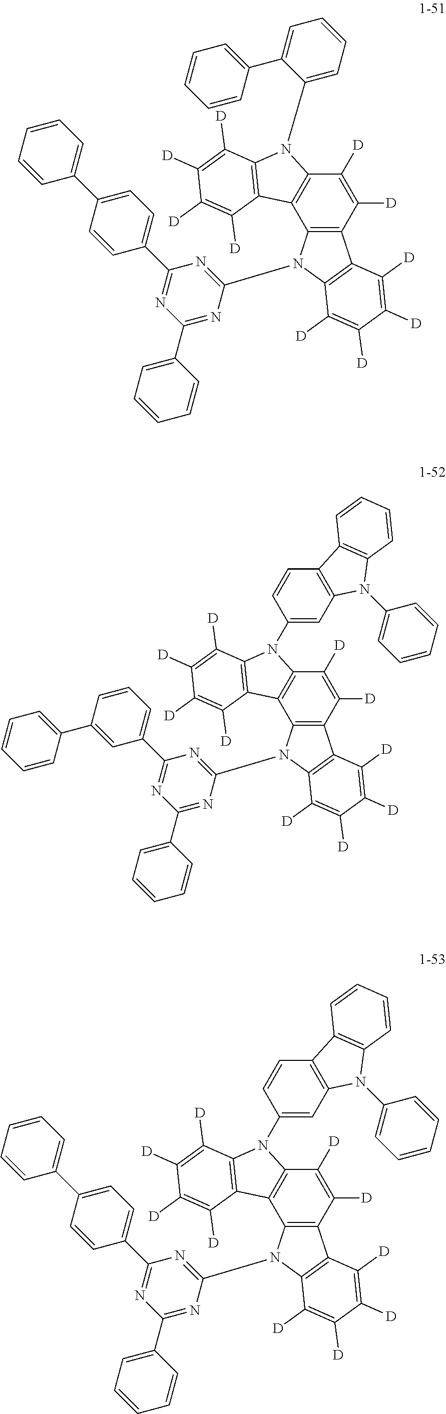

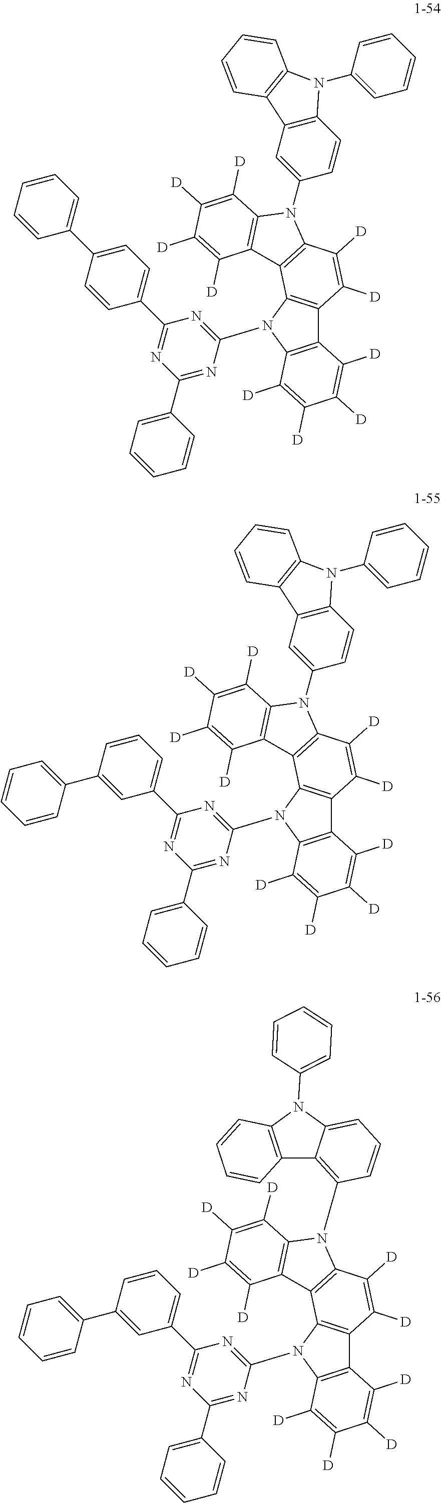

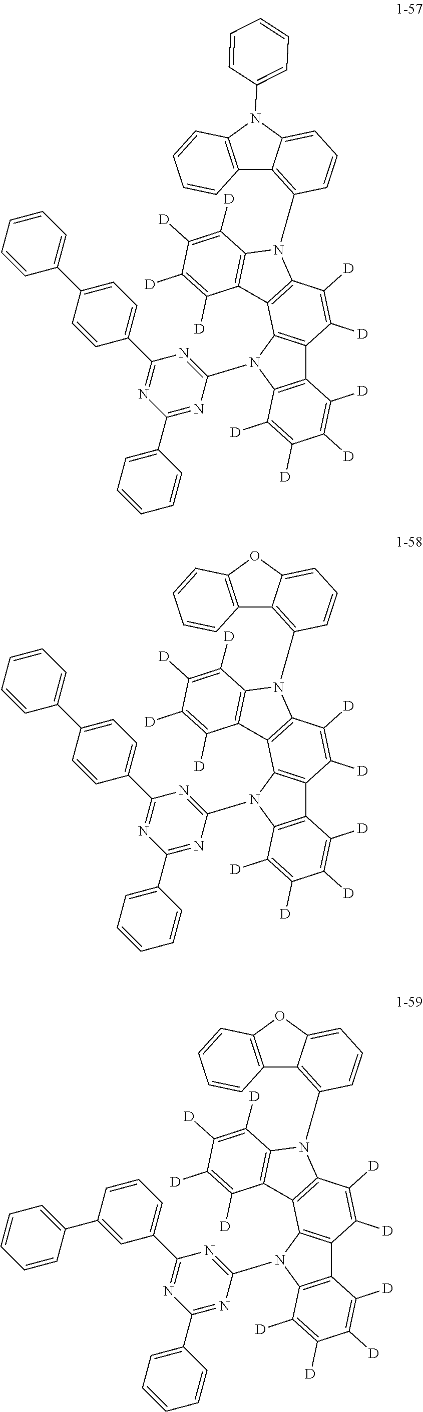

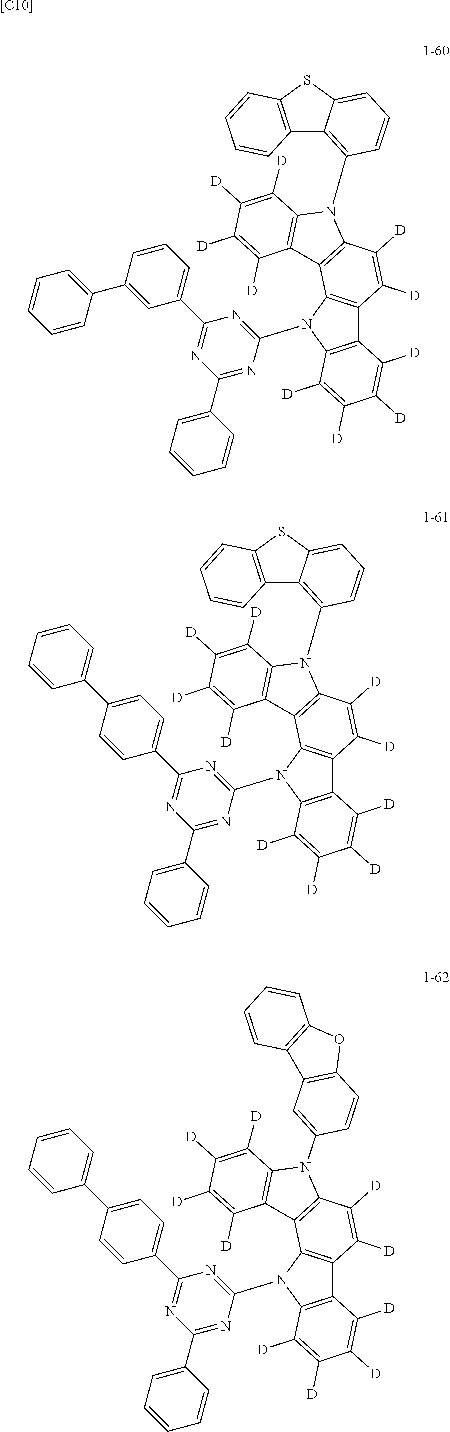

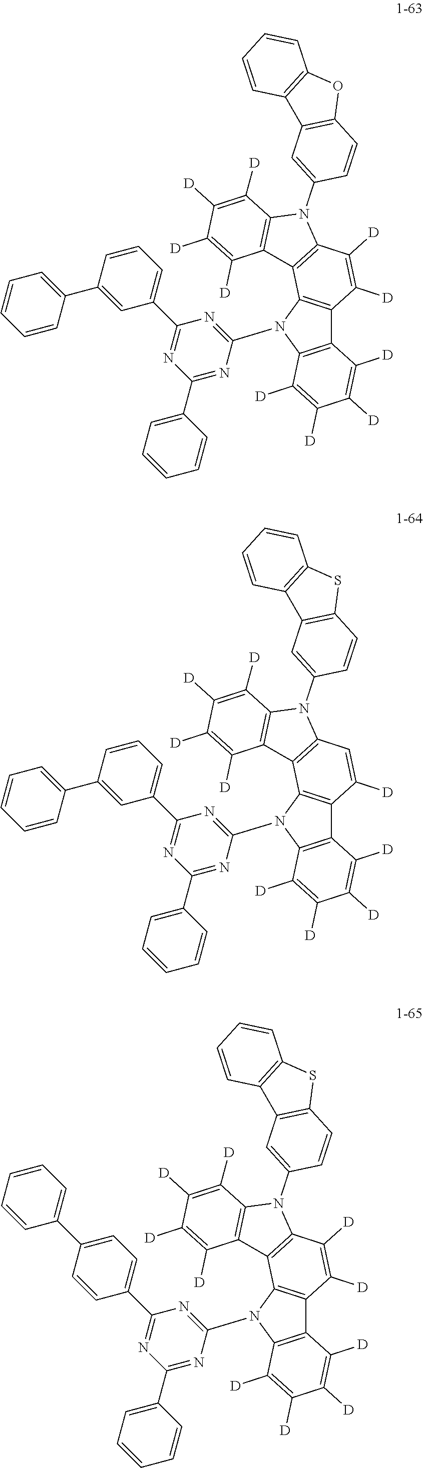

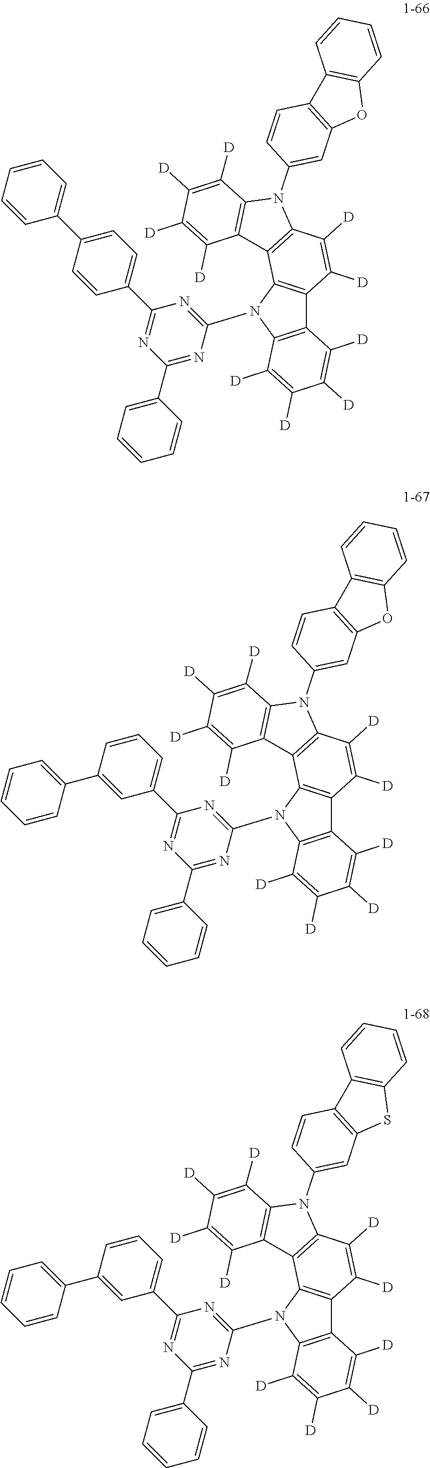

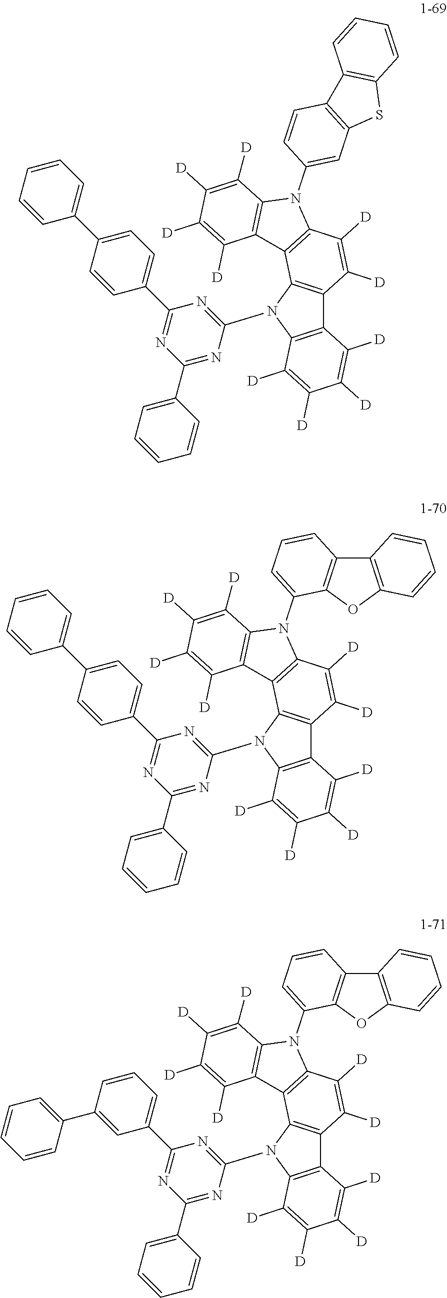

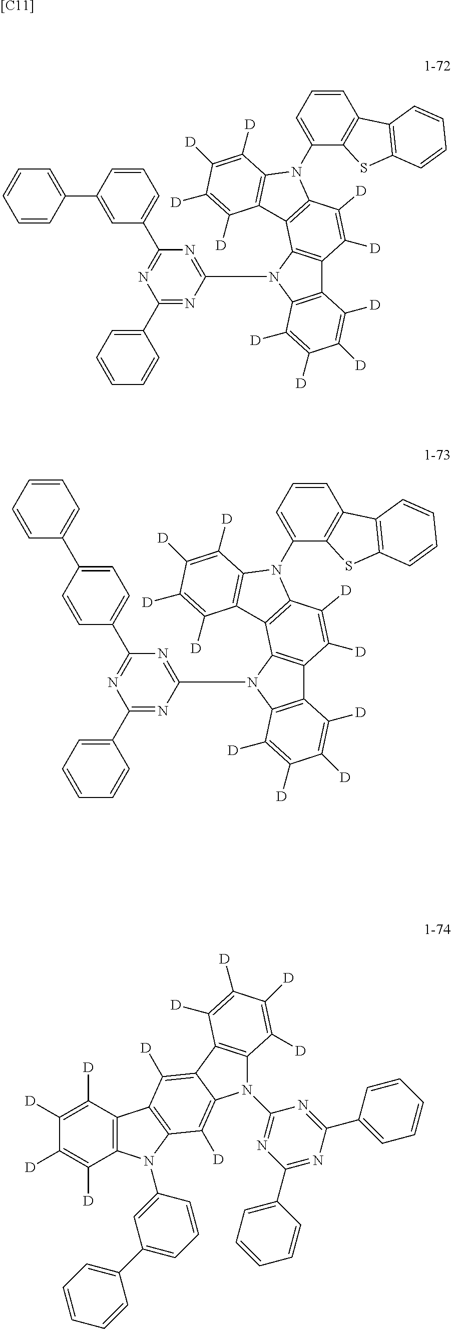

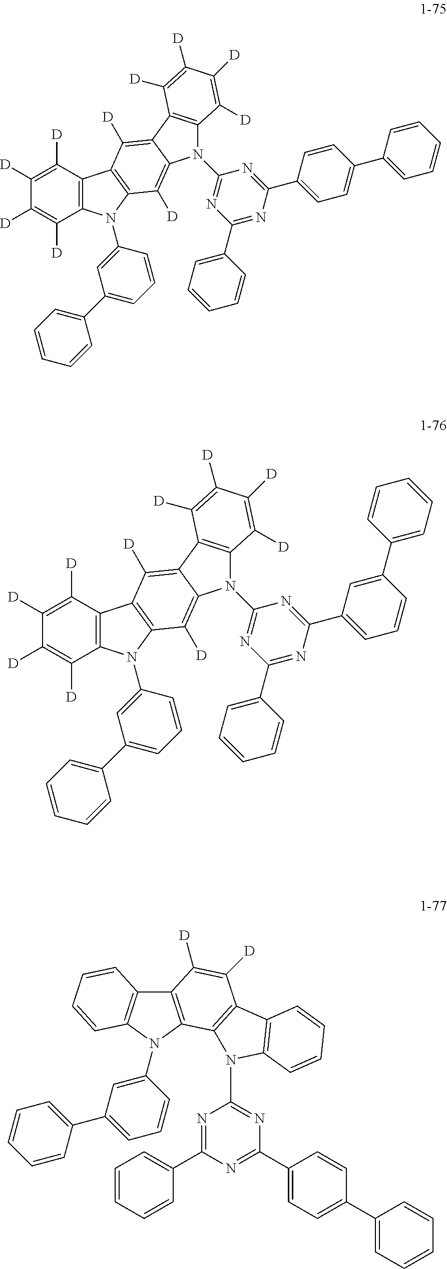

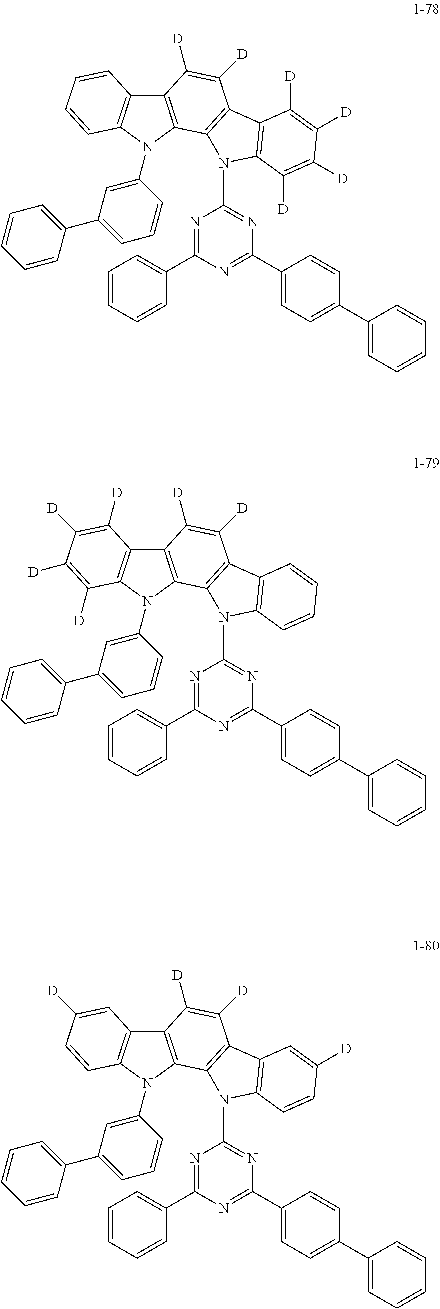

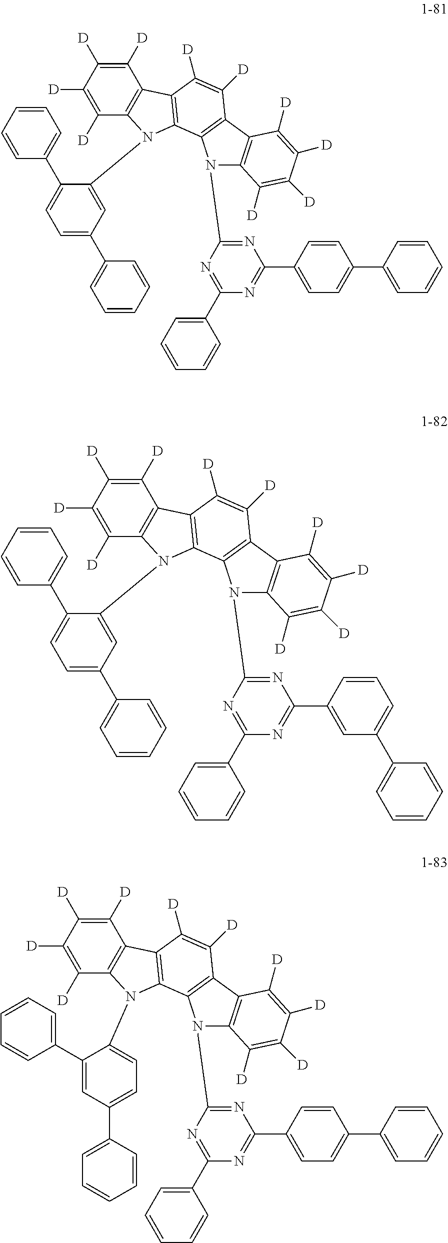

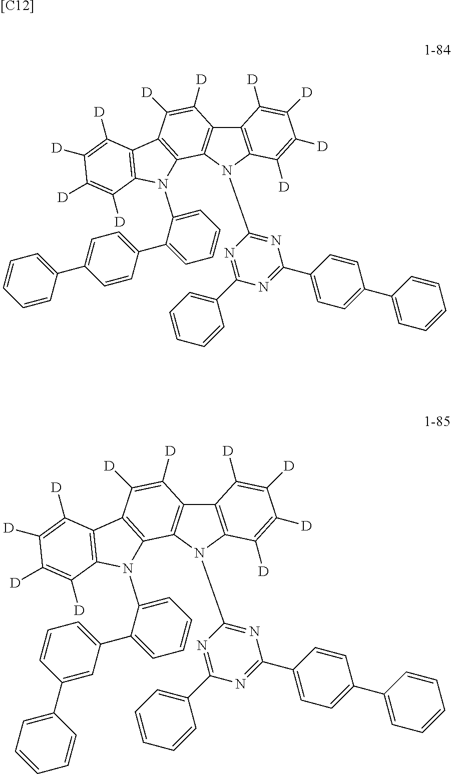

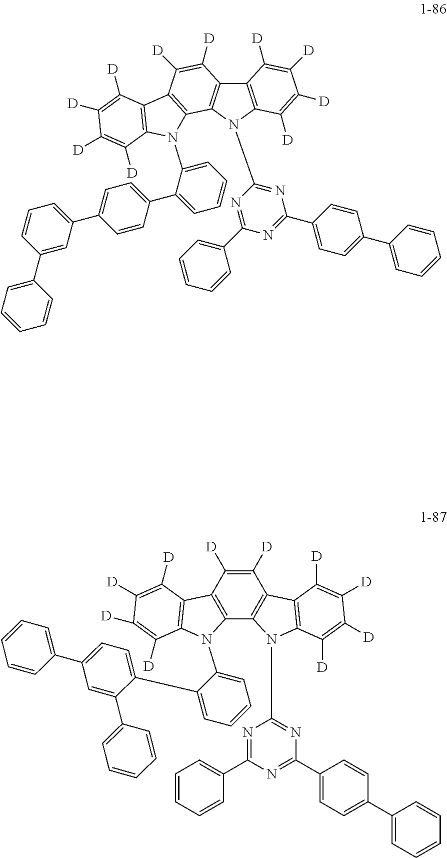

Specific examples of the compounds represented by the general formula (6) and the formula (7) are shown below, but are not limited to these exemplified compounds.

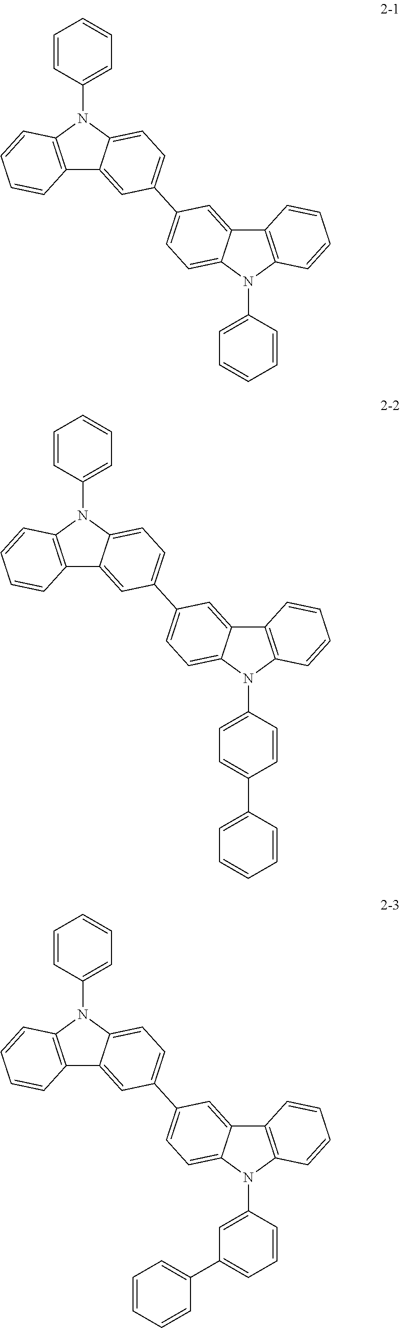

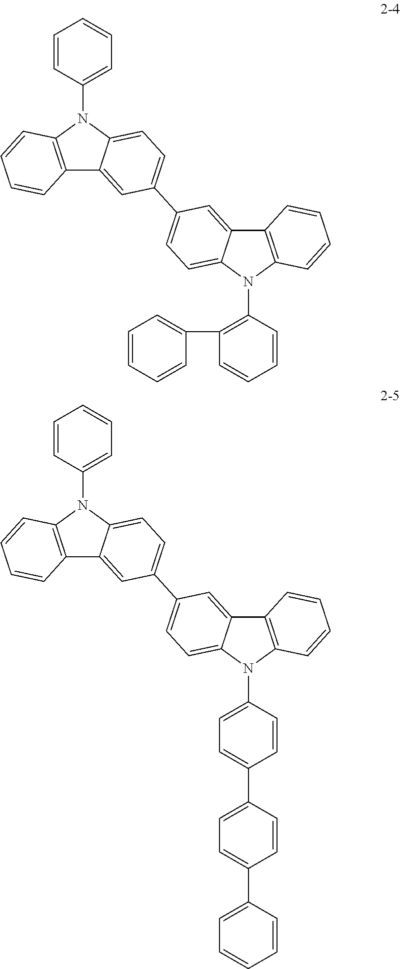

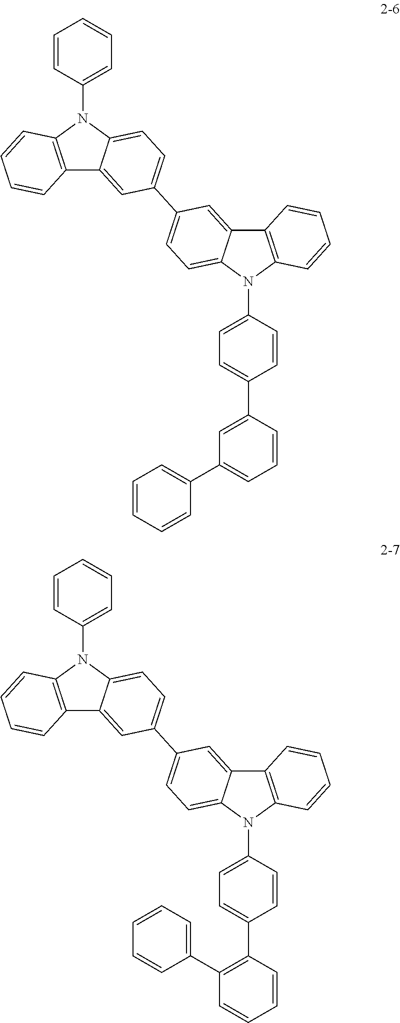

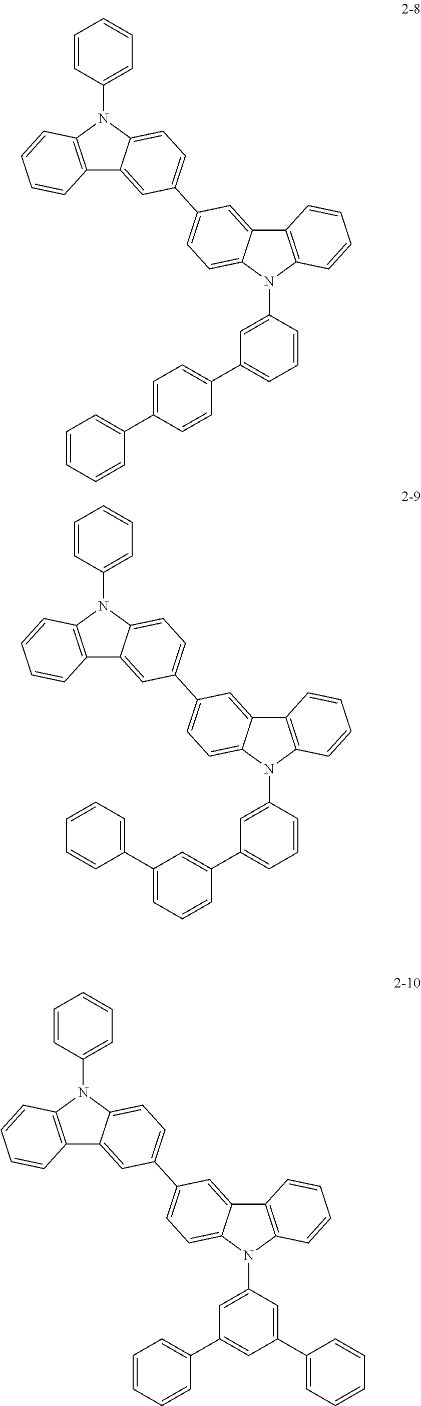

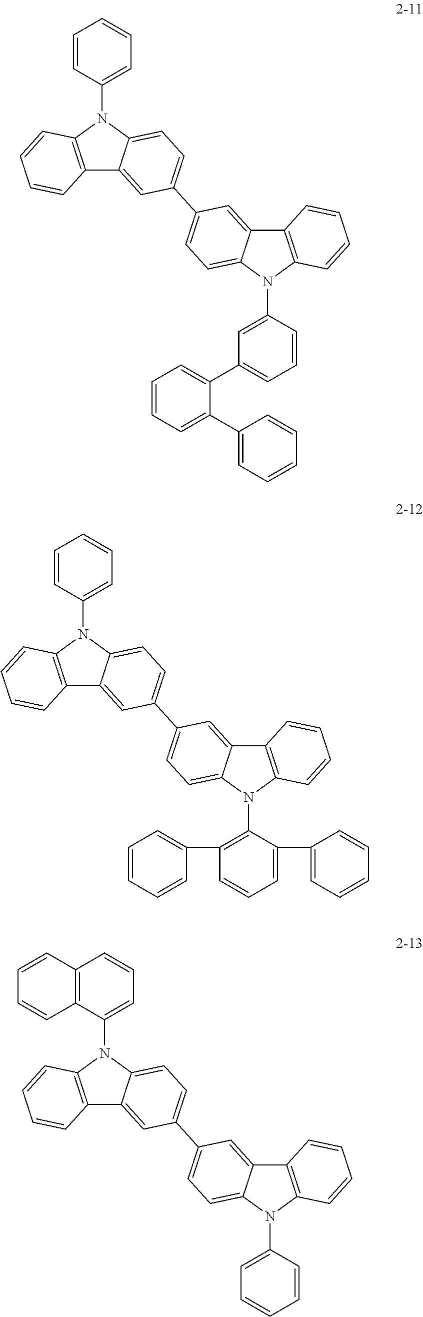

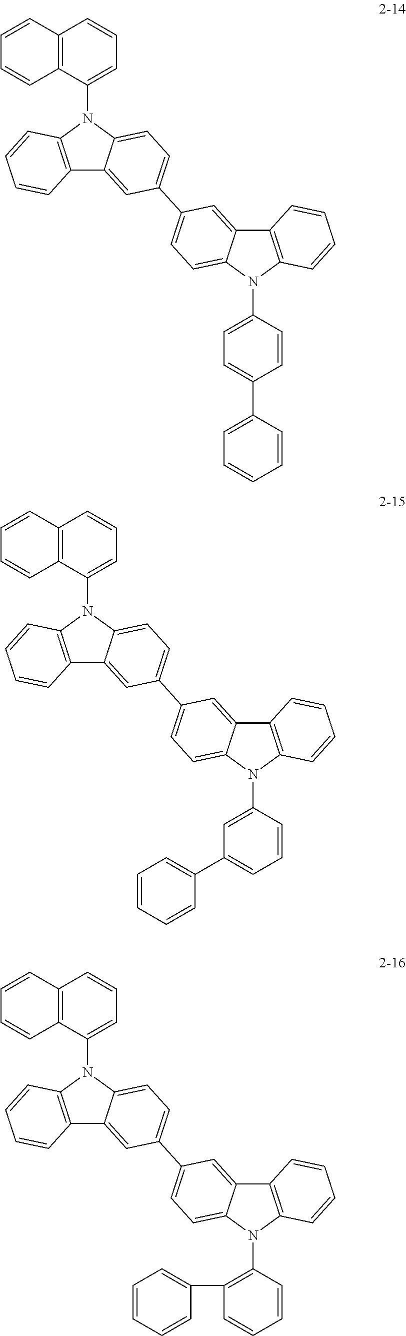

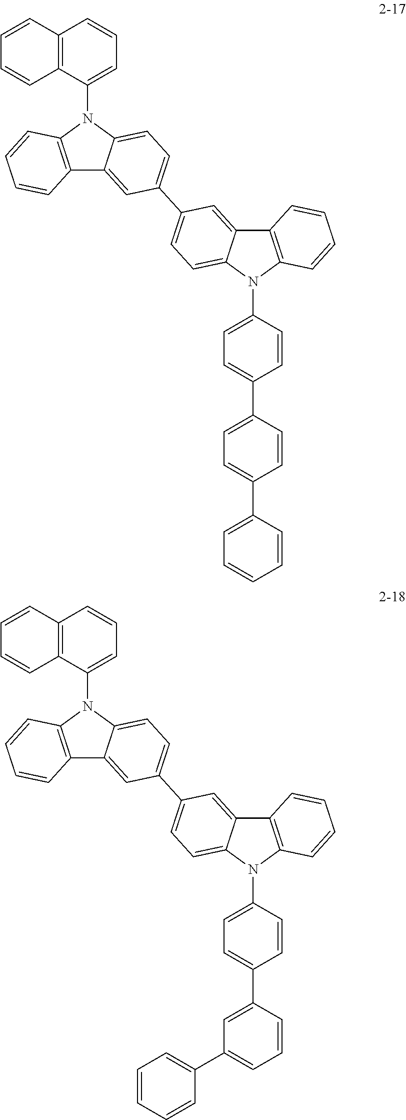

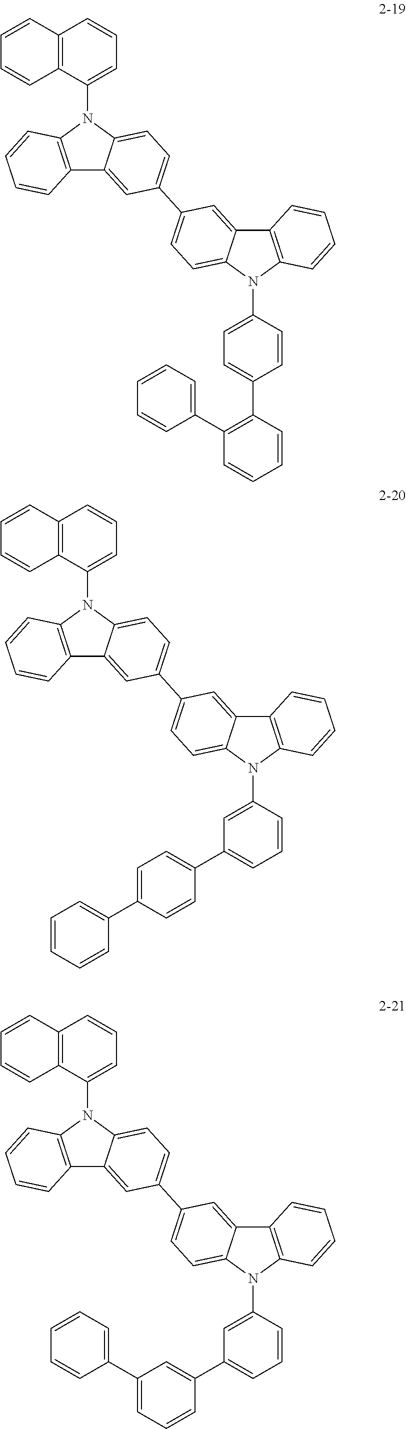

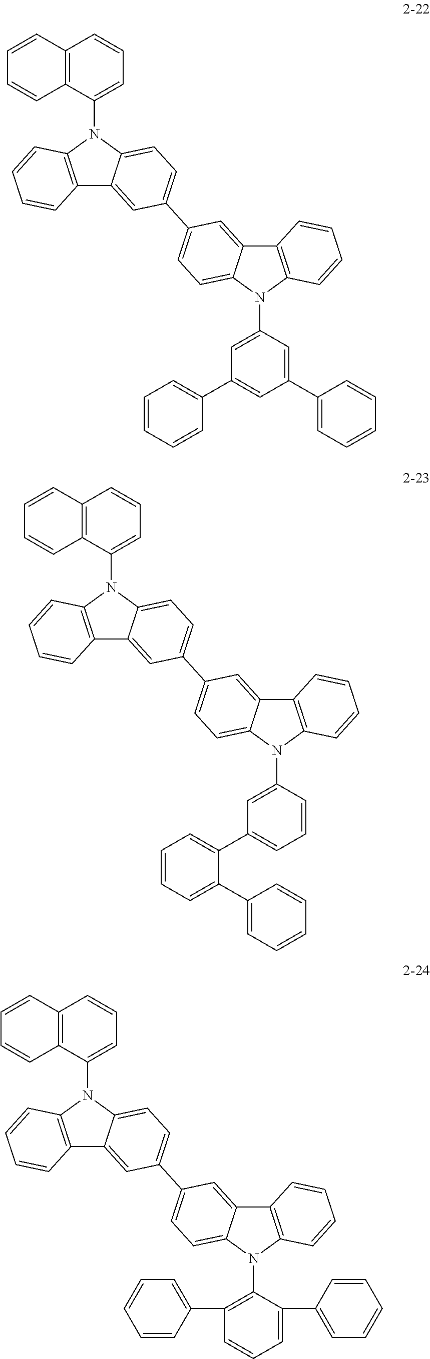

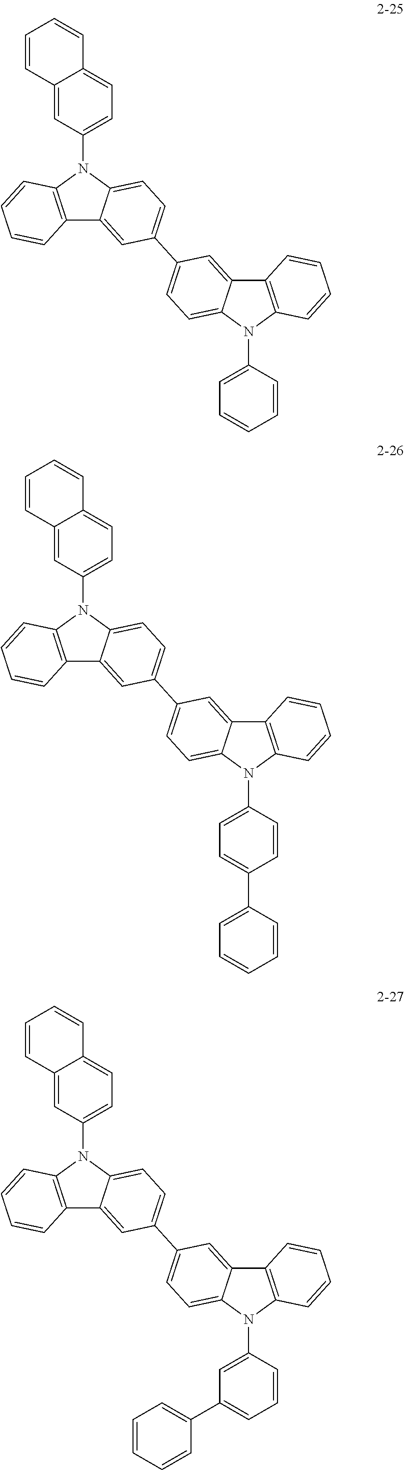

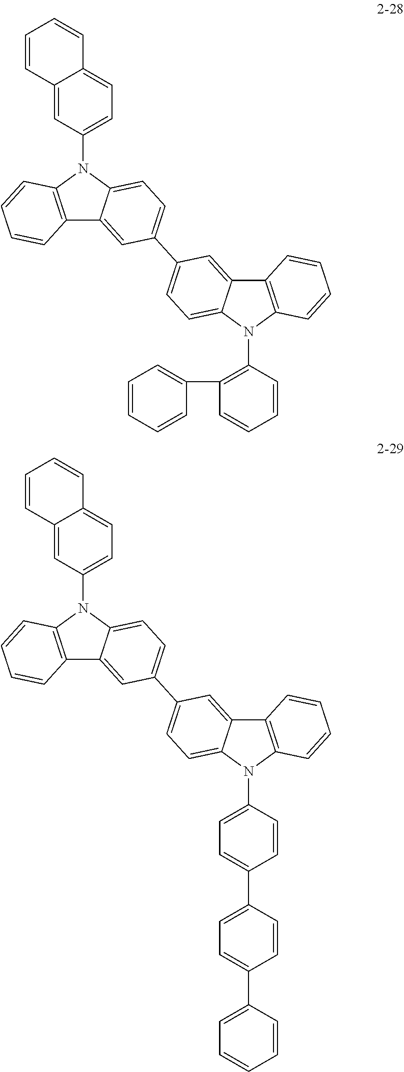

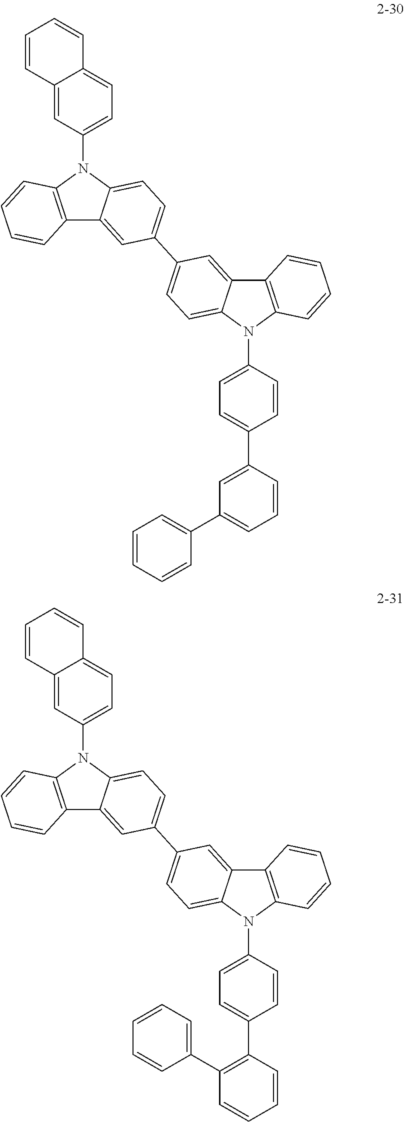

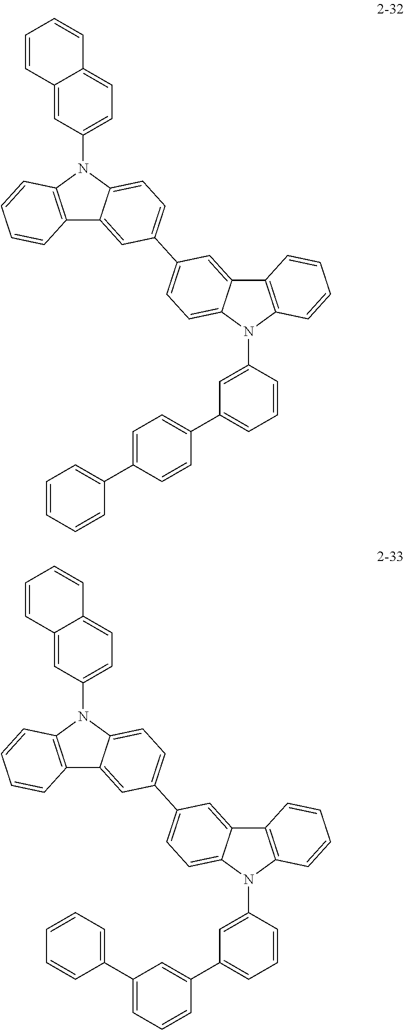

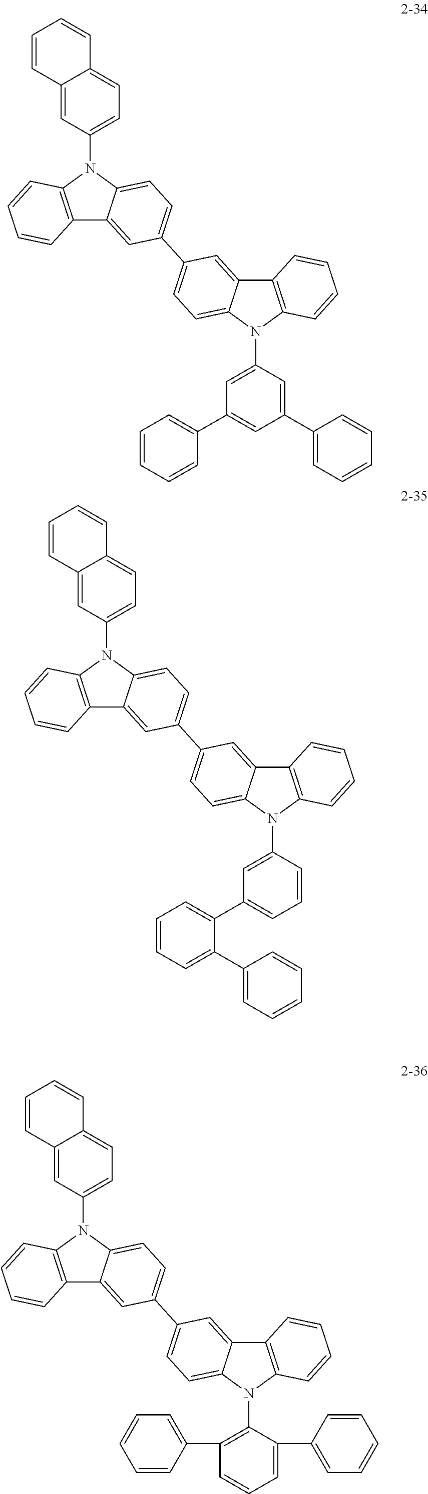

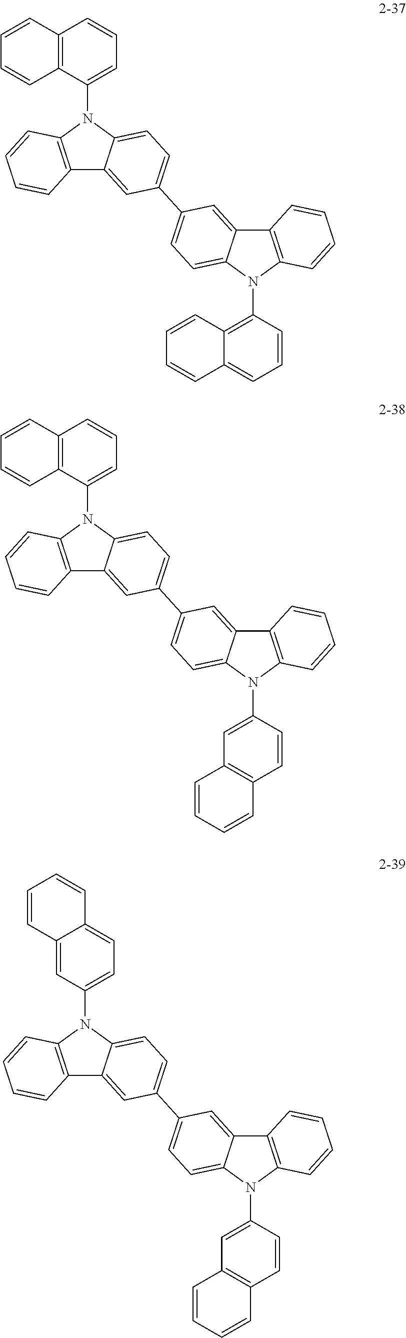

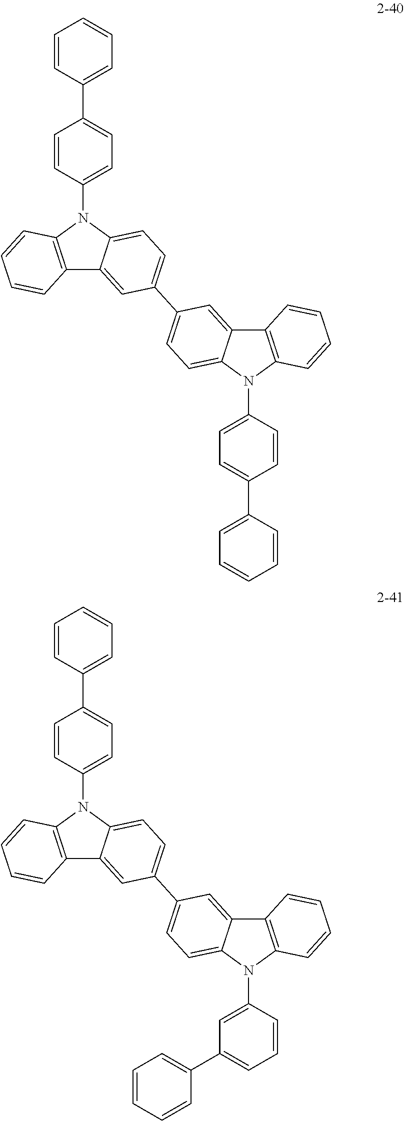

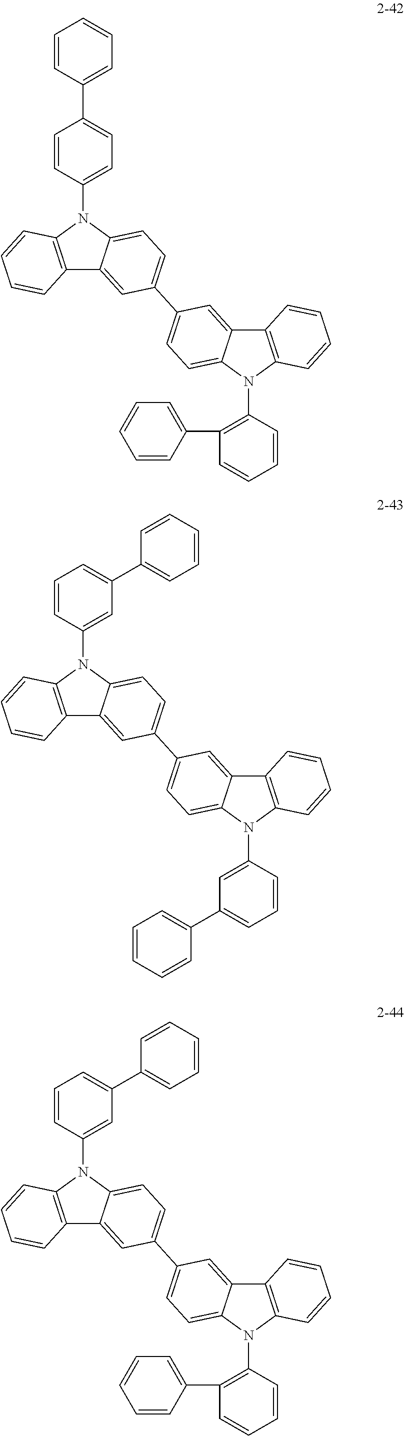

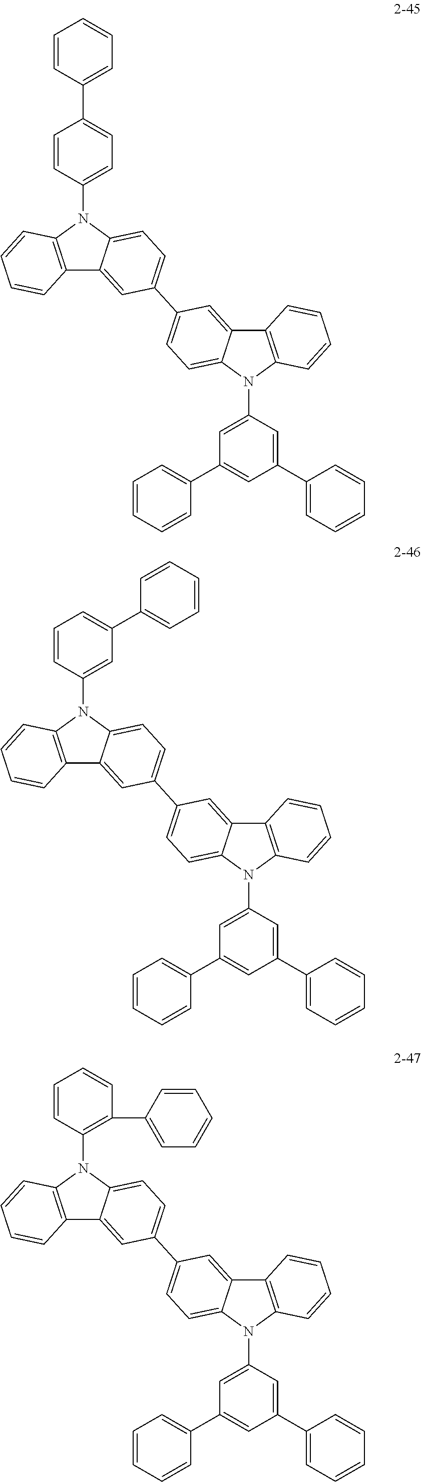

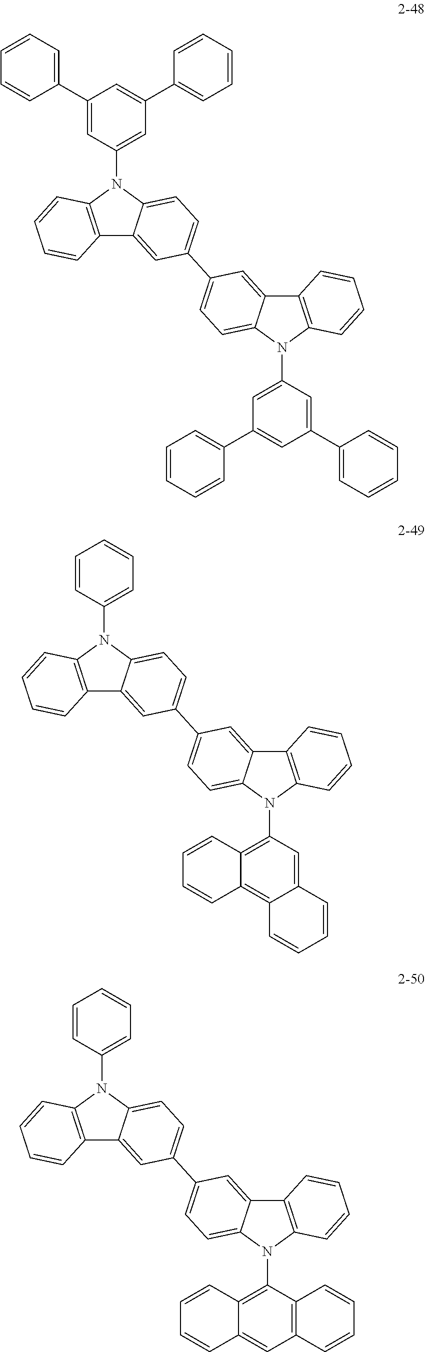

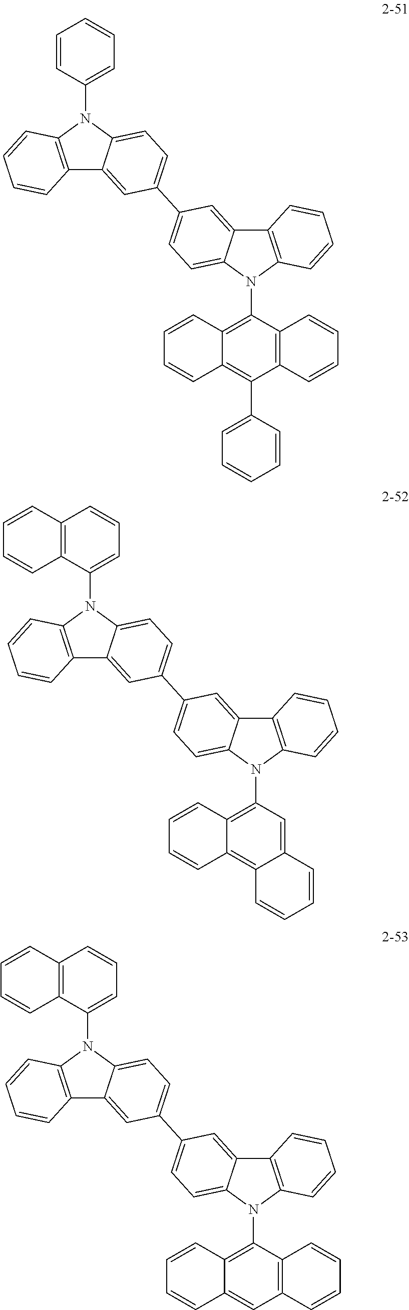

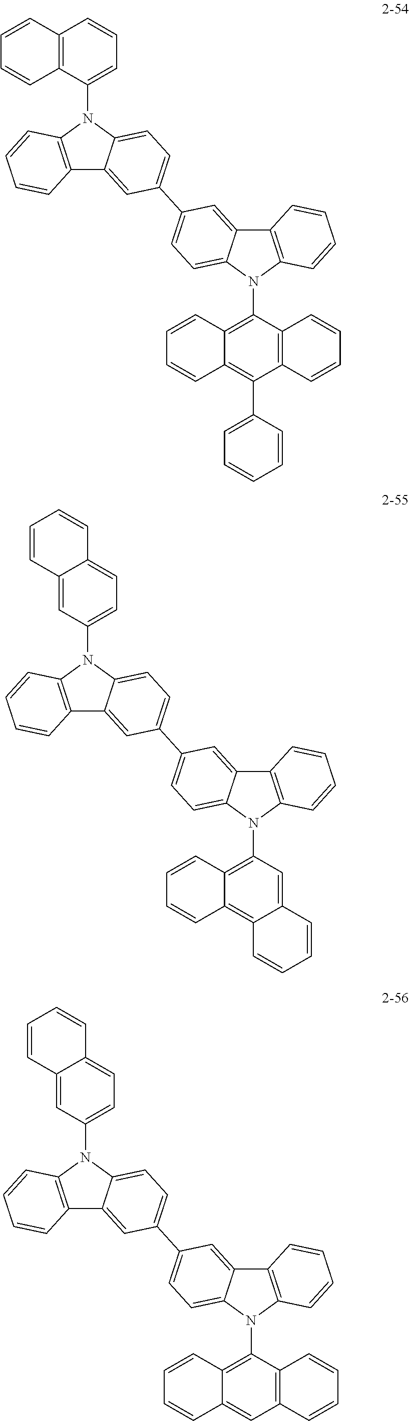

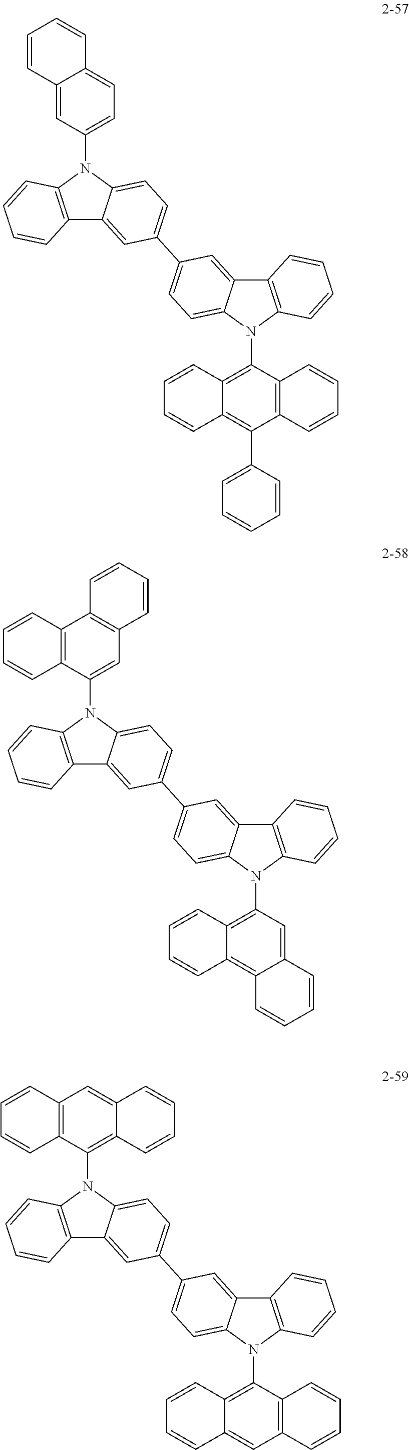

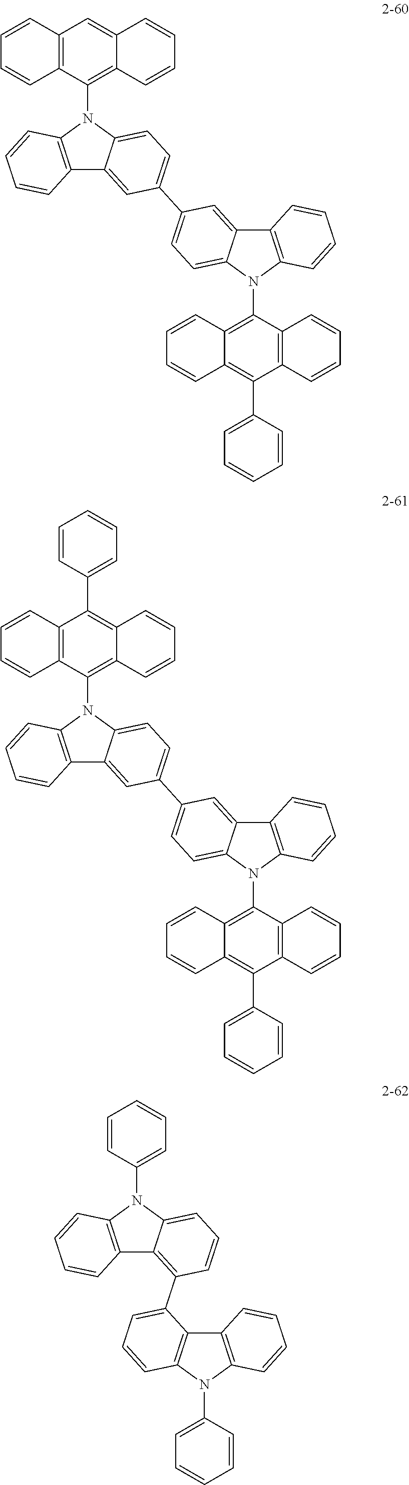

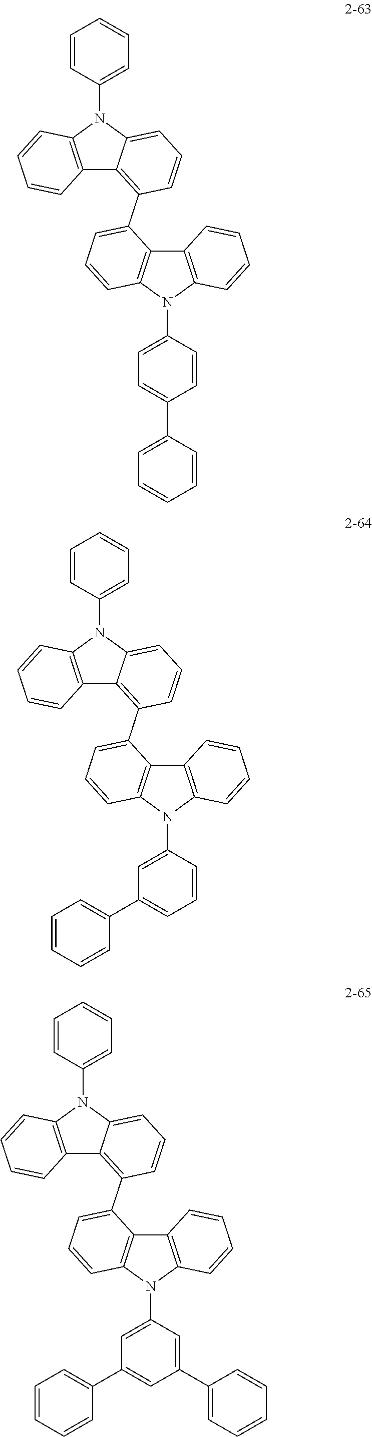

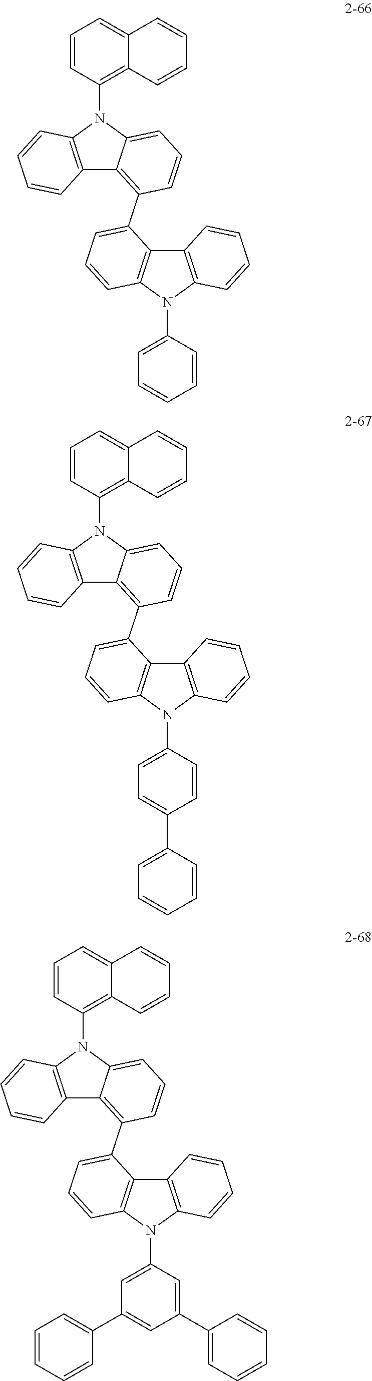

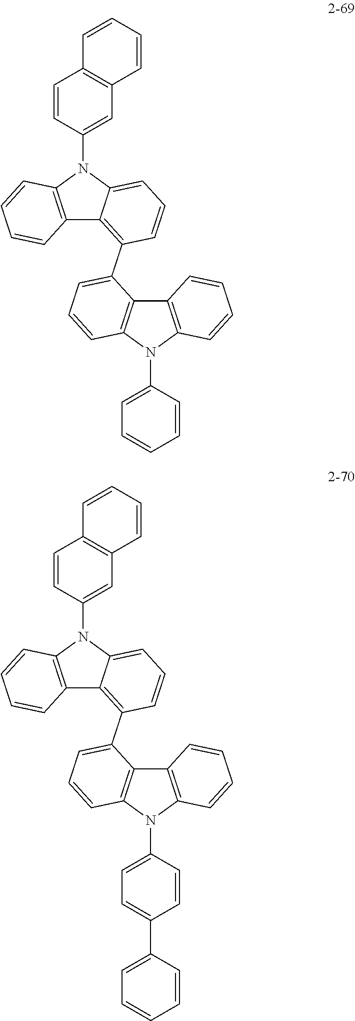

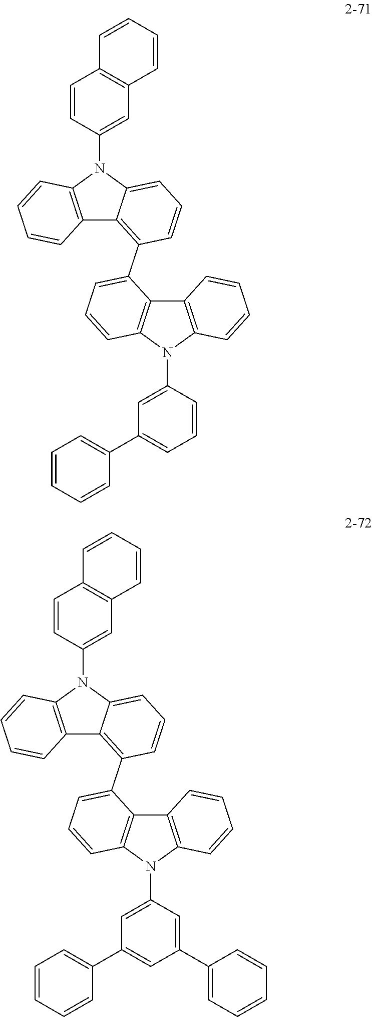

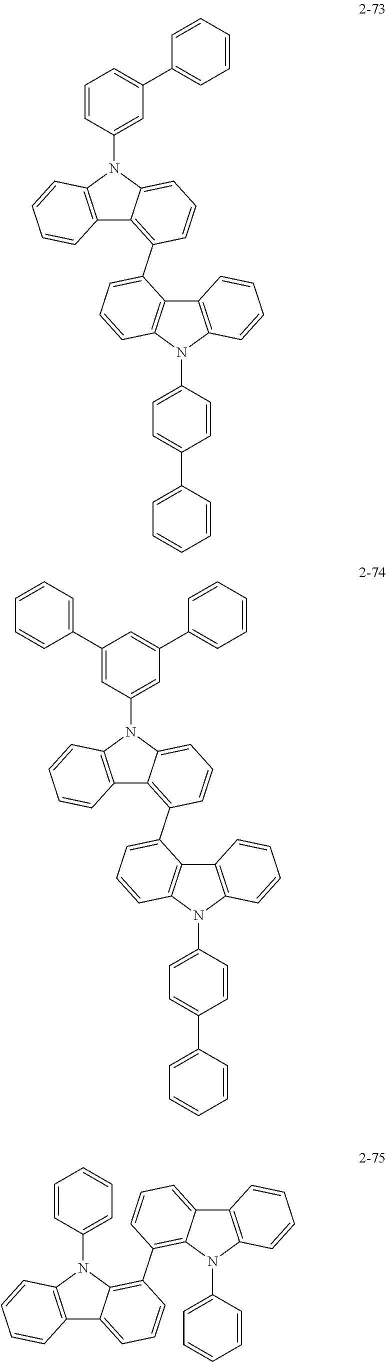

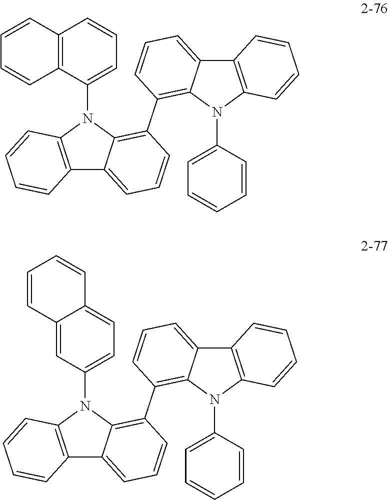

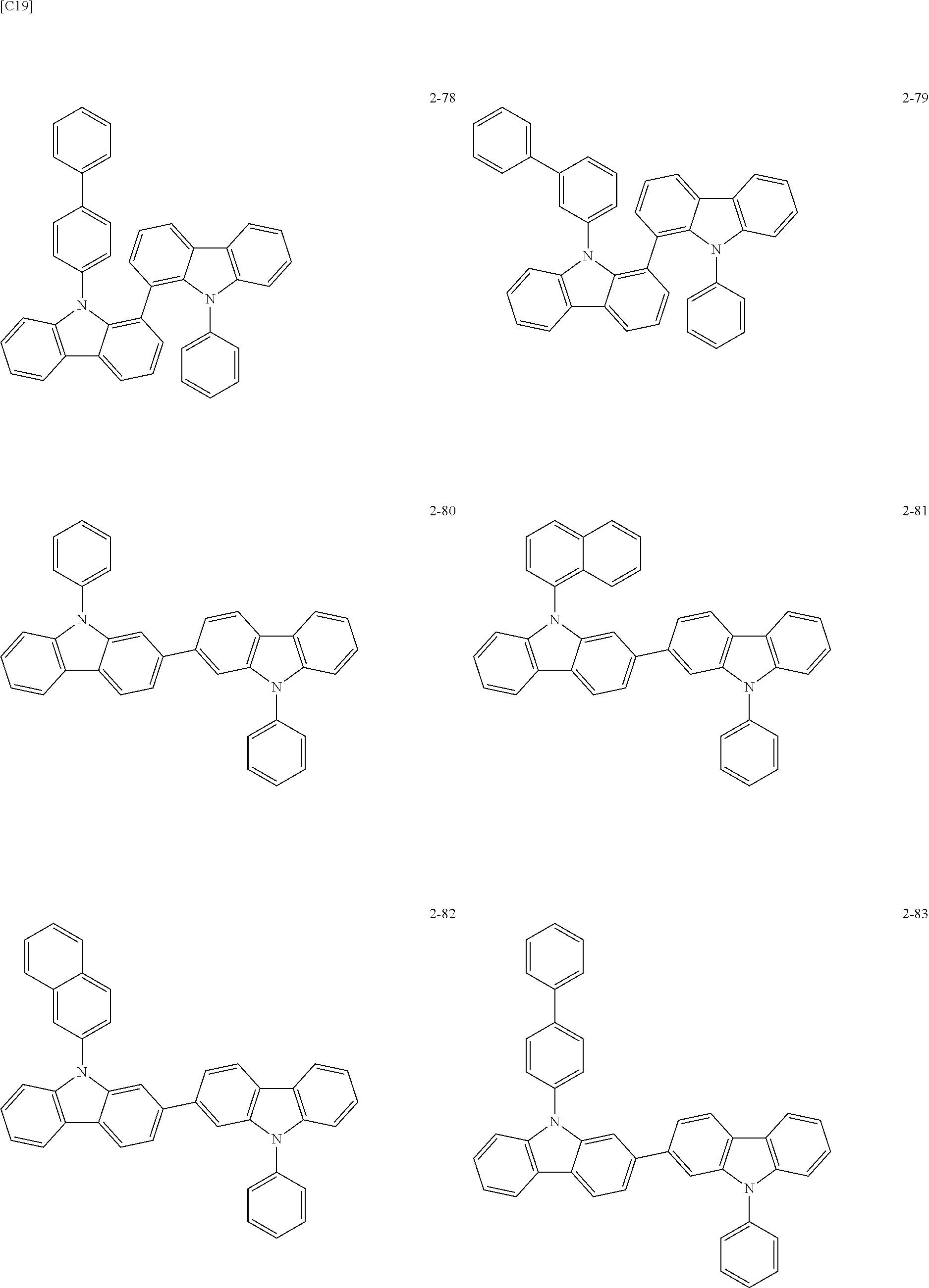

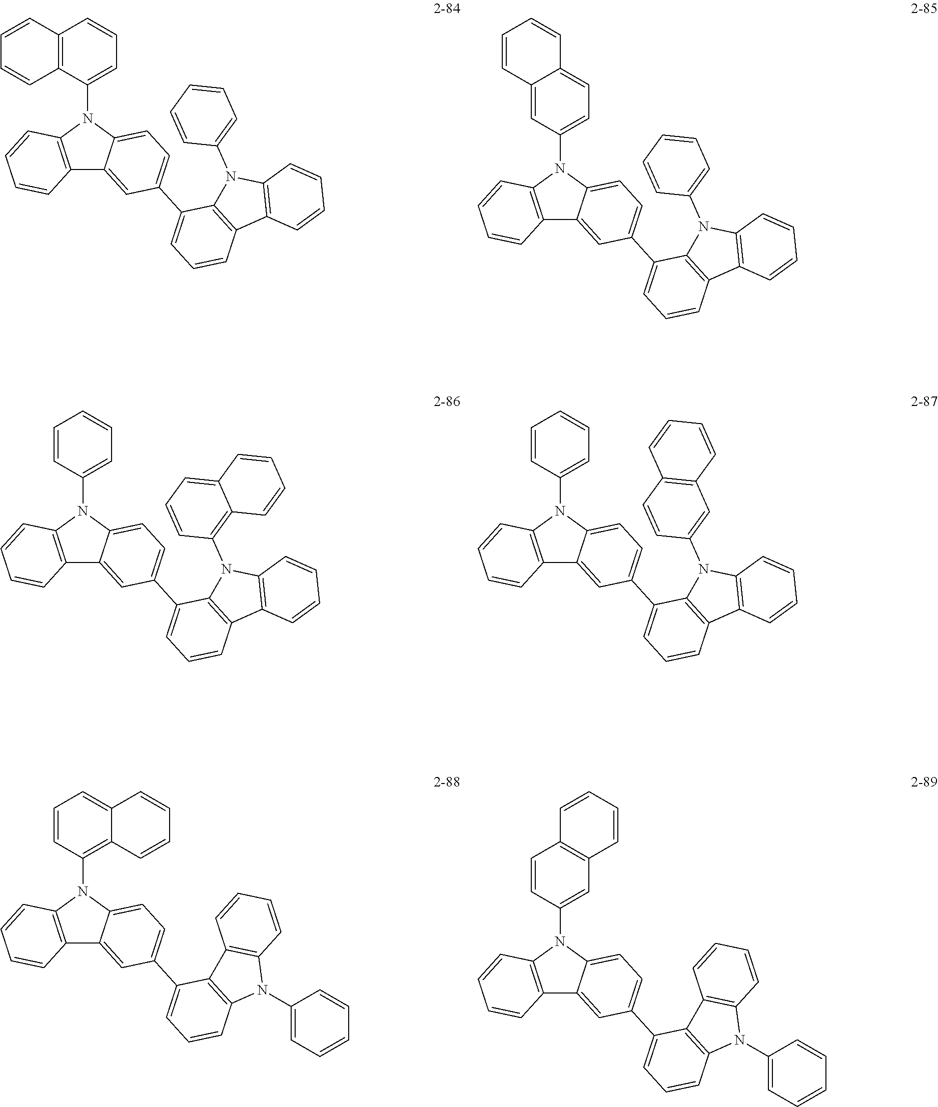

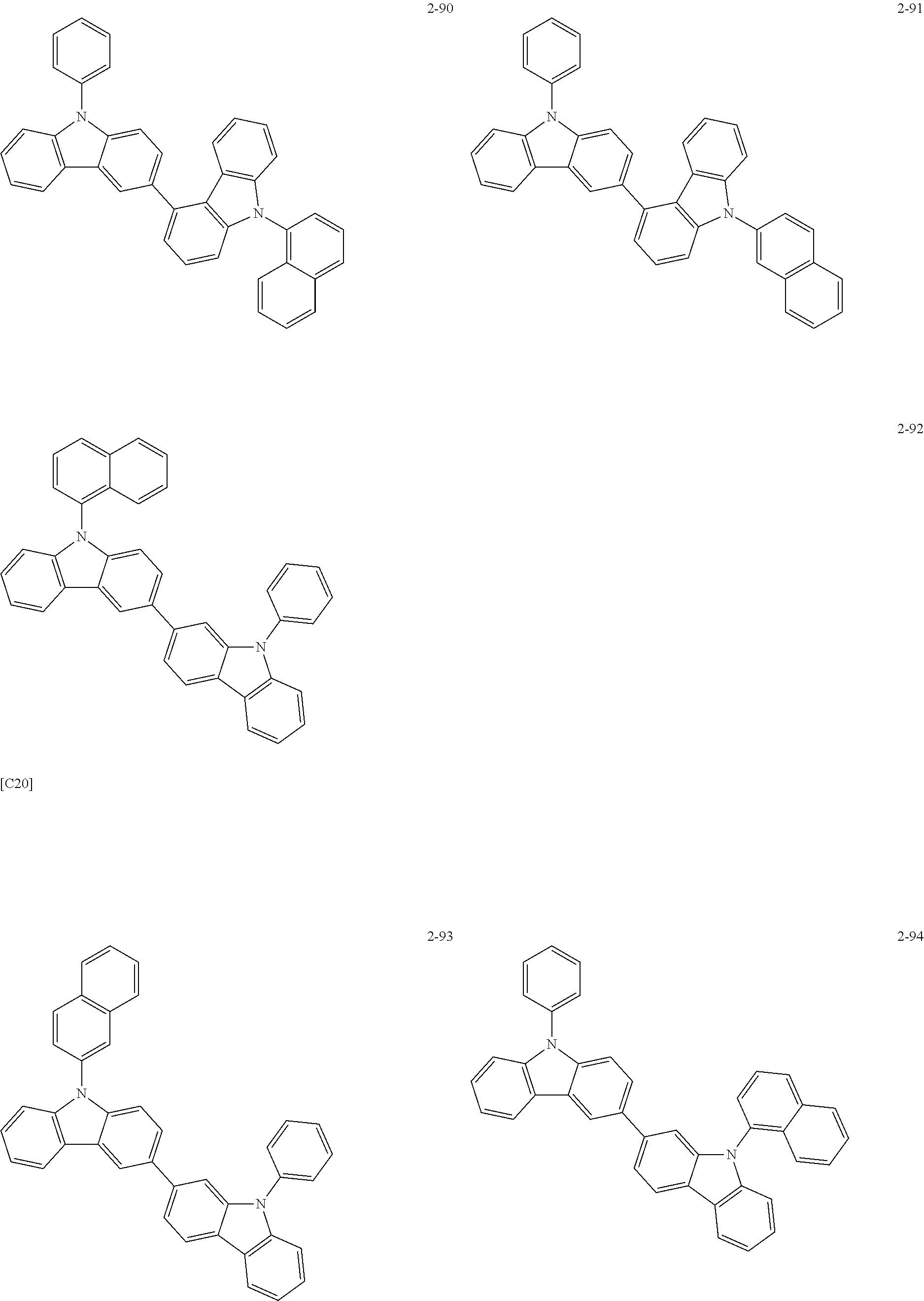

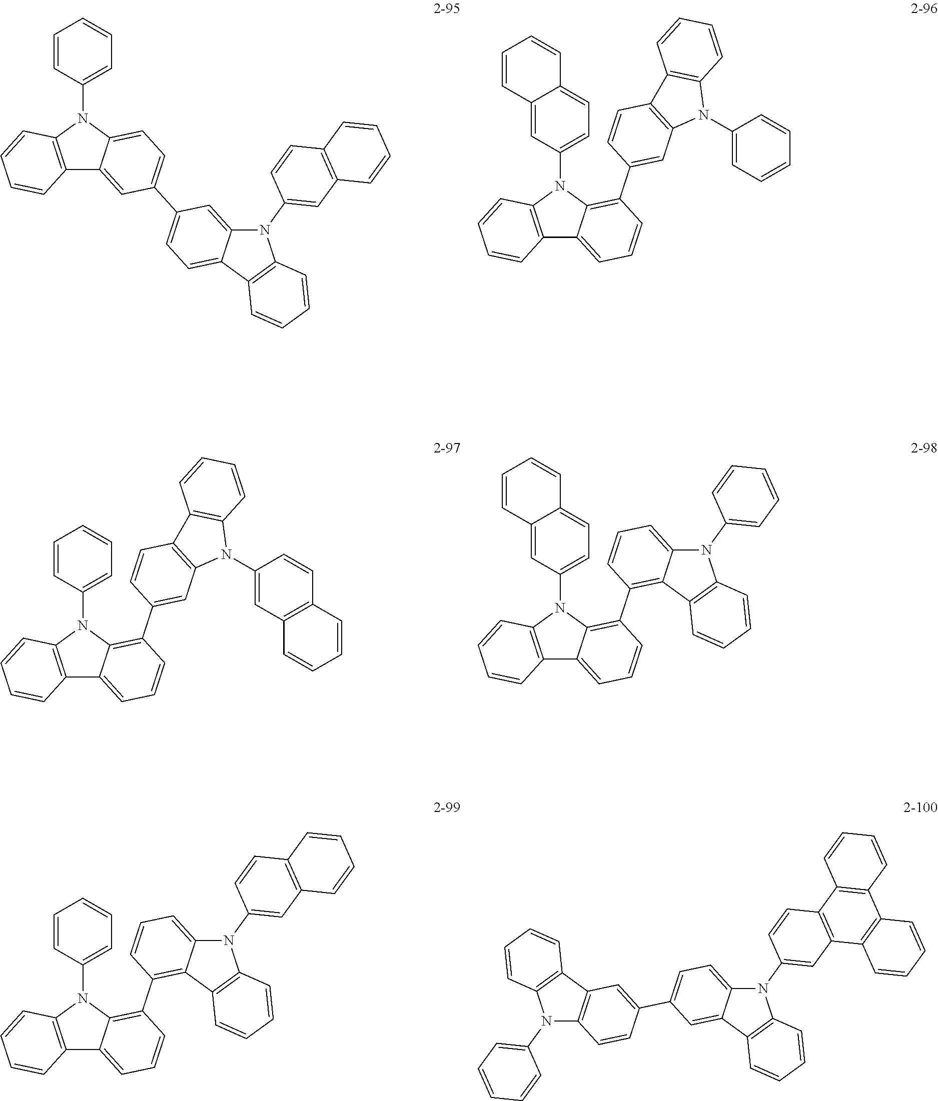

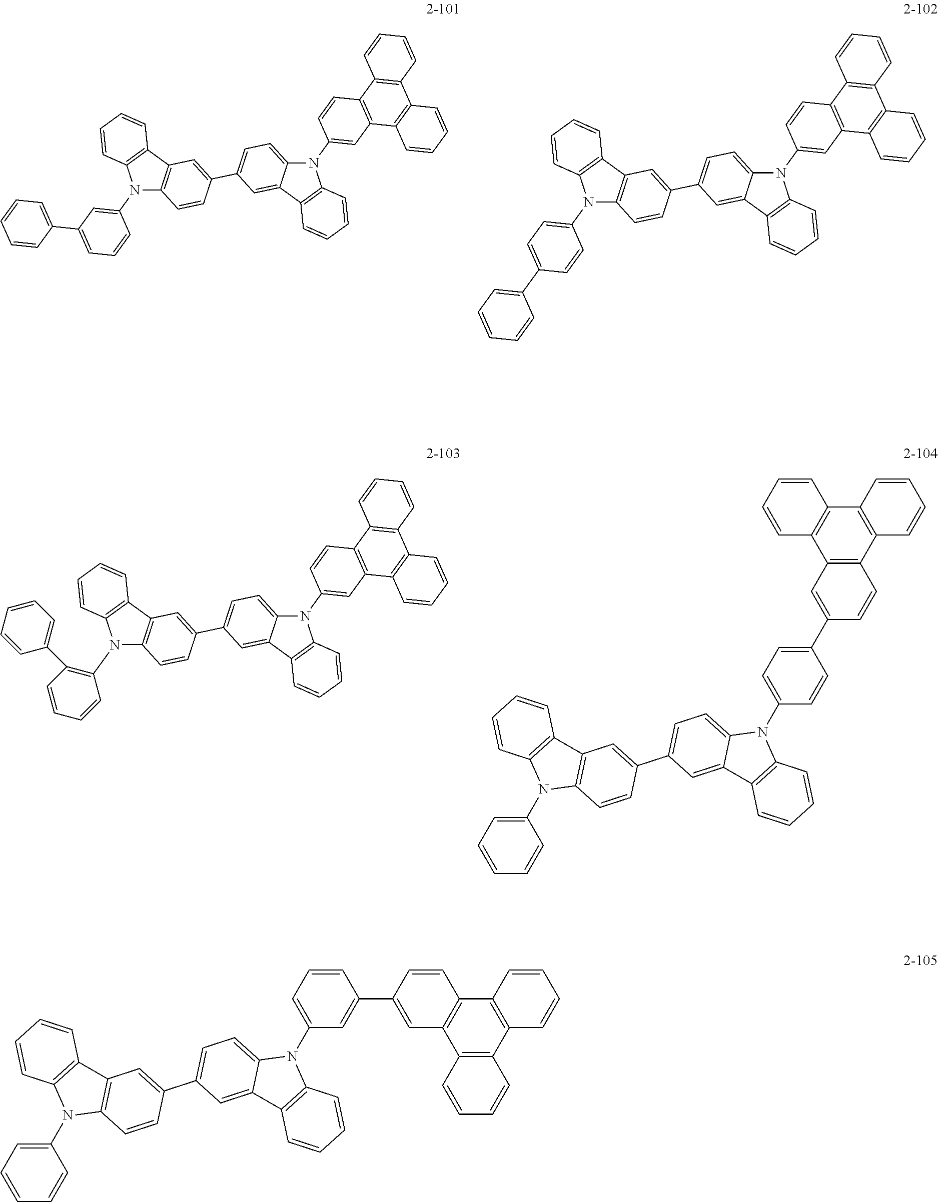

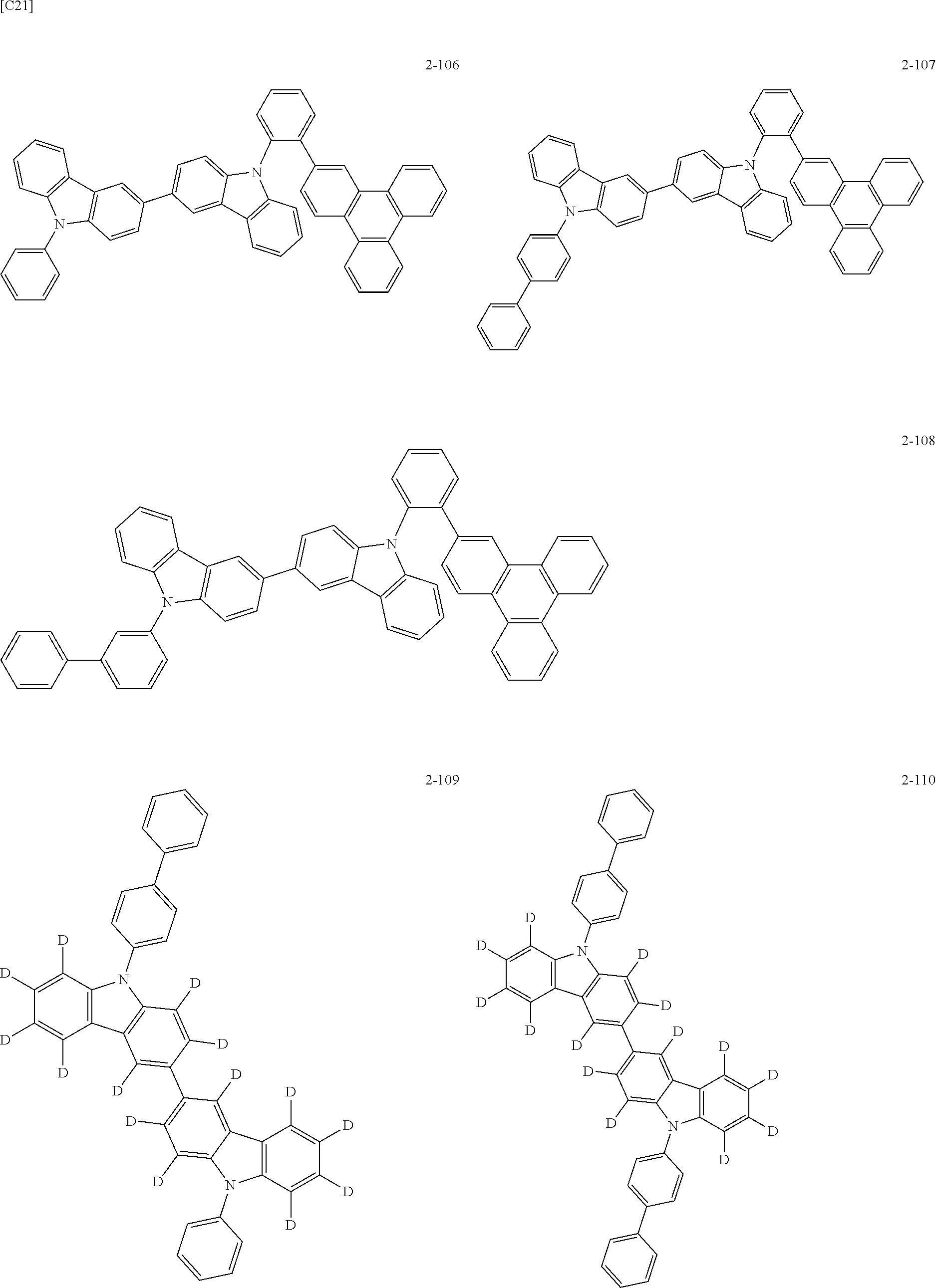

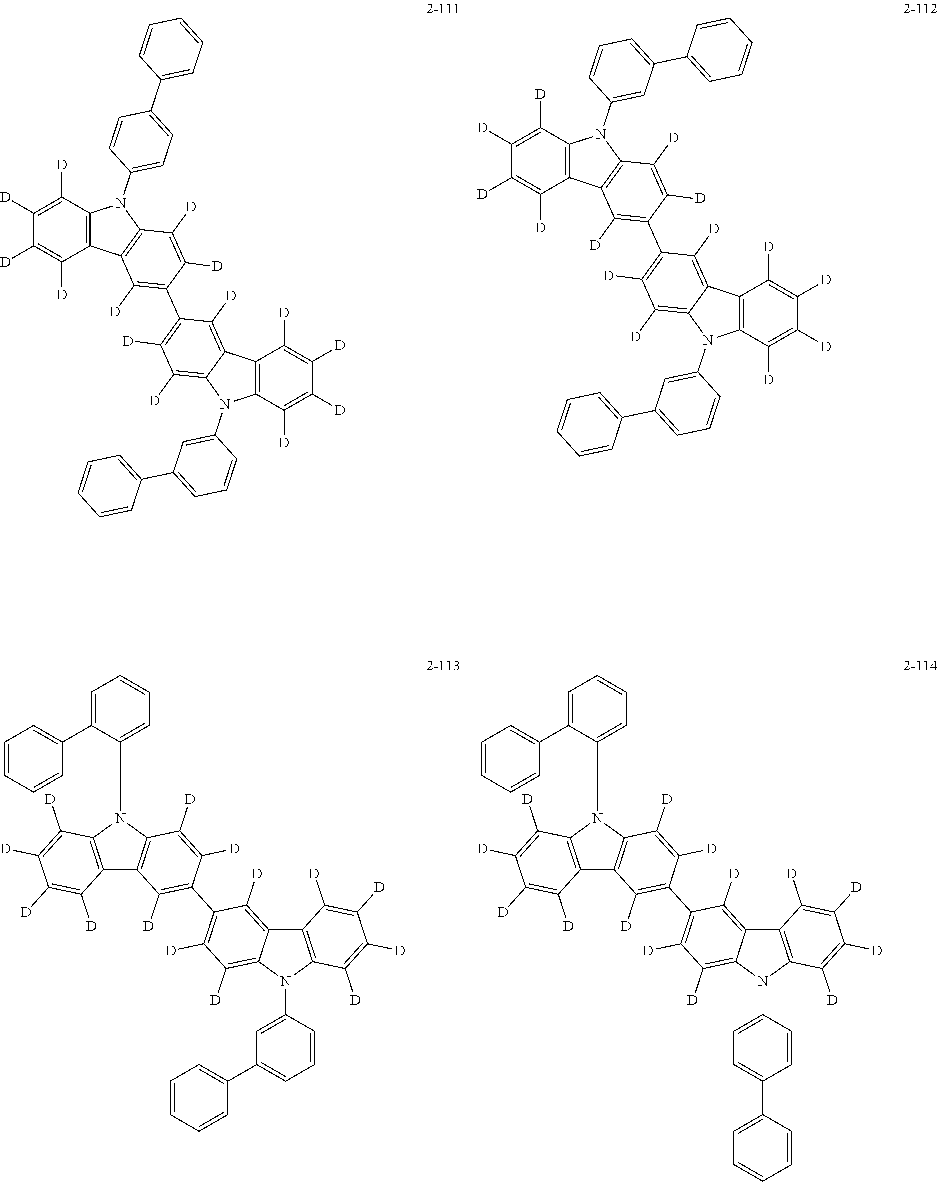

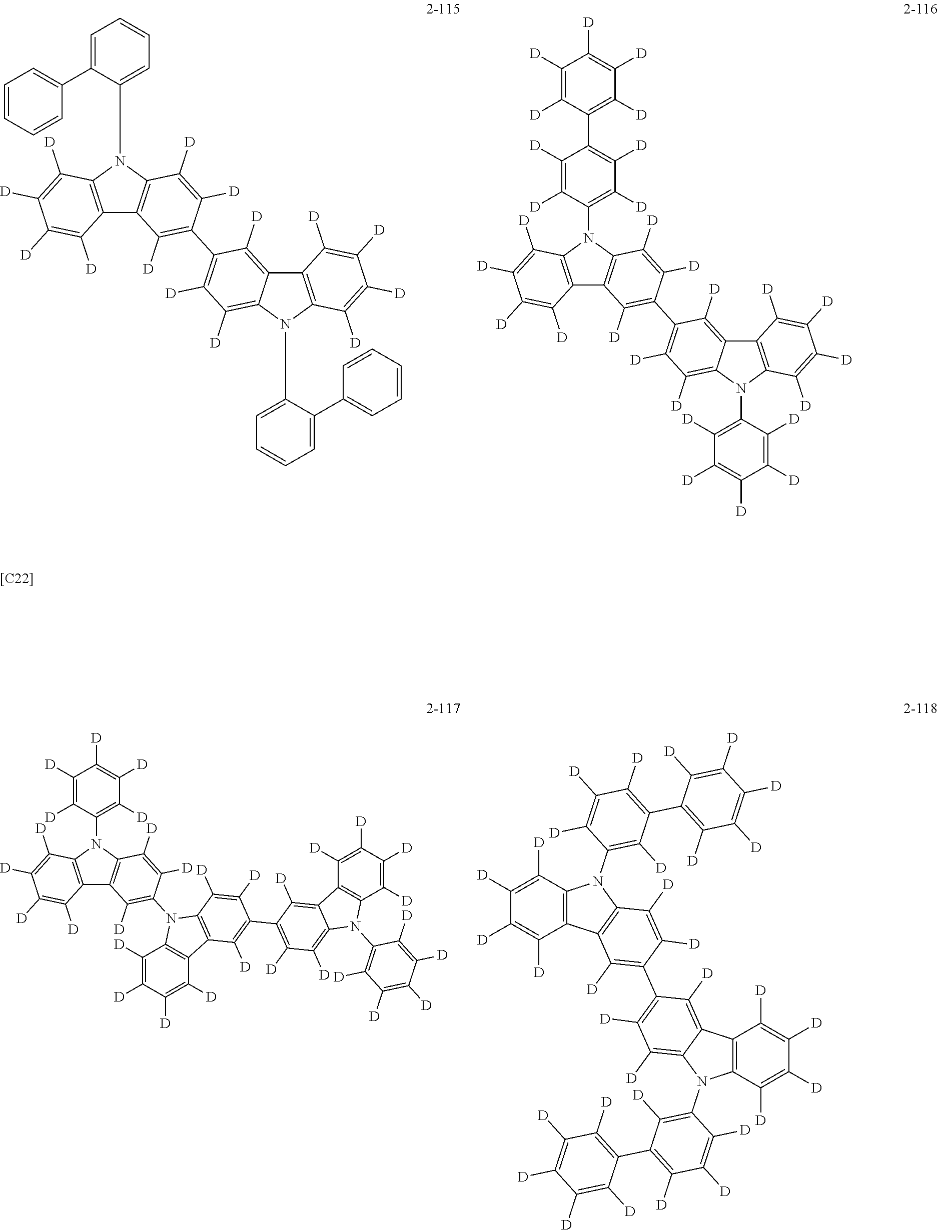

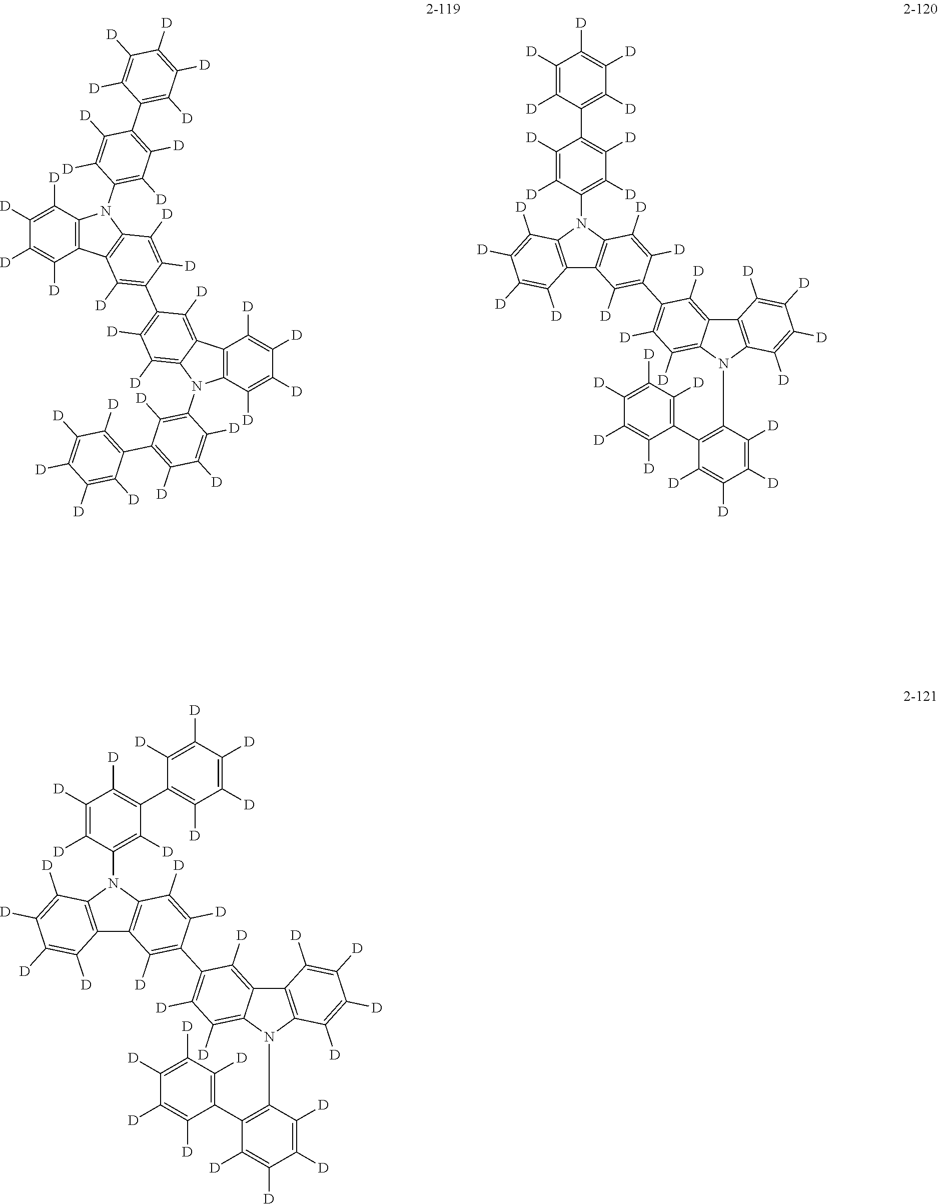

The host material for an organic EL device of the present invention is suitably used as a host material of a light-emitting layer.

Next, the structure of the organic EL device of the present invention will be described by referring to the drawing, but the structure of the organic EL device of the present invention is not limited thereto.

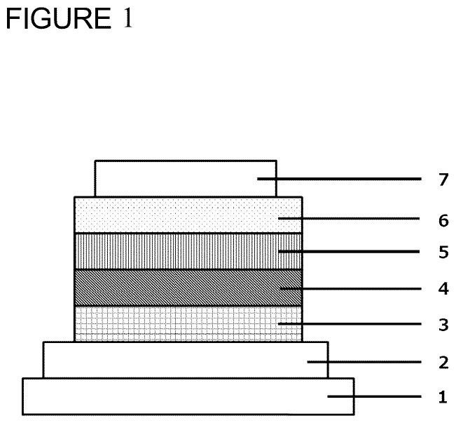

FIG. 1 is a cross-sectional view showing a structure example of an organic EL device generally used for the present invention, in which there are indicated a substrate 1, an anode 2, a hole injection layer 3, a hole transport layer 4, a light-emitting layer 5, an electron transport layer 6, and a cathode 7. The organic EL device of the present invention may have an exciton blocking layer adjacent to the light-emitting layer and may have an electron blocking layer between the light-emitting layer and the hole injection layer. The exciton blocking layer can be inserted into either of the anode side, and the cathode side of the light-emitting layer and inserted into both sides at the same time.

The organic EL device of the present invention has the anode, the light-emitting layer, and the cathode as essential layers, and preferably has a hole injection transport layer and an electron injection transport layer in addition to the essential layers, and further preferably has a hole blocking layer between the light-emitting layer and the electron injection transport layer. Note that the hole injection transport layer refers to either or both of a hole injection layer and a hole transport layer, and the electron injection transport layer refers to either or both of an electron injection layer and an electron transport layer.

A structure reverse to that of FIG. 1 is applicable, in which a cathode 7, an electron transport layer 6, a light-emitting layer 5, a hole transport layer 4, a hole injection layer 3, and an anode 2 are laminated on a substrate 1 in this order. In this case, layers may be added or omitted as necessary.

### —Substrate—

The organic EL device of the present invention is preferably supported on a substrate. The substrate is not particularly limited, and those conventionally used in organic EL devices may be used, and substrates made of, for example, glass, a transparent plastic, or quartz may be used.

### —Anode—

Regarding an anode material for an organic EL device, it is preferable to use a material of a metal, an alloy, an electrically conductive compound, and a mixture thereof, each having a large work function (4 eV or more). Specific examples of such an electrode material include a metal such as Au, and a conductive transparent material such as CuI, indium tin oxide (ITO), SnO₂, and ZnO. In addition, an amorphous material such as IDIXO (In₂O₃—ZnO), which is capable of forming a transparent conductive film, may be used. Regarding the anode, such an electrode material is used to form a thin film by, for example, a vapor-deposition or sputtering method, and a desired shape pattern may be formed by a photolithographic method; or if the pattern accuracy is not particularly required (about 100 μm or more), a pattern may be formed via a desired shape mask when the electrode material is vapor-deposited or sputtered. Alternatively, when a coatable substance such as an organic conductive compound is used, a wet film formation method such as a printing method or a coating method may be used. For taking emitted light from the anode, it is desired to have a transmittance of more than 10%, and the sheet resistance for the anode is preferably several hundreds Ω/or less. The film thickness is selected usually within 10 to 1000 nm, preferably within 10 to 200 nm though depending on the material.

### —Cathode—

Meanwhile, regarding a cathode material, a material of a metal (an electron injection metal), an alloy, an electrically conductive compound, or a mixture thereof, each having a small work function (4 eV or less) is used. Specific examples of such an electrode material include sodium, a sodium-potassium alloy, magnesium, lithium, a magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, and a rare earth metal. Among these, from the viewpoint of the electron injectability and the durability against oxidation and the like, a mixture of an electron injection metal and a second metal which is a stable metal having a larger work function value is suitable, and examples thereof include a magnesium/silver mixture, a magnesium/aluminum mixture, a magnesium/indium mixture, an aluminum/aluminum oxide mixture, a lithium/aluminum mixture and aluminum. The cathode can be produced by forming a thin film by a method such as vapor-depositing or sputtering of such a cathode material. In addition, the sheet resistance of cathode is preferably several hundreds Ω/or less. The film thickness is selected usually within 10 nm to 5 μm, preferably within 50 to 200 nm. Note that for transmission of emitted light, if either one of the anode and cathode of the organic EL device is transparent or translucent, emission luminance is improved, which is convenient.

In addition, formation of a film of the above metal with a thickness of 1 to 20 nm, on the cathode, followed by formation of a conductive transparent material described in the description on the anode thereon, enables production of a transparent or translucent cathode, and application of this enables production of a device wherein an anode and a cathode both have transmittance.

### —Light-Emitting Layer—

The light-emitting layer is a layer that emits light after excitons are generated when holes and electrons injected from the anode and the cathode, respectively, are recombined. As a light-emitting layer, an organic light-emitting dopant material and a host material may be contained.

As a host, a host material represented by any of the general formulas (1), (2) to (5) (also referred to as the host material of the present invention.) is used.

As the host material of the present invention, one kind thereof may be used, or two or more kinds of different compounds may be used, or one, or two or more other host materials such as known host materials may be used in combination. As other host material, a compound is preferable, which has hole transport ability and electron transport ability, which prevents elongation of the wavelength of light emitted, and which also has a high glass transition temperature.

When the host material of the present invention is contained as the first host material, the compound represented by any of the general formulas (6), (7) is particularly preferably used as the second host material, or other host material shown below may also be used as the second host material. When the host material of the present invention is used as the first host material and the compound represented by any of the general formulas (6), (7) is used as the second host material, other host material may also be used as a third host material.

Such other host material is one known in many Patent Literatures and the like, and can be selected therefrom. Specific examples of the host material include, but not particularly limited thereto, indolocarbazole derivatives described in WO2008/056746A1 or WO2008/146839A1, carbazole derivatives described in WO2009/086028A1 or WO2012/077520A1, CBP (N,N-biscarbazolylbiphenyl) derivatives, triazine derivatives described in WO2014/185595A1 or WO2018/021663A1, indenocarbazole derivatives described in WO2010/136109A1 or WO2011/000455A1, dibenzofuran derivatives described in WO2015/169412A1, triazole derivatives, indole derivatives, oxazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, styrylanthracene derivatives, fluorenone derivatives, hydrazone derivatives, stilbene derivatives, silazane derivatives, aromatic tert-amine compounds, styrylamine compounds, aromatic dimethylidene-based compounds, porphyrin-based compounds, anthraquinodimethane derivatives, anthrone derivatives, diphenylquinone derivatives, thiopyran dioxide derivatives, heterocyclic tetracarboxylic anhydrides of naphthalene, perylene, or the like, various metal complexes typified by phthalocyanine derivatives, metal complexes of 8-quinolinol derivatives, metal phthalocyanine, and metal complexes of benzoxazole or benzothiazole derivatives, and polymer compounds such as polysilane-based compounds, poly(N-vinylcarbazole) derivatives, aniline-based copolymers, thiophene oligomers, polythiophene derivatives, polyphenylene derivatives, polyphenylene vinylene derivatives, and polyfluorene derivatives.

Specific examples of other host material described above are shown below, but are not limited thereto.

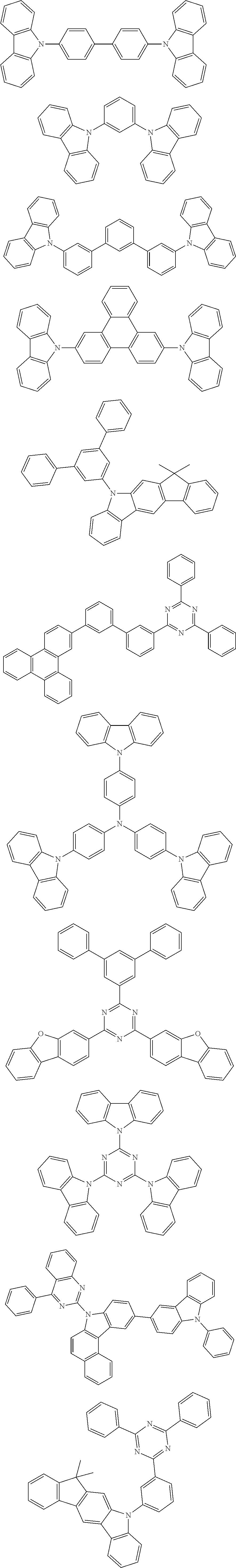

Preferred examples of the organic light-emitting dopant material include a phosphorescent dopant, a fluorescence-emitting dopant or a thermally activated delayed fluorescence-emitting dopant.

Preferred is a phosphorescent dopant including an organic metal complex containing at least one metal selected from ruthenium, rhodium, palladium, silver, rhenium, osmium, iridium, platinum and gold. Specifically, iridium complexes described in J. Am. Chem. Soc. 2001, 123, 4304, JP2013-530515A, US2016/0049599A1, US2017/0069848A1, US2018/0282356A1, US2019/0036043A1, and the like, or platinum complexes described in US2018/0013078A1, KR2018/094482A, and the like are preferably used, but the phosphorescent dopant material is not limited thereto.

Regarding the phosphorescent dopant material, only one kind thereof may be contained in the light-emitting layer, or two or more kinds thereof may be contained. A content of the phosphorescent dopant material is preferably 0.1 to 30 mass % and more preferably 1 to 20 mass % with respect to the host material.

The phosphorescent dopant material is not particularly limited, and specific examples thereof include the following.

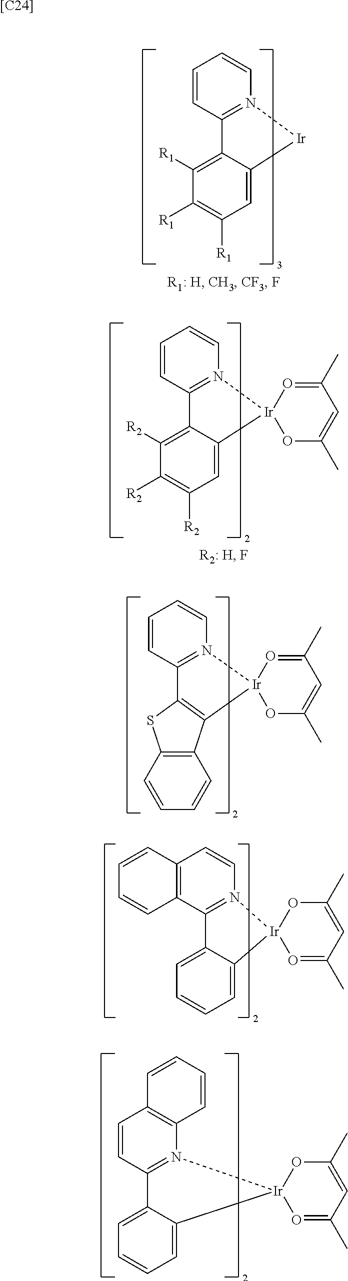

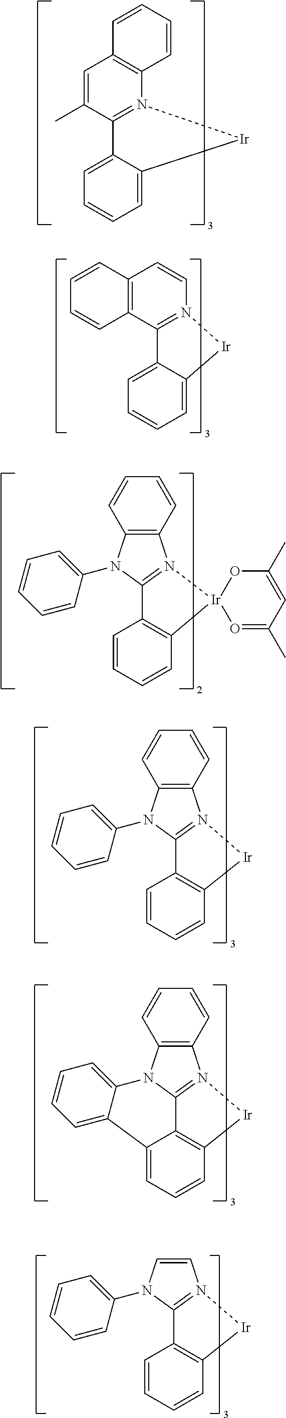

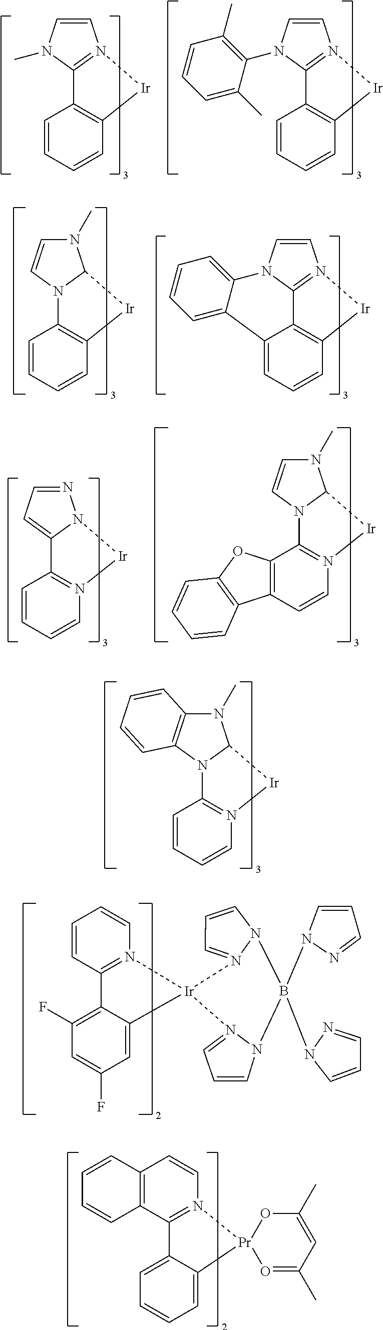

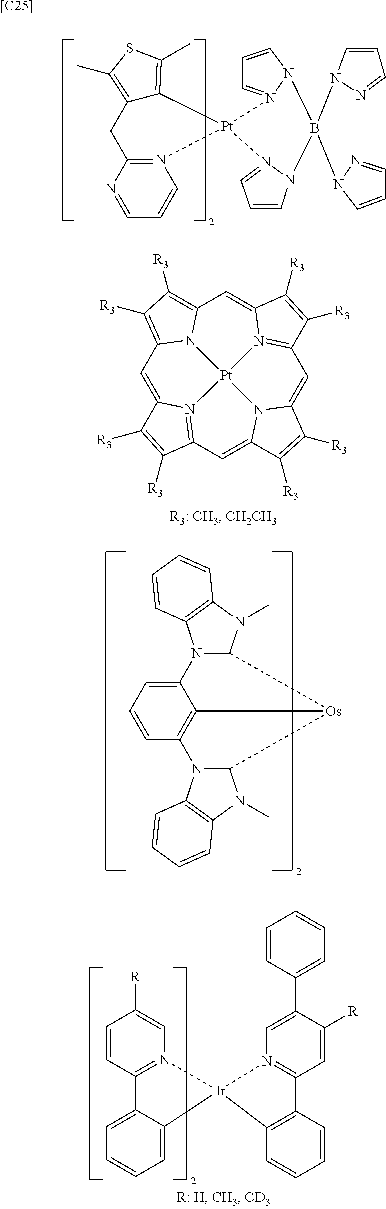

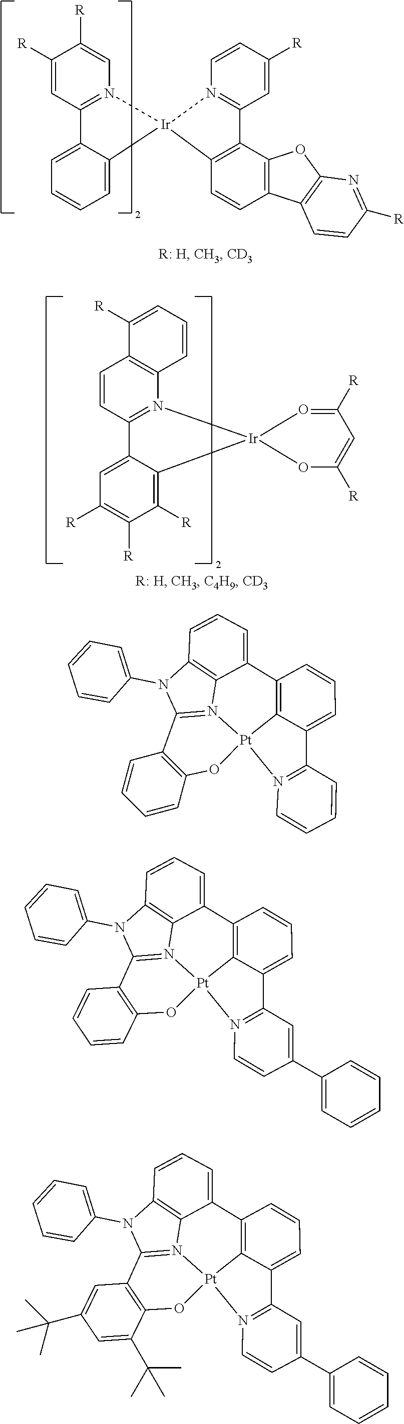

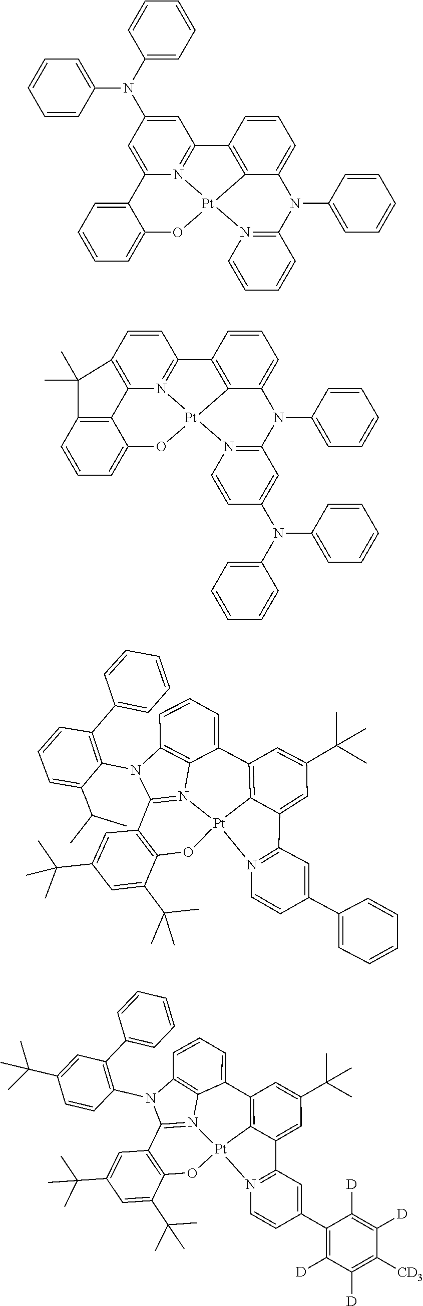

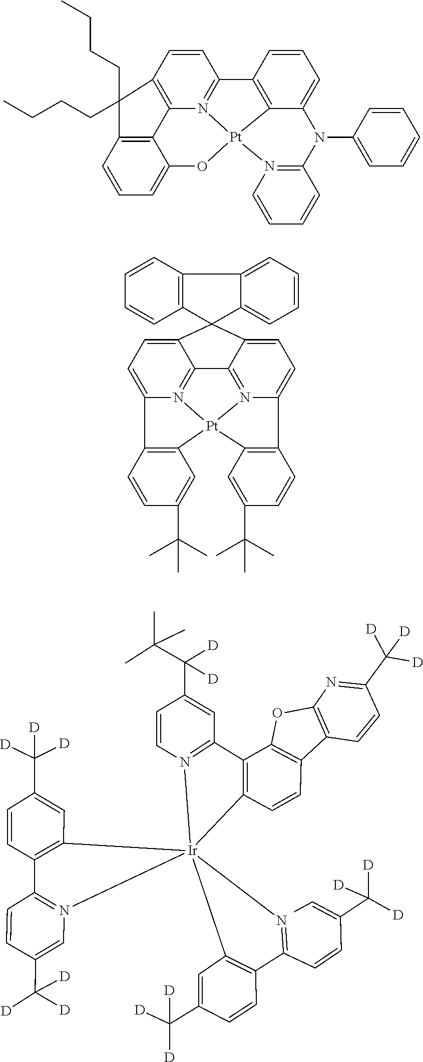

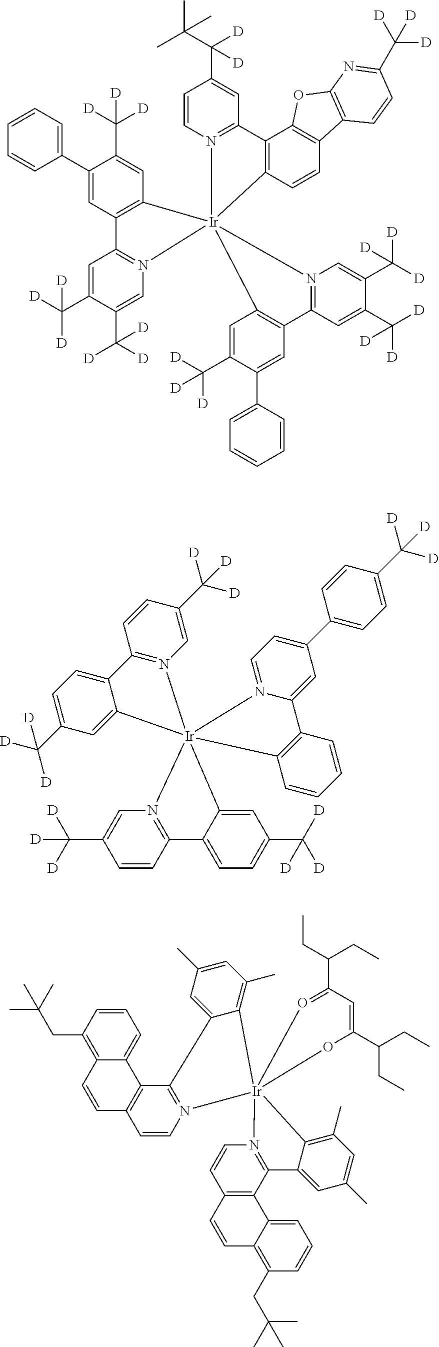

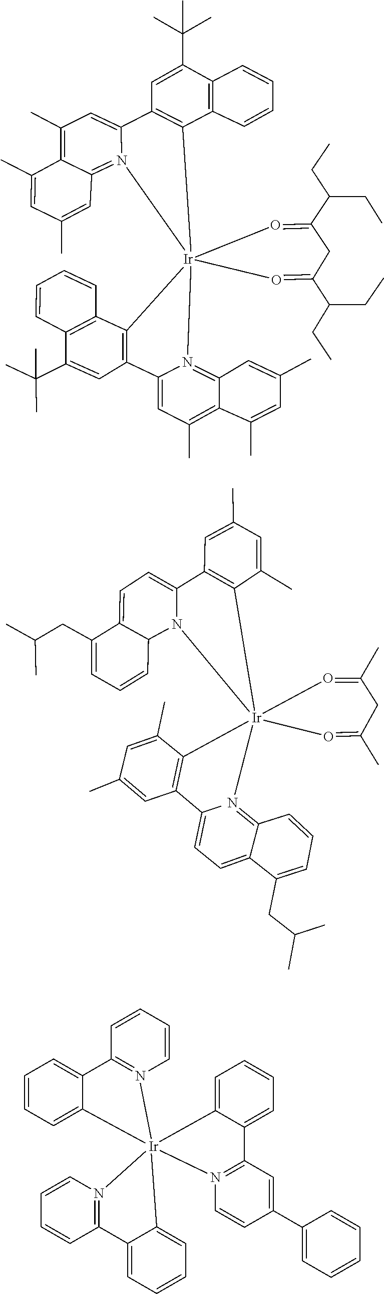

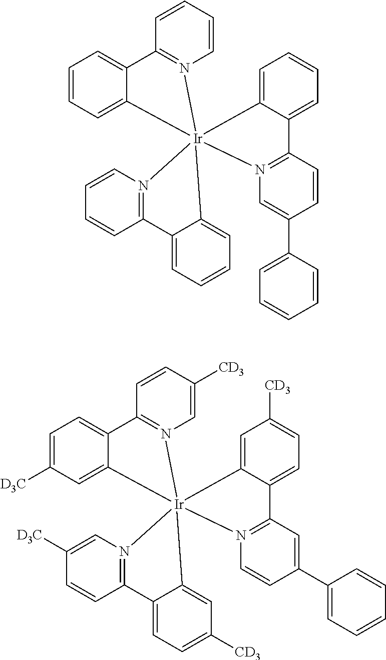

The fluorescence-emitting dopant is not particularly limited. Examples thereof include benzoxazole derivatives, benzothiazole derivatives, benzimidazole derivatives, styrylbenzene derivatives, polyphenyl derivatives, diphenylbutadiene derivatives, tetraphenyl butadiene derivatives, naphthalimido derivatives, coumarin derivatives, fused aromatic compounds, perinone derivatives, oxadiazole derivatives, oxazine derivatives, aldazine derivatives, pyrrolidine derivatives, cyclopentadiene derivatives, bisstyryl anthracene derivatives, quinacridone derivatives, pyrrolopyridine derivatives, thiadiazolopyridine derivatives, styrylamine derivatives, diketopyrrolopyrrole derivatives, aromatic dimethylidine compounds, metal complexes of 8-quinolinol derivatives or metal complexes of pyromethene derivatives, rare earth complexes, various metal complexes represented by transition metal complexes, polymer compounds such as polythiophene, polyphenylene, and polyphenylene vinylene, and organosilane derivatives. Preferred examples thereof include fused aromatic derivatives, styryl derivatives, diketopyrrolopyrrole derivatives, oxazine derivatives, pyromethene metal complexes, transition metal complexes, and lanthanoid complexes. More preferable examples thereof include naphthalene, pyrene, chrysene, triphenylene, benzo[c]phenanthrene, benzo[a]anthracene, pentacene, perylene, fluoranthene, acenaphthofluoranthene, dibenzo[a,j]anthracene, dibenzo[a,h]anthracene, benzo[a]naphthalene, hexacene, naphtho[2,1-f]isoquinoline, α-naphthaphenanthridine, phenanthrooxazole, quinolino[6,5-f]quinoline, and benzothiophanthrene. These may have an alkyl group, an aryl group, an aromatic heterocyclic group, or a diarylamino group as a substituent.

Regarding the fluorescence-emitting dopant material, only one kind thereof may be contained in the light-emitting layer, or two or more kinds thereof may be contained. A content of the fluorescence-emitting dopant material is preferably 0.1 to 20 mass % and more preferably 1 to 10 mass % with respect to the host material.

The thermally activated delayed fluorescence-emitting dopant is not particularly limited. Examples thereof include: metal complexes such as a tin complex and a copper complex; indolocarbazole derivatives described in WO2011/070963A1; cyanobenzene derivatives and carbazole derivatives described in Nature 2012, 492, 234; and phenazine derivatives, oxadiazole derivatives, triazole derivatives, sulfone derivatives, phenoxazine derivatives, and acridine derivatives described in Nature Photonics 2014, 8,326.

The thermally activated delayed fluorescence-emitting dopant material is not particularly limited, and specific examples thereof include the following.

### [C27]

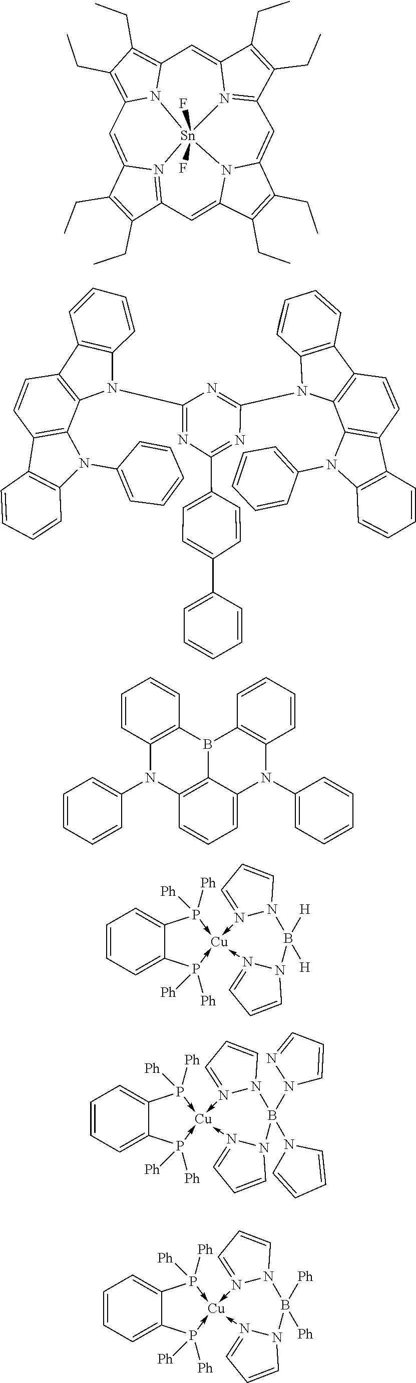

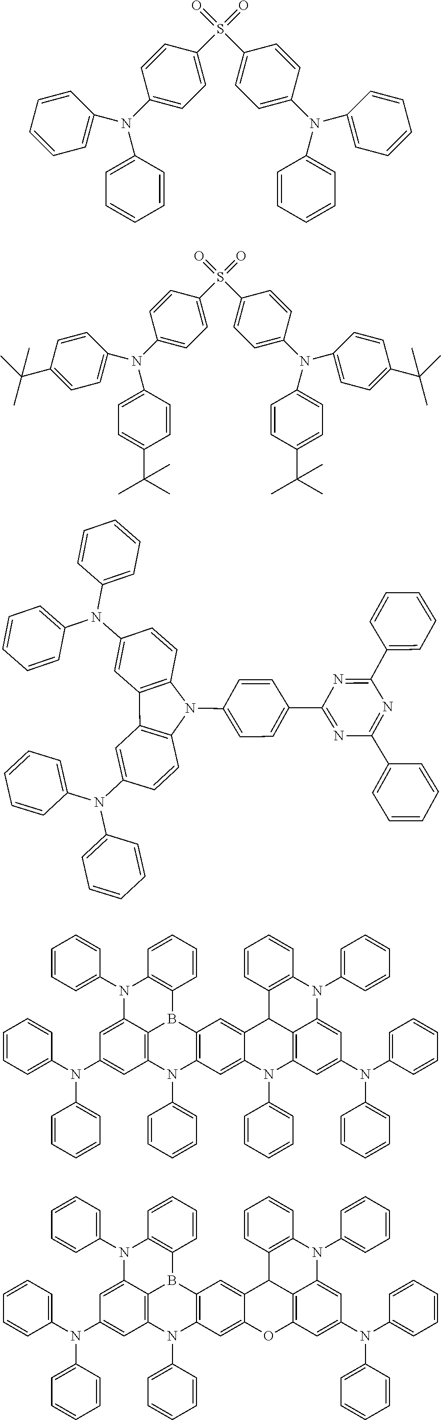

Regarding the thermally activated delayed fluorescence-emitting dopant material, only one kind thereof may be contained in the light-emitting layer, or two or more kinds thereof may be contained. In addition, the thermally activated delayed fluorescence-emitting dopant may be used by mixing with a phosphorescent dopant and a fluorescence-emitting dopant. A content of the thermally activated delayed fluorescence-emitting dopant material is preferably 0.1% to 50 mass % and more preferably 1% to 30 mass % with respect to the host material.

### —Injection Layer—

The injection layer is a layer that is provided between an electrode and an organic layer in order to lower a driving voltage and improve emission luminance, and includes a hole injection layer and an electron injection layer, and may be present between the anode and the light-emitting layer or the hole transport layer, and between the cathode and the light-emitting layer or the electron transport layer. The injection layer can be provided as necessary.

### —Hole Blocking Layer—

The hole blocking layer has a function of the electron transport layer in a broad sense, and is made of a hole blocking material having a function of transporting electrons and a significantly low ability to transport holes, and can block holes while transporting electrons, thereby improving a probability of recombining electrons and holes in the light-emitting layer.

### —Electron Blocking Layer—

The electron blocking layer has a function of a hole transport layer in a broad sense and blocks electrons while transporting holes, thereby enabling a probability of recombining electrons and holes in the light-emitting layer to be improved.

Regarding the material of the electron blocking layer, a known electron blocking layer material can be used and a material of the hole transport layer to be described below can be used as necessary. A film thickness of the electron blocking layer is preferably 3 to 100 nm, and more preferably 5 to 30 nm.

### —Exciton Blocking Layer—

The exciton blocking layer is a layer for preventing excitons generated by recombination of holes and electrons in the light-emitting layer from being diffused in a charge transport layer, and insertion of this layer allows excitons to be efficiently confined in the light-emitting layer, enabling the luminous efficiency of the device to be improved. The exciton blocking layer can be inserted, in a device having two or more light-emitting layers adjacent to each other, between two adjacent light-emitting layers.

Regarding the material of the exciton blocking layer, a known exciton blocking layer material can be used. Examples thereof include 1,3-dicarbazolyl benzene (mCP) and bis(2-methyl-8-quinolinolato)-4-phenylphenolato aluminum (III) (BAlq).

### —Hole Transport Layer—

The hole transport layer is made of a hole transport material having a function of transporting holes, and the hole transport layer can be provided as a single layer or a plurality of layers.

The hole transport material has any of hole injection properties, hole transport properties, or electron barrier properties, and may be either an organic material or an inorganic material. For the hole transport layer, any one selected from conventionally known compounds can be used. Examples of such a hole transport material include porphyrin derivatives, arylamine derivatives, triazole derivatives, oxadiazole derivatives, imidazole derivatives, polyarylalkane derivatives, pyrazoline derivatives and pyrazolone derivatives, phenylenediamine derivatives, arylamine derivatives, amino-substituted chalcone derivatives, oxazole derivatives, styryl anthracene derivatives, fluorenone derivatives, hydrazone derivatives, stilbene derivatives, silazane derivatives, an aniline copolymer, and a conductive polymer oligomer, and particularly a thiophene oligomer. Use of porphyrin derivatives, arylamine derivatives, or styrylamine derivatives is preferred. Use of arylamine derivatives is more preferred.

### —Electron Transport Layer—

The electron transport layer is made of a material having a function of transporting electrons, and the electron transport layer can be provided as a single layer or a plurality of layers.

The electron transport material (which may also serve as a hole blocking material) may have a function of transferring electrons injected from the cathode to the light-emitting layer. For the electron transport layer, any one selected from conventionally known compounds can be used, and examples thereof include polycyclic aromatic derivatives such as naphthalene, anthracene, and phenanthroline, tris(8-quinolinolato)aluminum(III) derivatives, phosphine oxide derivatives, nitro-substituted fluorene derivatives, diphenylquinone derivatives, thiopyran dioxide derivatives, carbodiimide, fluorenylidene methane derivatives, anthraquinodimethane derivatives and anthrone derivatives, bipyridine derivatives, quinoline derivatives, oxadiazole derivatives, benzimidazole derivatives, benzothiazole derivatives, and indolocarbazole derivatives. In addition, a polymer material in which the above material is introduced into a polymer chain or the above material is used for a main chain of a polymer can be used.

The method for producing the organic EL device of the present invention comprises a step of mixing the first host material and the second host material in advance, and a step of vapor-depositing the resulting mixture from one vapor deposition source to form a light-emitting layer. Thus, two host materials can be mixed in advance, resulting in an enhancement in performance of an organic EL device. The mixing method here adopted can be powder mixing, melting and mixing, or sublimation. The form of the host and the premixture thereof may be powder, sticks, or granules.

The difference in temperature at 50% weight loss of the first host material and the second host material in the composition obtained by the above mixing in advance (also referred to as the premixture) is preferably within 20° C.

The 50% weight reduction temperature is a temperature at which the weight is reduced by 50% when the temperature is raised from room temperature to 550° C. at a rate of 10° C./min in TG-DTA measurement under a nitrogen stream reduced pressure (1 Pa). It is considered that vaporization due to evaporation or sublimation the most vigorously occurs around this temperature.

### EXAMPLES

Hereafter, the present invention will be described in more detail by referring to Examples, but the present invention is not limited to these Examples and can be implemented in various forms without departing from the gist thereof.

Synthesis Examples of compound 1-11, compound 2-109, and compound 2-116 are shown as representative examples. Other compounds were also synthesized by similar methods. Rates of deuteration were determined by proton nuclear magnetic resonance spectroscopy.

### Synthesis Example 1

### [C28]

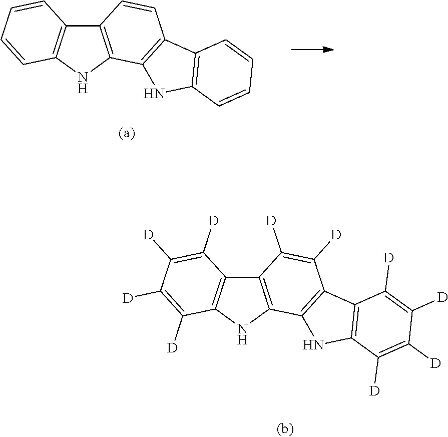 [SMILES: c1ccc2c(c1)[nH]c1c2ccc2c3ccccc3[nH]c21 ; [2H]c1c([2H])c([2H])c2c([nH]c3c4[nH]c5c([2H])c([2H])c([2H])c([2H])c5c4c([2H])c([2H])c32)c1[2H]]

To 15.0 g (0.0585 mol) of compound (a) were added 148 ml of deuterated benzene (C₆D₆) and 43.9 g (0.293 mol) of deuterated trifluoromethanesulfonic acid (TfOD), and the mixture was heated and stirred under a nitrogen atmosphere at 30° C. for 2 hours. The reaction liquid was added to a solution of sodium carbonate (34.1 g) in deuterated water (341 mL) in an ice bath, and the mixture was stirred for 1 hour. Further, 300 mL of toluene was added to extract an organic layer, and the organic layer was dried over MgSO₄, MgSO₄ was filtered off, and the filtrate was concentrated. 300 mL of MeOH was added to the concentrate, the mixture was stirred at room temperature for 2 hours, and the precipitated crystal was collected by filtration to give 14.9 g (0.0555 mol, yield 94.9%, rate of deuteration 91%) of compound (b) as a deuterated product. (APCI-TOFMS, m/z 267 [M+H]⁺).

### Synthesis Example 2

### [C29]

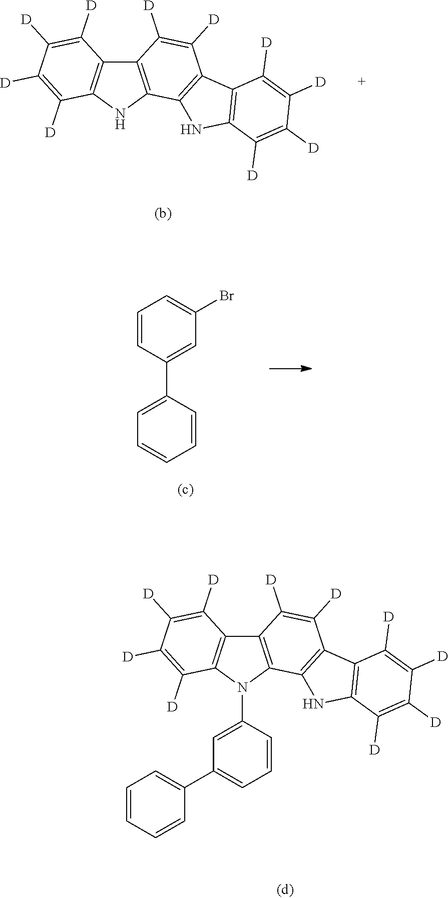 [SMILES: [2H]c1c([2H])c([2H])c2c([nH]c3c4[nH]c5c([2H])c([2H])c([2H])c([2H])c5c4c([2H])c([2H])c32)c1[2H] ; Brc1cccc(-c2ccccc2)c1 ; [2H]c1c([2H])c([2H])c2c([nH]c3c2c([2H])c([2H])c2c4c([2H])c([2H])c([2H])c([2H])c4n(-c4cccc(-c5ccccc5)c4)c32)c1[2H]]

To 5.00 g (0.0186 mol) of compound (b) were added 4.78 g (0.0205 mol) of compound (c), 0.35 g (0.00186 mol) of copper iodide, 5.15 g (0.0370 mol) of potassium carbonate, 0.54 g (0.00373 mol) of 8-quinolinol, and 21.5 g of 1,3-dimethyl-2-imidazolidinone, and the mixture was stirred under a nitrogen atmosphere at 130° C. for 3 hours. The mixture was cooled to room temperature, and then added dropwise to a mixed solution of 41 g of water and 10 g of 2NHCl, and the mixture was stirred at room temperature for 2 hours. The precipitated crystal was collected by filtration and purified by silica gel column chromatography (toluene). The resulting crystal was purified by crystallization using 12 g of acetone and 12 g of heptane to give 3.70 g (0.00882 mol, yield 47.4%, rate of deuteration 50%) of compound (d) as a white solid. (APCI-TOFMS, m/z 419 [M+H]⁺).

### Synthesis Example 3

### [C30]

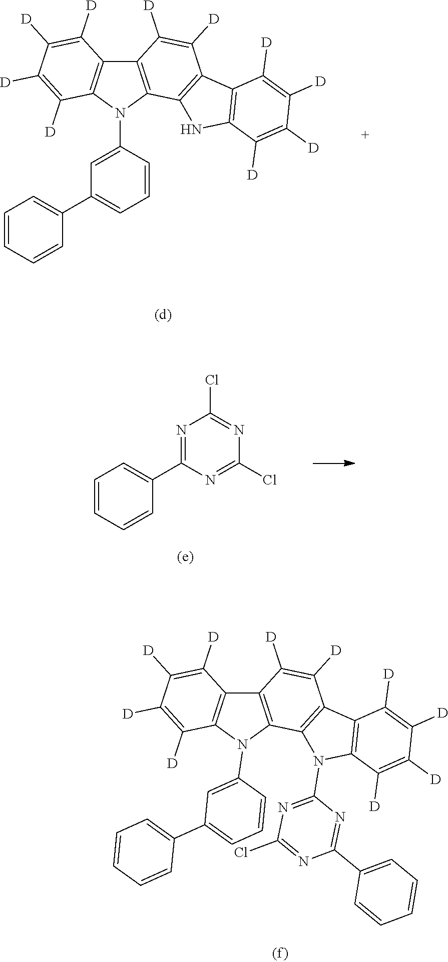 [SMILES: [2H]c1c([2H])c([2H])c2c([nH]c3c2c([2H])c([2H])c2c4c([2H])c([2H])c([2H])c([2H])c4n(-c4cccc(-c5ccccc5)c4)c32)c1[2H] ; Clc1nc(Cl)nc(-c2ccccc2)n1 ; [2H]c1c([2H])c([2H])c2c(c1[2H])c1c([2H])c([2H])c3c4c([2H])c([2H])c([2H])c([2H])c4n(-c4nc(Cl)nc(-c5ccccc5)n4)c3c1n2-c1cccc(-c2ccccc2)c1]

Under a nitrogen atmosphere, 0.27 g (0.00694 mol) of 62.6 wt % sodium hydride was added to 25 g of N,N′-dimethylacetamide to prepare a suspension. To this was added 2.76 g (0.00658 mol) of compound (d) dissolved in 5 g of N,N′-dimethylacetamide, and the mixture was stirred at 15° C. for 30 minutes. After the reaction solution was cooled to 5° C., 1.65 g (0.00708 mol) of compound (e) was added, and then the mixture was stirred for 2 hours while being maintained at 5° C. To the reaction solution was added distilled water (18 mL) and stirred for 1 hour, and the precipitated solid was collected by filtration. The resulting solid was purified by crystallization using 28 g of acetonitrile to give 3.62 g (0.00595 mol, yield 90.4%, rate of deuteration 32%) of compound (f) as a yellow solid. (APCI-TOFMS, m/z 608 [M+H]⁺).

### Synthesis Example 4

### [C31]

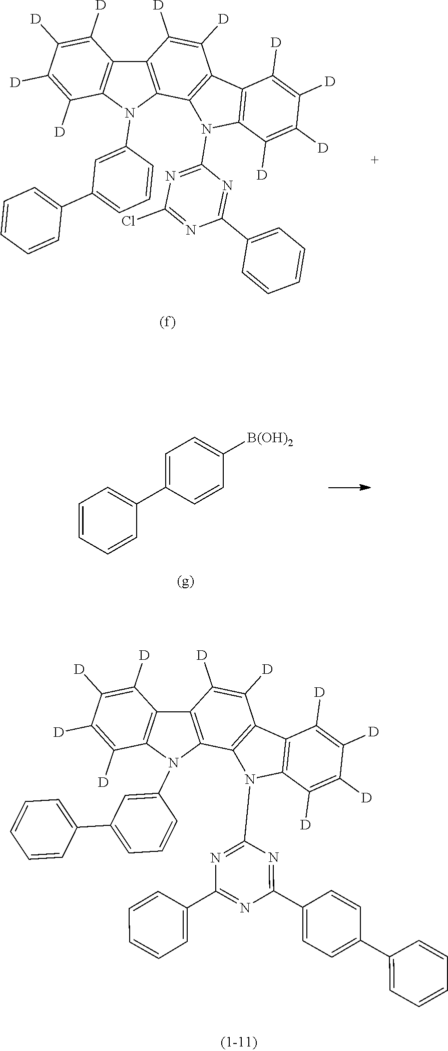 [SMILES: [2H]c1c([2H])c([2H])c2c(c1[2H])c1c([2H])c([2H])c3c4c([2H])c([2H])c([2H])c([2H])c4n(-c4nc(Cl)nc(-c5ccccc5)n4)c3c1n2-c1cccc(-c2ccccc2)c1 ; OB(O)c1ccc(-c2ccccc2)cc1 ; [2H]c1c([2H])c([2H])c2c(c1[2H])c1c([2H])c([2H])c3c4c([2H])c([2H])c([2H])c([2H])c4n(-c4nc(-c5ccccc5)nc(-c5ccc(-c6ccccc6)cc5)n4)c3c1n2-c1cccc(-c2ccccc2)c1]

To 3.50 g (0.00576 mol) of compound (f) were added 1.37 g (0.00691 mol) of compound (g), 0.001 g (0.00002 mol) of CX21 manufactured by Umicore, 1.59 g (0.0115 mol) of potassium carbonate, 21 g of m-xylene, and 3.5 g of distilled water, and the mixture was stirred under a nitrogen atmosphere at 90° C. for 3 hours. After the mixture was cooled to room temperature, 8.8 g of water was added, the mixture was cooled to room temperature and then stirred for 2 hours, and then the precipitated crystal was collected by filtration. The resulting solid was repeatedly purified by crystallization using 54 g of m-xylene to give 2.91 g (0.00400 mol, yield 66.1%, rate of deuteration 28%) of compound (1-11) as a yellow solid. (APCI-TOFMS, m/z 726 [M+H]⁺).

### Synthesis Example 5

### [C32]

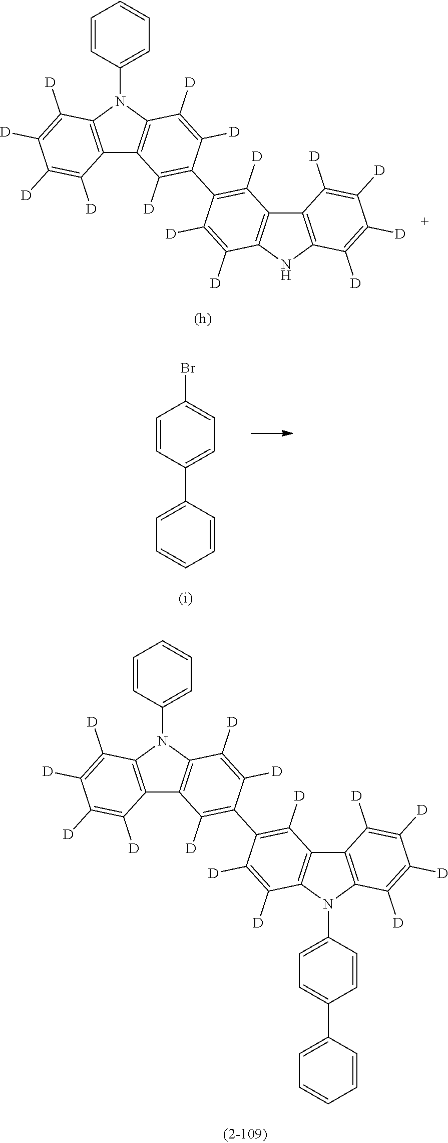 [SMILES: [2H]c1c([2H])c([2H])c2c([nH]c3c([2H])c([2H])c(-c4c([2H])c([2H])c5c(c4[2H])c4c([2H])c([2H])c([2H])c([2H])c4n5-c4ccccc4)c([2H])c32)c1[2H] ; Brc1ccc(-c2ccccc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccccc1]

To 3.0 g (7.10 mmol) of compound (h) were added 2.0 g (8.58 mmol) of compound (i), 100 mL of m-xylene, 0.2 g (0.39 mmol) of bis(tri-tert-butylphosphine)palladium, and 4.9 g (35.5 mmol) of potassium carbonate, and the mixture was stirred under a nitrogen atmosphere with heating and reflux for 5 hours. The reaction liquid was cooled, and then subjected to separation and purification to give 1.5 g (2.61 mmol, yield 37%, rate of deuteration 48%) of compound (2-109) as a white solid (APCI-TOFMS, m/z 575 [M+H]⁺).

### Synthesis Example 6

### [C33]

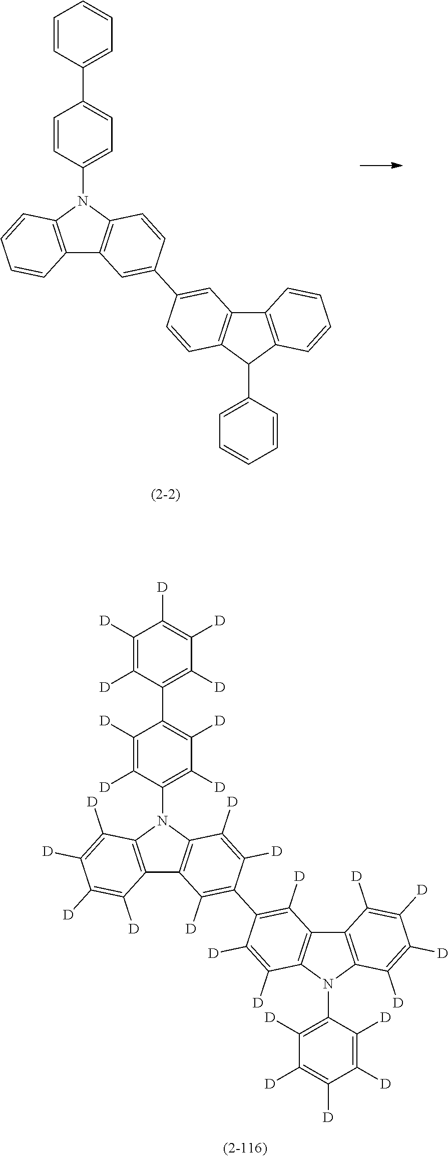 [SMILES: c1ccc(-c2ccc(-n3c4ccccc4c4cc(-c5ccc6c(c5)-c5ccccc5C6c5ccccc5)ccc43)cc2)cc1 ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]]

To 8.3 g (14.8 mmol) of compound (2-2) were added 160 mL of deuterated benzene (C₆D₆) and 10.0 g of deuterated trifluoromethanesulfonic acid (TfOD), and the mixture was heated and stirred under a nitrogen atmosphere at 50° C. for 6.5 hours. The reaction liquid was added to a solution of sodium carbonate (7.4 g) in deuterated water (200 mL), and the mixture was rapidly cooled, and subjected to separation and purification to give 2.5 g (4.25 mmol, yield 29%, rate of deuteration 81%) of compound (2-116) as a white solid (APCI-TOFMS, m/z 589 [M+H]⁺).

Compounds used in Examples and Comparative Examples are shown below.

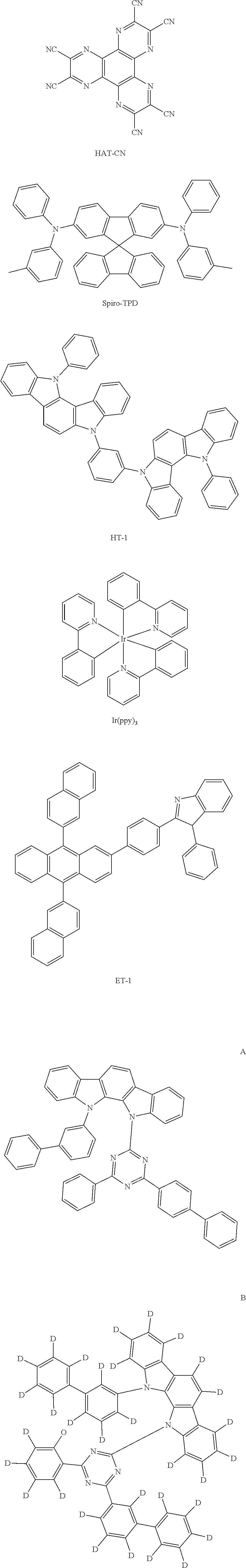

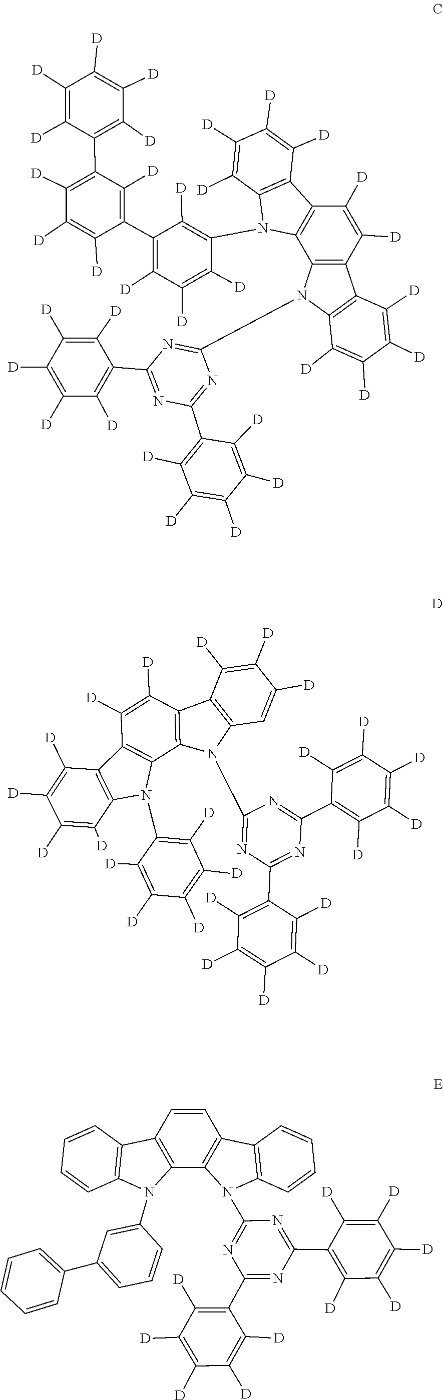

Reaction was performed in the same manner as in Synthesis Examples 1 to 6 to synthesize deuterated products 1-9, 1-10, 1-81, 1-85, 2-118, 2-119, and 2-120 being example compounds and deuterated products B, C, D, and E being comparative example compounds. Then, the rate of deuteration was calculated in the same manner as for compound 1-11, 2-109, and 2-116. Results are shown in Table 1.

TABLE 1

| | Rate of |
|---|---|
| Compound | deuteration |
| 1-9 | 27% |
| 1-10 | 27% |
| 1-81 | 24% |
| 1-85 | 23% |
| 2-118 | 81% |
| 2-119 | 80% |
| 2-120 | 79% |
| B | 92% |
| C | 91% |
| D | 91% |
| E | 31% |

### Example 1

On a glass substrate on which an anode made of ITO with a film thickness of 70 nm was formed, respective thin films were laminated by a vacuum evaporation method at a degree of vacuum of 4.0×10⁻⁵ Pa. First, HAT-CN was formed with a thickness of 25 nm as a hole injection layer on ITO, and next, Spiro-TPD was formed with a thickness of 30 nm as a hole transport layer. Next, HT-1 was formed with a thickness of 10 nm as an electron blocking layer. Then, as shown in Table 2, compound 1-1 as a host and Ir(ppy)₃ as a light-emitting dopant were co-vapor-deposited from different vapor deposition sources, respectively, to form a light-emitting layer with a thickness of 40 nm. In this case, co-vapor deposition was performed under vapor deposition conditions such that the concentration of Ir(ppy)₃ was 10 mass %. Next, ET-1 was formed with a thickness of 20 nm as an electron transport layer. Further, LiF was formed with a thickness of 1 nm as an electron injection layer on the electron transport layer. Finally, Al was formed with a thickness of 70 nm as a cathode on the electron injection layer to produce an organic EL device.

### Examples 2 to 5 and Comparative Examples 1 to 5

Organic EL devices were produced in the same manner as in Example 1 except that the compounds shown in Table 2 were used as the host in Example 1.

Evaluation results of the produced organic EL devices are shown in Table 2. In the table, the luminance, voltage, and power efficiency are values at a driving current of 10 mA/cm², and they exhibit initial characteristics. LT97 is a time period needed for the luminance to be reduced to 97% of the initial luminance that is assumed to be 100% at a driving current of 20 mA/cm², and it represents lifetime characteristics. The numbers with which the host compounds are marked are numbers with which the exemplified compounds are marked.

TABLE 2

| | | | | Power | |
|---|---|---|---|---|---|
| | Host | Voltage | Luminance | efficiency | LT97 |
| | compound | (V) | (cd/m²) | (lm/W) | (h) |
| Example 1 | 1-1 | 2.9 | 5500 | 59.6 | 90 |
| Example 2 | 1-10 | 3.0 | 6000 | 62.8 | 80 |
| Example 3 | 1-13 | 2.9 | 6000 | 65.0 | 80 |
| Example 4 | 1-14 | 3.0 | 5700 | 59.7 | 80 |
| Example 5 | 1-15 | 3.0 | 5700 | 59.7 | 70 |
| Comparative | A | 3.0 | 5700 | 59.7 | 70 |
| Example 1 | | | | | |
| Comparative | B | 3.1 | 5600 | 56.7 | 50 |
| Example 2 | | | | | |
| Comparative | C | 3.1 | 6400 | 64.8 | 60 |
| Example 3 | | | | | |
| Comparative | D | 3.1 | 5500 | 55.7 | 40 |
| Example 4 | | | | | |
| Comparative | E | 3.1 | 6600 | 66.9 | 40 |
| Example 5 | | | | | |

### Example 6

Each thin film was laminated on the glass substrate on which an anode made of ITO having a film thickness of 110 nm was formed by a vacuum deposition method at a degree of vacuum of 4.0×10⁻⁵ Pa. First, HAT-CN was formed with a thickness of 25 nm as a hole injection layer on ITO, and next, Spiro-TPD was formed with a thickness of 30 nm as a hole transport layer. Next, HT-1 was formed with a thickness of 10 nm as an electron blocking layer. Then, as shown in Table 3, compound 1-1 as a first host, compound 2-2 as a second host, and Ir(ppy)₃ as a light-emitting dopant were co-vapor-deposited from different vapor deposition sources, respectively, to form a light-emitting layer with a thickness of 40 nm. At this time, they were co-deposited under deposition conditions such that the concentration of Ir(ppy)₃ was 10 mass % and the weight ratio of the first host to the second host was 30:70. Next, ET-1 was formed with a thickness of 20 nm as an electron transport layer. Further, LiF was formed with a thickness of 1 nm as an electron injection layer on the electron transport layer. Finally, Al was formed with a thickness of 70 nm as a cathode on the electron injection layer to produce an organic EL device.

### Examples 7 to 27

Organic EL devices were produced in the same manner as in Example 6 except that compounds shown in Table 3 were used as the first host and the second host and the weight ratio was as shown in Table 3.

### Examples 28 to 39

Organic EL devices were produced in the same manner as in Example 6 except that a premixture obtained by weighing a first host and a second host shown in Table 3 at a weight ratio shown in Table 3 and mixing them while grinding in a mortar was vapor-deposited from one vapor deposition source.

### Comparative Examples 6 to 14

Organic EL devices were produced in the same manner as in Example 6 except that compounds shown in Table 4 were used as the first host and the second host and the weight ratio was as shown in Table 4.

### Comparative Examples 15 to 20

Organic EL devices were produced in the same manner as in Example 6 except that a premixture obtained by weighing a first host and a second host shown in Table 4 at a weight ratio shown in Table 4 and mixing them while grinding in a mortar was vapor-deposited from one vapor deposition source.

Evaluation results of the produced organic EL devices are shown in Table 3 and Table 4. In the tables, the luminance, voltage, and power efficiency are values at a driving current of 10 mA/cm², and they exhibit initial characteristics. LT97 is a time period needed for the luminance to be reduced to 97% of the initial luminance that is assumed to be 100% at a driving current of 20 mA/cm², and it represents lifetime characteristics. The weight ratio corresponds to first host:second host.

TABLE 3

| | | | | | | Power | |
|---|---|---|---|---|---|---|---|
| | First | Second | Weight | Voltage | Luminance | efficiency | LT97 |
| | host | host | ratio | (V) | (cd/m²) | (lm/W) | (h) |
| Example 6 | 1-1 | 2-2 | 30:70 | 3.8 | 7500 | 62.0 | 450 |
| Example 7 | 1-6 | 2-2 | 30:70 | 3.9 | 7500 | 60.4 | 430 |
| Example 8 | 1-9 | 2-2 | 30:70 | 3.9 | 7700 | 62.0 | 480 |
| Example 9 | 1-9 | 2-118 | 30:70 | 4.0 | 7900 | 62.1 | 480 |
| Example 10 | 1-9 | 2-120 | 30:70 | 4.0 | 7900 | 62.1 | 400 |
| Example 11 | 1-10 | 2-2 | 30:70 | 3.8 | 7500 | 62.0 | 360 |
| Example 12 | 1-10 | 2-116 | 30:70 | 4.0 | 7900 | 62.1 | 480 |
| Example 13 | 1-10 | 2-120 | 30:70 | 4.0 | 7900 | 62.1 | 390 |
| Example 14 | 1-11 | 2-2 | 30:70 | 3.8 | 7600 | 62.8 | 360 |
| Example 15 | 1-28 | 2-2 | 30:70 | 3.8 | 7500 | 62.0 | 400 |
| Example 16 | 1-11 | 2-40 | 30:70 | 3.8 | 7400 | 61.2 | 360 |
| Example 17 | 1-11 | 2-41 | 30:70 | 4.1 | 7400 | 56.7 | 390 |
| Example 18 | 1-11 | 2-42 | 30:70 | 4.1 | 7600 | 58.3 | 340 |
| Example 19 | 1-11 | 2-43 | 30:70 | 4.1 | 7300 | 56.0 | 380 |
| Example 20 | 1-11 | 2-109 | 30:70 | 4.0 | 7600 | 59.7 | 450 |
| Example 21 | 1-11 | 2-116 | 30:70 | 4.3 | 7600 | 55.5 | 540 |
| Example 22 | 1-11 | 2-118 | 30:70 | 4.3 | 7400 | 54.1 | 440 |
| Example 23 | 1-11 | 2-2 | 40:60 | 4.5 | 7400 | 51.7 | 360 |
| Example 24 | 1-11 | 2-2 | 50:50 | 3.8 | 7300 | 60.4 | 270 |
| Example 25 | 1-81 | 2-116 | 30:70 | 3.9 | 7700 | 62.0 | 450 |
| Example 26 | 1-85 | 2-116 | 30:70 | 3.9 | 7700 | 62.0 | 440 |
| Example 27 | 1-85 | 2-120 | 30:70 | 4.0 | 7800 | 61.3 | 400 |
| Example 28 | 1-11 | 2-2 | 30:70 | 3.7 | 7900 | 67.1 | 360 |
| Example 29 | 1-11 | 2-109 | 30:70 | 3.6 | 7800 | 68.1 | 460 |
| Example 30 | 1-11 | 2-116 | 30:70 | 4.1 | 7800 | 59.8 | 550 |
| Example 31 | 1-11 | 2-109 | 50:50 | 4.0 | 7000 | 55.0 | 330 |
| Example 32 | 1-11 | 2-116 | 50:50 | 4.5 | 7200 | 50.3 | 360 |
| Example 33 | 1-9 | 2-118 | 30:70 | 4.0 | 8200 | 65.6 | 510 |
| Example 34 | 1-9 | 2-120 | 30:70 | 4.0 | 8100 | 64.9 | 420 |
| Example 35 | 1-10 | 2-116 | 30:70 | 4.0 | 8200 | 65.6 | 500 |
| Example 36 | 1-10 | 2-120 | 30:70 | 4.0 | 8100 | 64.9 | 420 |
| Example 37 | 1-81 | 2-116 | 30:70 | 3.9 | 8000 | 65.6 | 490 |
| Example 38 | 1-85 | 2-116 | 30:70 | 3.9 | 8000 | 65.6 | 480 |
| Example 39 | 1-85 | 2-120 | 30:70 | 4.0 | 8000 | 64.1 | 420 |

TABLE 4

| | | | | | | Power | |
|---|---|---|---|---|---|---|---|
| | First | Second | Weight | Voltage | Luminance | efficiency | LT97 |
| | host | host | ratio | (V) | (cd/m²) | (lm/W) | (h) |
| Comparative Example 6 | A | 2-2 | 30:70 | 3.8 | 7500 | 61.5 | 300 |
| Comparative Example 7 | B | 2-2 | 30:70 | 4.4 | 7900 | 57.0 | 310 |
| Comparative Example 8 | C | 2-2 | 30:70 | 4.3 | 8000 | 58.7 | 250 |
| Comparative Example 9 | D | 2-2 | 30:70 | 4.4 | 8000 | 57.7 | 290 |
| Comparative Example 10 | E | 2-2 | 30:70 | 4.5 | 7800 | 54.4 | 220 |
| Comparative Example 11 | B | 2-40 | 30:70 | 3.5 | 7700 | 69.3 | 250 |
| Comparative Example 12 | B | 2-41 | 30:70 | 3.5 | 8200 | 73.1 | 250 |
| Comparative Example 13 | B | 2-42 | 30:70 | 3.5 | 7300 | 65.5 | 220 |
| Comparative Example 14 | B | 2-43 | 30:70 | 3.6 | 7300 | 63.7 | 250 |
| Comparative Example 15 | A | 2-2 | 30:70 | 3.7 | 7400 | 62.8 | 290 |
| Comparative Example 16 | A | 2-2 | 50:50 | 3.3 | 7000 | 67.4 | 200 |
| Comparative Example 17 | B | 2-2 | 30:70 | 3.7 | 7900 | 67.0 | 280 |
| Comparative Example 18 | B | 2-2 | 50:50 | 3.3 | 7500 | 72.2 | 190 |
| Comparative Example 19 | C | 2-2 | 30:70 | 3.7 | 8000 | 67.9 | 250 |
| Comparative Example 20 | C | 2-2 | 50:50 | 3.3 | 7800 | 75.1 | 170 |

From the results in Tables 2 to 4, it is understood that the organic EL devices according to Examples exhibited good characteristics as compared with the organic EL devices according to Comparative Examples. That is, it is understood that Examples 1 to 5 in which the host material of the general formula (1) in the present invention was used improved the lifetime as compared with Comparative Examples 1 to 5 in which any of other host materials was used. In addition, it is understood that Examples 6 to 39 in which the compound of the general formula (1) in the present invention was used as the first host material and the compound of the general formula (6) in the present invention was used as the second host material also improved the lifetime as compared with Comparative Examples 6 to 20 not using such a combination.

Among these, when a mixed host of a first host material and a second host material is used, it is understood that Example 28 to 39 obtained by premixing these host materials and vapor-depositing the premixture from one vapor deposition source can give devices having high efficiency and extended lifetime with good balance in spite of having a low voltage, as compared with Examples 7 to 27 obtained by co-vapor-depositing these host materials from different vapor deposition sources without being premixed.

Table 5 shows the temperature at 50% weight loss (T₅₀) of compounds 1-9, 1-10, 1-11, 1-81, 1-85, 2-2, 2-109, 2-116, 2-118, 2-119, and 2-120, and compounds A, B, and C.

TABLE 5

| Compound | T₅₀[° C.] |
|---|---|
| 1-9 | 286 |
| 1-10 | 276 |
| 1-11 | 263 |
| 1-81 | 262 |
| 1-85 | 272 |
| 2-2 | 265 |
| 2-109 | 267 |
| 2-116 | 266 |
| 2-118 | 297 |
| 2-119 | 308 |
| 2-120 | 291 |
| A | 286 |
| B | 259 |
| C | 273 |

### REFERENCE SIGNS LIST

1: substrate, 2: anode, 3: hole injection layer, 4: hole transport layer, 5: light-emitting layer, 6: electron transport layer, 7: cathode

1. A host material for an organic electroluminescent device represented by the following general formula (1): 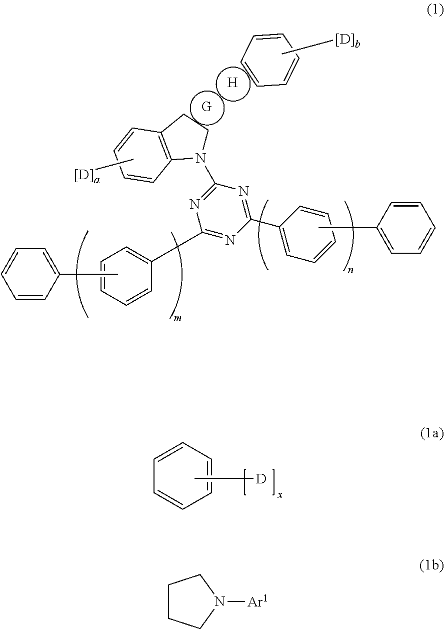
wherein, ring G represents an aromatic ring represented by a formula (1a), ring H represents a heterocyclic ring represented by a formula (1b),
D represents deuterium, and Ar¹ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five aromatic rings of these aromatic groups are linked to each other,
a, b, and x represent the number of substitutions, a and b each independently represent an integer of 0 to 4, x represents an integer of 0 to 2, and a+b+x≥1, and m and n represent the number of repetitions, m and n each independently represent an integer of 0 or 1, and m+n is 1 or more.

2. The host material according to claim 1, wherein Ar¹ represents a substituted or unsubstituted aromatic hydrocarbon group having 6 to 18 carbon atoms or a substituted or unsubstituted linked aromatic group in which two aromatic rings of these aromatic groups are linked to each other in the general formula (1).

3. The host material according to claim 1, wherein Ar¹ is a substituted or unsubstituted phenyl group or a substituted or unsubstituted biphenyl group in the general formula (1).

4. The host material according to claim 1, wherein m represents an integer of 0 in the general formula (1).

5. The host material according to claim 1, wherein the compound represented by the general formula (1) is represented by any of the following formulas (2) to (5): 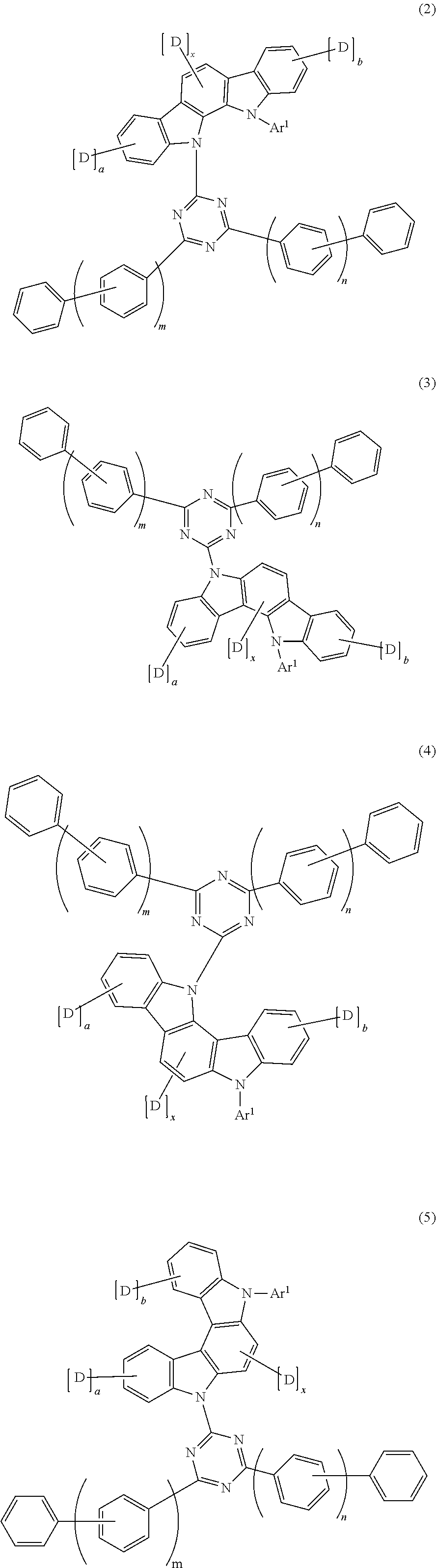
wherein Ar¹, a, b, x, m, and n have the same meaning as in the general formula (1).

6. The host material according to claim 5, wherein the compound represented by the general formula (1) is represented by the formula (2).

7. The host material according to claim 1, wherein a+b+x=10 in the general formula (1).

8. An organic electroluminescent device comprising one or more light emitting layers between an anode and a cathode opposed to each other, wherein at least one of the light emitting layers contains a first host material selected from the host materials according to claim 1, a second host material selected from a compound represented by the following general formula (6), and a light emitting dopant material: 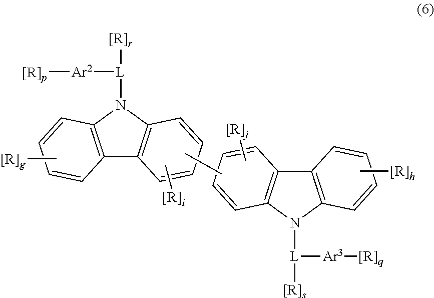
wherein, Ar² and Ar³ each independently represent a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five aromatic rings of these aromatic groups are linked to each other, each L independently represents a single bond, a substituted or unsubstituted aromatic hydrocarbon group having 6 to 18 carbon atoms, or a substituted or unsubstituted aromatic heterocyclic group having 3 to 17 carbon atoms, each R independently represents deuterium or an aliphatic hydrocarbon group having 1 to 10 carbon atoms,
g to j and p to s represent the number of substitutions, g, h, r, and s each independently represent an integer of 0 to 4, i and j each independently represent an integer of 0 to 3, and p and q each independently represent an integer of 0 to 13, provided that when L is a single bond, r and s represent an integer of 0.

9. The organic electroluminescent device according to claim 8, wherein the general formula (6) is represented by the following formula (7): 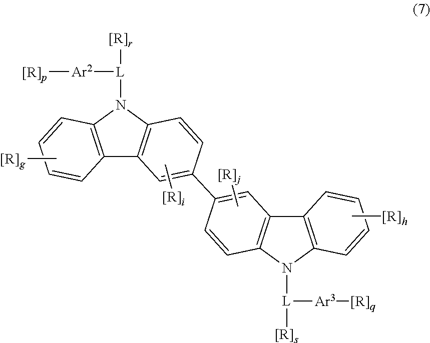
wherein Ar², Ar³, L, R, g to j, and p to s are as defined for the general formula (6).

10. The organic electroluminescent device according to claim 8, wherein Ar² and Ar³ each independently represent a substituted or unsubstituted phenyl group, a substituted or unsubstituted biphenyl group, or a substituted or unsubstituted terphenyl group in the general formula (6).

11. The organic electroluminescent device according to claim 8, wherein R is deuterium in the general formula (6).

12. The organic electroluminescent device according to claim 8, wherein g+h+i+j=14 in the general formula (6).

13. The organic electroluminescent device according to claim 8, wherein the light emitting dopant material is an organic metal complex containing at least one metal selected from ruthenium, rhodium, palladium, silver, rhenium, osmium, iridium, platinum and gold.

14. The organic electroluminescent device according to claim 8, wherein the light emitting dopant material is a thermally activated delayed fluorescence-emitting dopant material.

15. A premixture used for forming a light-emitting layer for an organic electroluminescent device comprising a light-emitting layer containing hosts and a light-emitting dopant material between an anode and a cathode opposed to each other, wherein the premixture contains a first host and a second host, the first host is selected from a compound represented by the following general formula (1), and the second host is selected from a compound represented by the following general formula (6): 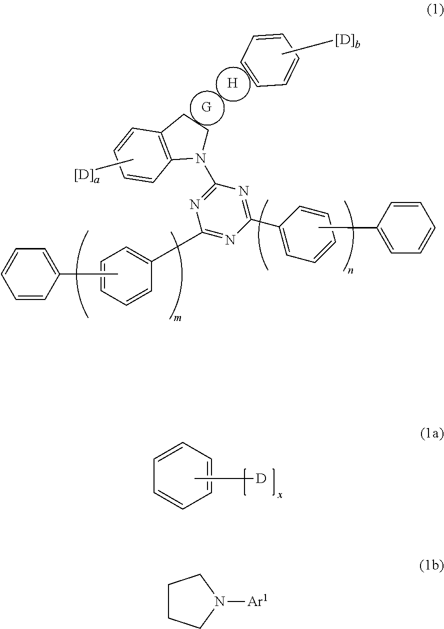
wherein, ring G represents an aromatic ring represented by a formula (1a), ring H represents a heterocyclic ring represented by a formula (1b),
D represents deuterium, and Ar¹ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five aromatic rings of these aromatic groups are linked to each other,
a, b, and x represent the number of substitutions, a and b each independently represent an integer of 0 to 4, x represents an integer of 0 to 2, and a+b+x≥1, and m and n represent the number of repetitions, m and n each independently represent an integer of 0 or 1, and m+n is 1 or more, 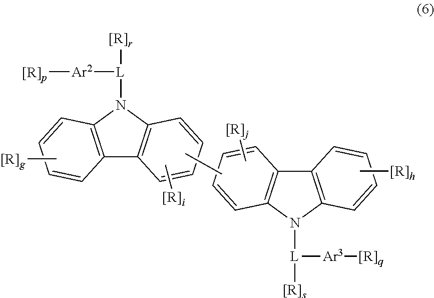
wherein, Ar² and Ar³ each independently represent a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five of these aromatic groups are linked to each other,
each L independently represents a single bond, a substituted or unsubstituted aromatic hydrocarbon group having 6 to 18 carbon atoms, or a substituted or unsubstituted aromatic heterocyclic group having 3 to 17 carbon atoms, each R independently represents deuterium or an aliphatic hydrocarbon group having 1 to 10 carbon atoms,
g to j and p to s represent the number of substitutions, g, h, r, and s each independently represent an integer of 0 to 4, i and j each independently represent an integer of 0 to 3, and p and q each independently represent an integer of 0 to 13, provided that when L is a single bond, r and s represent an integer of 0.

16. The premixture according to claim 15, wherein a difference in temperature at 50% weight loss of the first host and the second host is within 20° C.

17. A method for producing the organic electroluminescent device according to claim 8, comprising a step of mixing the first host and the second host to give a premixture and then vapor-depositing a host material containing the premixture to form a light emitting layer.
